(19) United States
(12) Patent Application Publication
(10) Pub. No.: US 2006/0217300 A1
(43) Pub. Date: Sep. 28, 2006

(54) Analogues of glp-1

(75) Inventor: Zheng Xin Dong, HOLLISTON, MA (US)

Correspondence Address: Brian R Morrill;Biomeasure Incorporated, 27 Maple Street, Milford, MA, 01757-3650, US

(73) Assignee: BIOMEASURE, INCORPORATED, MILFORD, MA (US)

(21) Appl. No.: 10/546,303

(22) PCT Filed: Feb. 17, 2004
(86) PCT No.: PCT/US04/04421

§ 371 Date: Aug. 19, 2005

Related U.S. Application Data
(60) Provisional application No. 60/449,203, filed on Feb. 19, 2003.

(51) Int. Cl. A61K 38/26

(52) U.S. Cl. 514/12; 530/324

(57) ABSTRACT

The present invention is directed to peptide analogues of glucagon-like peptide-1, the pharmaceutically-acceptable salts thereof, to methods of using such analogues to treat mammals, and to pharmaceutical compositions useful therefor comprising said analogues.

### SEQUENCE LISTING

The instant application contains a “lengthy” Sequence Listing which has been submitted via four CD-R in lieu of a printed paper copy, and is hereby incorporated by reference in its entirety. Said CD-R, recorded on Feb. 10, 2004, are labeled “CRF”, “Copy 1”, “Copy 2” and “Copy 3” respectively, and each contains only one identical 456 Kb file (129PPCT2.APP).

### BACKGROUND OF THE INVENTION

The present invention is directed to peptide analogues of glucagon-like peptide-1, the pharmaceutically-acceptable salts thereof, to methods of using such analogues to treat mammals and to pharmaceutical compositions useful therefore comprising said analogues.

Glucagon-like peptide-1 (7-36) amide (GLP-1)(SEQ ID NO: 775) is synthesized in the intestinal L-cells by tissue-specific post-translational processing of the glucagon precursor preproglucagon (Vamdell, J. M., et al., J. Histochem Cytochem, 1985:33:1080-6) and is released into the circulation in response to a meal. The plasma concentration of GLP-1 rises from a fasting level of approximately 15 pmol/L to a peak postprandial level of 40 pmol/L. It has been demonstrated that, for a given rise in plasma glucose concentration, the increase in plasma insulin is approximately threefold greater when glucose is administered orally compared with intravenously (Kreymann, B., et al., Lancet 1987:2, 1300-4). This alimentary enhancement of insulin release, known as the incretin effect, is primarily humoral and GLP-1 is now thought to be the most potent physiological incretin in humans. In addition to the insulinotropic effect, GLP-1 suppresses glucagon secretion, delays gastric emptying (Wettergren A., et al., Dig Dis Sci 1993:38:665-73) and may enhance peripheral glucose disposal (D'Alessio, D. A. et al., J. Clin Invest 1994:93:2293-6).

In 1994, the therapeutic potential of GLP-1 was suggested following the observation that a single subcutaneous (s/c) dose of GLP-1 could completely normalize postprandial glucose levels in patients with non-insulin-dependent diabetes mellitus (NIDDM) (Gutniak, M. K., et al., Diabetes Care 1994:17:1039-44). This effect was thought to be mediated both by increased insulin release and by a reduction in glucagon secretion. Furthermore, an intravenous infusion of GLP-1 has been shown to delay postprandial gastric emptying in patients with NIDDM (Williams, B., et al., J. Clin Endo Metab 1996:81:327-32). Unlike sulphonylureas, the insulinotropic action of GLP-1 is dependent on plasma glucose concentration (Holz, G. G. 4^{th}, et al., Nature 1993:361:362-5). Thus, the loss of GLP-1-mediated insulin release at low plasma glucose concentration protects against severe hypoglycemia. This combination of actions gives GLP-1 unique potential therapeutic advantages over other agents currently used to treat NIDDM.

Numerous studies have shown that when given to healthy subjects, GLP-1 potently influences glycemic levels as well as insulin and glucagon concentrations (Orskov, C, Diabetologia 35:701-711, 1992; Holst, J. J., et al., Potential of GLP-1 in diabetes management in Glucagon III, Handbook of Experimental Pharmacology, Lefevbre P J, Ed. Berlin, Springer Verlag, 1996, p. 311-326), effects which are glucose dependent (Kreymann, B., et al., Lancet ii: 1300-1304, 1987; Weir, G. C., et al., Diabetes 38:338-342, 1989). Moreover, it is also effective in patients with diabetes (Gutniak, M., N. Engl J Med 226:1316-1322, 1992; Nathan, D. M., et al., Diabetes Care 15:270-276, 1992), normalizing blood glucose levels in type 2 diabetic subjects (Nauck, M. A., et al., Diagbetologia 36:741-744, 1993), and improving glycemic control in type 1 patients (Creutzfeldt, W. O., et al., Diabetes Care 19:580-586, 1996), raising the possibility of its use as a therapeutic agent.

GLP-1 is, however, metabolically unstable, having a plasma half-life (t_{1/2}) of only 1-2 min in vivo. Exogenously administered GLP-1 is also rapidly degraded (Deacon, C. F., et al., Diabetes 44:1126-1131, 1995). This metabolic instability limits the therapeutic potential of native GLP-1. Hence, there is a need for GLP-1 analogues that are more active or are more metabolically stable than native GLP-1.

### SUMMARY OF THE INVENTION

In one aspect, the present invention is directed to a compound of formula (I),
(R²R³)-A⁷⁻A⁸-A⁹-A¹⁰-A¹¹-A¹²-A¹³-A¹⁴-A¹⁵-A¹⁶-A¹⁷⁻A¹⁸-A¹⁹-A²⁰-A²¹-A²²-A²³-A²⁴-A²⁵-A²⁶-A²⁷⁻A²⁸-A²⁹-A³⁰-A³¹-A³²-A³³-A³⁵-A³⁶-A³⁷⁻A³⁸-A³⁹-R¹, (I)
wherein:
A⁷ is L-His, Ura, Paa, Pta, Amp, Tma-His, des-amino-His, or deleted;
A⁸ is Abu, β-Ala, Ser, Gly or Val;
A⁹ is Glu, N-Me-Glu, N-Me-Asp or Asp;
A¹⁰ is Gly, Acc, β-Ala or Aib;
A¹¹ is Thr or Ser;
A¹² is Phe, Acc, Aic, Aib, 3-Pal, 4-Pal, 1-Nal, 2-Nal, Cha, Trp or X¹-Phe;
A¹³ is Thr or Ser;
A¹⁴ is Ser or Aib;
A¹⁵ is Asp or Glu;
A¹⁶ is Val, Acc, Aib, Leu, Ile, Tle, Nle, Abu, Ala or Cha;
A¹⁷ is Aib, Ser or Thr;
A¹⁸ is Lys, Ser or Thr;
A¹⁹ is Tyr, Cha, Phe, 3-Pal, 4-Pal, Acc, 1-Nal, 2-Nal, or X¹-Phe;
A²⁰ is Leu, Acc, Aib, Nle, Ile, Cha, Tle, Val, Phe or X¹-Phe;
A²¹ is Glu or Asp;
A²² is Gly, Acc, β-Ala, Glu or Aib;
A²³ is Gln, Asp, Asn or Glu;
A²⁴ is Ala, Aib, Val, Abu, Tle or Acc;
A²⁵ is Ala, Aib, Val, Abu, Tle, Acc, Lys, Arg, hArg, Orn, HN—CH((CH₂)ₙ—N(R¹⁰R¹¹))—C(O) or HN—CH((CH₂)ₑ—X³)—C(O);
A²⁶ is Lys, Arg, hArg, Orn, HN—CH((CH₂)ₙ—N(R¹⁰R¹¹))—C(O) or HN—CH((CH₂)ₑ—X³)—C(O);
A²⁷ is Glu, Asp, Leu, Aib or Lys;
A²⁸ is Phe, 3-Pal, 4-Pal, 1-Nal, 2-Nal, X¹-Phe, Aic, Acc, Aib, Cha or Trp;
A²⁹ is Ile, Acc, Aib, Leu, Nle, Cha, Tle, Val, Abu, Ala or Phe;
A³⁰ is Ala, Aib or Acc;
A³¹ is Trp,-1-Nal, 2-Nal, 3-Pal, 4-Pal, Phe, Acc, Aib or Cha;
A³² is Leu, Acc, Aib, Nle, Ile, Cha, Tle, Phe, X¹-Phe or Ala;
A³³ is Val, Acc, Aib, Leu, Ile, Tle, Nle, Cha, Ala, Phe, Abu, Lys or X¹-Phe;
A³⁴ is Lys, Arg, hArg, Orn, HN—CH((CH₂)ₙ—N(R¹⁰R¹¹))—C(O) or HN—CH((CH₂)ₑ—X³)—C(O);
A³⁵ is Gly, β-Ala, D-Ala, Gaba, Ava, HN—(CH₂)ₘ—C(O), Aib, Acc or a D-amino acid;
A³⁶ is L- or D-Arg, D- or L-Lys, D- or L-hArg, D- or L-Orn, HN—CH((CH₂)ₙ—N(R¹⁰R¹¹))—C(O), HN—CH((CH₂)ₑ—X³)—C(O) or deleted;
A³⁷ is Gly, β-Ala, Gaba, Ava, Aib, Acc, Ado, Arg, Asp, Aun, Aec, HN—(CH₂)ₘ—C(O), HN—CH((CH₂)ₙ—N(R¹⁰R¹¹))—C(O), HN—CH((CH₂)ₙ—O(R¹⁰))—C(O), a D-amino acid, or deleted;
A³⁸ is D- or L-Lys, D- or L-Arg, D- or L-hArg, D- or L-Orn, HN—CH((CH₂)ₙ—N(R¹⁰R¹¹))—C(O), HN—CH((CH₂)ₑ—X³)—C(O) Ava, Ado, Aec or deleted;
A³⁹ is D- or L-Lys, D- or L-Arg, HN—CH((CH₂)ₙ—N(R¹⁰R¹¹))—C(O), Ava, Ado, Aec, or Aun, or is deleted;
X¹ is, independently for each occurrence, selected from the group consisting of (C₁-C₆)alkyl, OH and halo;
R¹ is OH, NH₂, (C₁-C₃₀)alkoxy, or NH—X²—CH₂-Z⁰, wherein X² is a (C₁-C₁₂)hydrocarbon moiety, and Z⁰ is H, OH, CO₂H or CONH₂; 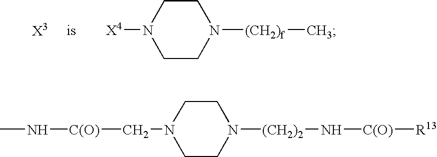
or —C(O)—NHR¹², wherein X⁴ is, independently for each occurrence, —C(O)—, —NH—C(O)— or —CH₂—, and wherein f is, independently for each occurrence, an integer from 1 to 29 inclusive;
each of R² and R³ is independently selected from the group consisting of H, (C₁-C₃₀)alkyl, (C₂-C₃₀)alkenyl, phenyl(C₁-C₃₀)alkyl, naphthyl(C₁-C₃₀)alkyl, hydroxy(C₁-C₃₀)alkyl, hydroxy(C₂-C₃₀)alkenyl, hydroxyphenyl(C₁-C₃₀)alkyl, and hydroxynaphthyl(C₁-C₃₀)alkyl; or one of R² and 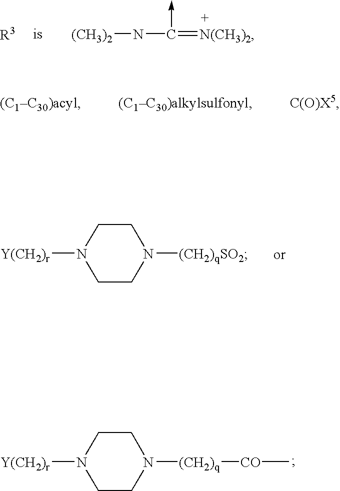
   wherein Y is H, OH or NH₂; r is 0 to 4; q is 0 to 4; and X⁵ is (C₁-C₃₀)alkyl, (C₂-C₃₀)alkenyl, phenyl(C₁-C₃₀)alkyl, naphthyl(C₁-C₃₀)alkyl; hydroxy(C₁-C₃₀)alkyl, hydroxy(C₂-C₃₀)alkenyl, hydroxyphenyl(C₁-C₃₀)alkyl or hydroxynaphthyl(C₁-C₃₀)alkyl;
e is, independently for each occurrence, an integer from 1 to 4 inclusive;
m is, independently for each occurrence, an integer from 5 to 24 inclusive;
n is, independently for each occurrence, an integer from 1 to 5, inclusive;
each of R¹⁰ and R¹¹ is, independently for each occurrence, H, (C₁-C₃₀)alkyl, (C₁-C₃₀)acyl, (C₁-C₃₀)alkylsulfonyl, —C((NH)(NH₂)) or 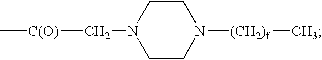
   and
R¹² and R¹³ each is, independently for each occurrence, (C₁-C₃₀)alkyl;
provided that:
(i) said compound is not:
(Ser⁸, β-Ala³⁵)hGLP-1(7-36)NH₂(SEQ ID NO.779);
(Gly⁸, Aib³⁵)hGLP-1(7-36)NH₂(SEQ ID NO.780); or
(Gly⁸, β-Ala³⁵)hGLP-1(7-36)NH₂(SEQ ID NO.781);
(ii) when A⁷ is Ura, Paa or Pta, then R² and R³ are deleted; and
(iii) when R¹⁰ is (C₁-C₃₀)acyl, (C₁-C₃₀)alkylsulfonyl, —C((NH)(NH₂)) or 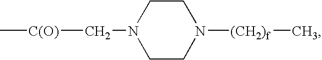
   then R¹¹ is H or (C₁-C₃₀)alkyl;
or a pharmaceutically acceptable salt thereof.

A preferred group of compounds of formula (I) is where A¹¹ is Thr; A¹³ is Thr; A¹⁵ is Asp; A¹⁷ is Ser; A¹⁸ is Ser; A²¹ is Glu; A²³ is Gln or Glu; A²⁷ is Glu; and A³¹ is Trp; or a pharmaceutically acceptable salt thereof.

A preferred group of compounds of the immediately foregoing group of compounds is where A⁹ is Glu, N-Me-Glu or N-Me-Asp; A¹² is Phe, Acc or Aic; A¹⁶ is Val, Acc or Aib; A¹⁹ is Tyr; A²⁰ is Leu, Acc or Cha; A²⁴ is Ala, Aib or Acc; A²⁵ is Ala, Aib, Acc, Lys, Arg, hArg, Orn, HN—CH((CH₂)ₙ—N(R¹⁰R¹¹))—C(O) or HN—CH((CH₂)ₑ—X³)—C(O); A²⁸ is Phe; A²⁹ is Ile or Acc; A³⁰ is Ala or Aib; A³² is Leu, Acc or Cha; and A³³ is Val or Acc; or a pharmaceutically acceptable salt thereof.

A preferred group of compounds of the immediately foregoing group of compounds is where A¹⁰ is Gly; A¹² is Phe, A6c or A5c; A¹⁶ is Val, A6c or A5c; A²⁰ is Leu, A6c, A5c or Cha; A²² is Gly, β-Ala or Aib; A²⁴ is Ala or Aib; A²⁹ is Ile, A6c or A5c; A³² is Leu, A6c, A5c or Cha; A³³ is Val, A6c or A5c; A³⁵ is Aib, β-Ala, Ado, A6c, A5c or Gly; and A³⁷ is Gly, Aib, β-Ala, Ado, D-Ala or deleted; or a pharmaceutically acceptable salt thereof.

A preferred group of compounds of the immediately foregoing group of compounds is where X⁴ for each occurrence is —C(O)—; e is, independently for each occurrence, 1 or 2; and R¹ is OH or NH₂; or a pharmaceutically acceptable salt thereof.

A preferred group of compounds according to the immediately foregoing group of compounds is where R² is H and R³ is (C₁-C₃₀)alkyl, (C₂-C30)alkenyl, (C₁-C₃₀)acyl, (C₁-C₃₀alkysulfonyl, 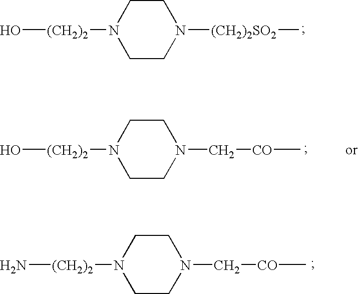
or a pharmaceutically acceptable salt thereof.

Another preferred group of compounds from among the compounds according to paragraph [063] is where R¹⁰ is (C₁-C₃₀)acyl, (C₁-C₃₀)alkylsulfonyl or 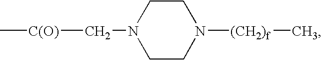
and R¹¹ is H; or a pharmaceutically acceptable salt thereof.

A preferred group of compounds of the immediately foregoing group of compounds is where R¹⁰ is (C₄-C₂₀)acyl, (C₄-C₂₀)alkylsulfonyl or 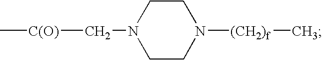
or a pharmaceutically acceptable salt thereof

Another preferred group of compounds from among the compounds according to paragraph [063], is where A⁸ is Gly, Ser, or Val; or a pharmaceutically acceptable salt thereof.

A preferred group of compounds of the immediately foregoing group of compounds is where A⁸ is Gly or Ser; or a pharmaceutically acceptable salt thereof.

A preferred compound of the immediately foregoing group of compounds is a compound according to the formula:

| | |
|---|---|
| ((N^{α}-Me-His)⁷,Gly⁸,Aib³⁵)hGLP-1(7-36)NH₂; | (SEQ ID NO.1) |
| ((N^{α}-Me-His)⁷,Gly⁸,β-Ala³⁵)hGLP-1(7-36)NH₂; | (SEQ ID NO.2) |
| ((N^{α}-Me-His)⁷,Gly⁸,Aib³⁵,Arg^{26,34})hGLP-1(7-36)NH₂; | (SEQ ID NO.3) |
| ((N^{α}-Me-His)⁷,Gly⁸,Arg^{26,34},β-Ala³⁵)hGLP-1(7-36)NH₂; | (SEQ ID NO.4) |
| (Gly⁸,A6c³⁵)hGLP-1(7-36)NH₂; | (SEQ ID NO.5) |
| (Gly⁸,A5c³⁵)hGLP-1(7-36)NH₂; | (SEQ ID NO.6) |
| (Gly⁸,D-Ala³⁵)hGLP-1(7-36)NH₂; | (SEQ ID NO.7) |
| (Gly⁸,Aib³⁵,A6c³²)hGLP-1(7-36)NH₂; | (SEQ ID NO.8) |
| (Gly⁸,Aib³⁵,A5c³²)hGLP-1(7-36)NH₂; | (SEQ ID NO.9) |
| (Gly⁸,Aib³⁵,Glu²³)hGLP-1(7-36)NH₂; | (SEQ ID NO.10) |
| (Gly⁸,Aib^{24,35})hGLP-1(7-36)NH₂; | (SEQ ID NO.11) |
| (Gly⁸,Aib^{30,35})hGLP-1(7-36)NH₂; | (SEQ ID NO.12) |
| (Gly⁸,Aib^{25,35})hGLP-1(7-36)NH₂; | (SEQ ID NO.13) |
| (Gly⁸,Aib³⁵,A6c^{16,20})hGLP-1(7-36)NH₂; | (SEQ ID NO.14) |
| (Gly⁸,Aib³⁵,A6c^{16,29,32})hGLP-1(7-36)NH₂; | (SEQ ID NO.15) |
| (Gly⁸,Aib³⁵,A6c^{20,32})hGLP-1(7-36)NH₂; | (SEQ ID NO.16) |
| (Gly⁸,Aib³⁵,A6c²⁰)hGLP-1(7-36)NH₂; | (SEQ ID NO.17) |
| (Gly⁸,Aib³⁵,Lys²⁵)hGLP-1(7-36)NH₂; | (SEQ ID NO.18) |
| (Gly⁸,Aib^{24,35},A6c²⁰)hGLP-1(7-36)NH₂; | (SEQ ID NO.19) |
| (Gly⁸,Aib³⁵,A6c^{29,32})hGLP-1(7-36)NH₂; | (SEQ ID NO.20) |
| (Gly⁸,Aib^{24,35},A6c^{29,32})hGLP-1(7-36)NH₂: | (SEQ ID NO.21) |
| (Gly⁸,Aib³⁵,A6c¹²)hGLP-1(7-36)NH₂; | (SEQ ID NO.22) |
| (Gly⁸,Aib³⁵,Cha²⁰)hGLP-1(7-36)NH₂; | (SEQ ID NO.23) |
| (Gly⁸,Aib³⁵,A6c³³)hGLP-1(7-36)NH₂; | (SEQ ID NO.24) |
| (Gly⁸,A6c^{16,20},β-Ala³⁵)hGLP-1(7-36)NH₂; | (SEQ ID NO.25) |
| (Gly⁸,Aib³⁵,β-Ala²²)hGLP-1(7-36)NH₂; | (SEQ ID NO.26) |
| (Gly⁸,Aib^{22,35})hGLP-1(7-36)NH₂; | (SEQ ID NO.27) |
| (Gly⁸,Aib³⁵,Glu²³,A6c³²)hGLP-1(7-36)NH₂; | (SEQ ID NO.28) |
| (Gly⁸,Aib^{24,35},Glu²³,A6c³²)hGLP-1(7-36)NH₂; | (SEQ ID NO.29) |
| (Gly⁸,Aib^{24,25,35},Glu²³,A6c³²)hGLP-1(7-36)NH₂; | (SEQ ID NO.30) |
| (Gly⁸,Aib^{24,25,35},A6c^{16,20,32},Glu²³)hGLP-1(7-36)NH₂; | (SEQ ID NO.31) |
| (Gly⁸,A6c³²,β-Ala³⁵)hGLP-1(7-36)NH₂; | (SEQ ID NO.32) |
| (Gly⁸,A5C32,β-Ala³⁵)hGLP-1(7-36)NH₂; | (SEQ ID NO.33) |
| (Gly⁸,Glu²³,β-Ala³⁵)hGLP-1(7-36)NH₂; | (SEQ ID NO.34) |
| (Gly⁸,Aib²⁴,β-Ala³⁵)hGLP-1(7-36)NH₂; | (SEQ ID NO.35) |
| (Gly⁸,Aib³⁰,β-Ala³⁵)hGLP-1(7-36)NH₂; | (SEQ ID NO.36) |
| (Gly⁸,Aib²⁵,β-Ala³⁵)hGLP-1(7-36)NH₂; | (SEQ ID NO.37) |
| (Gly⁸,A6c^{16,29,32},β-Ala³⁵)hGLP-1(7-36)NH₂; | (SEQ ID NO.38) |
| (Gly⁸,A6c^{20,32},β-Ala³⁵)hGLP-1(7-36)NH₂; | (SEQ ID NO.39) |
| (Gly⁸,A6c²⁰,β-Ala³⁵)hGLP-1(7-36)NH₂; | (SEQ ID NO.40) |
| (Gly⁸,Lys²⁵,β-Ala³⁵)hGLP-1(7-36)NH₂; | (SEQ ID NO.41) |
| (Gly⁸,Aib²⁴,A6c²⁰,β-Ala³⁵)hGLP-1(7-36)NH₂; | (SEQ ID NO.42) |
| (Gly⁸,A6c^{29,32},β-Ala³⁵)hGLP-1(7-36)NH₂; | (SEQ ID NO.43) |
| (Gly⁸,Aib²⁴,A6c^{29,32},β-Ala³⁵)hGLP-1(7-36)NH₂; | (SEQ ID NO.44) |
| (Gly⁸,A6c¹²,β-Ala³⁵)hGLP-1(7-36)NH₂; | (SEQ ID NO.45) |
| (Gly⁸,Cha²⁰,β-Ala³⁵)hGLP-1(7-36)NH₂; | (SEQ ID NO.46) |
| (Gly⁸,A6c³³,β-Ala³⁵)hGLP-1(7-36)NH₂; | (SEQ ID NO.47) |
| (Gly⁸,β-Ala^{22,35})hGLP-1(7-36)NH₂; | (SEQ ID NO.48) |
| (Gly⁸,Aib²²,(β-Ala³⁵)hGLP-1(7-36)NH₂; | (SEQ ID NO.49) |
| (Gly⁸,Glu²³,A6c³²,β-Ala³⁵)hGLP-1(7-36)NH₂; | (SEQ ID NO.50) |
| (Gly⁸,Aib²⁴,Glu²³,A6c³²,β-Ala³⁵)hGLP-1(7-36)NH₂; | (SEQ ID NO.51) |
| (Gly⁸,Aib²⁴,Glu²³,A6c³²,Lys³⁴(N^{e}octanoyl),β- | (SEQ ID NO.52) |
| Ala³⁵)hGLP-1(7-36)NH₂; | |
| (Gly⁸,Aib^{24,25},Glu²³,A6c³²,β-Ala³⁵)hGLP-1(7-36)NH₂; | (SEQ ID NO.53) |
| (Gly⁸,Aib^{24,25},A6c^{16,20,32},Glu²³,(β-Ala³⁵)hGLP-1(7-36)NH₂; | (SEQ ID NO.54) |
| (Gly⁸,Aib³⁵,D-Arg³⁶)hGLP-1(7-36)NH₂; | (SEQ ID NO.55) |
| (Gly⁸,Aib³⁵,D-Lys³⁵)hGLP-1(7-36)NH₂; | (SEQ ID NO.56) |
| (Gly⁸,β-Ala³⁵,D-Arg³⁶)hGLP-1(7-36)NH₂; | (SEQ ID NO.57) |
| (Gly⁸,β-Ala³⁵,D-Lys³⁶)hGLP-1(7-36)NH₂; | (SEQ ID NO.58) |
| (Gly⁸,Aib³⁵,Arg^{26,34})hGLP-1(7-36)NH₂; | (SEQ ID NO.59) |
| (Gly⁸,Arg^{26,34},β-Ala³⁵)hGLP-1(7-36)NH₂; | (SEQ ID NO.60) |
| (Gly⁸,Aib³⁵,Arg^{25,26,34})hGLP-1(7-36)NH₂; | (SEQ ID NO.61) |
| (Gly⁸,Arg^{25,26,34},β-Ala³⁵)hGLP-1(7-36)NH₂; | (SEQ ID NO.62) |
| (Gly⁸,Arg^{26,34},β-Ala³⁵,Lys³⁵(N^{ε}-tetradecanoyl))hGLP-1(7-36)OH; | (SEQ ID NO.63) |
| (Gly⁸,Aib³⁵,Arg^{26,34},Lys³⁶(N^{ε}-tetradecanoyl))hGLP-1(7-37)OH; | (SEQ ID NO.64) |
| (Gly⁸,Aib^{35,37},Arg^{26,34},Lys³⁶(N^{ε}-tetradecanoyl))hGLP-1(7-37)OH; | (SEQ ID NO.65) |
| (Gly⁸,Aib³⁵,Arg^{26,34},Lys³⁶(N^{ε}-tetradecanoyl),D | (SEQ ID NO.66) |
| Ala³⁷)hGLP-1(7-37)OH; | |
| (Gly⁸,Aib^{35,37},Arg^{26,34},Lys³⁸(N^{ε}-tetradecanoyl))hGLP-1(7-38)OH; | (SEQ ID NO.67) |
| (Gly⁸,Aib³⁵,Arg^{26,34},β-Ala³⁷,Lys³⁸(N^{ε}- | (SEQ ID NO.68) |
| tetradecanoyl))hGLP-1(7-38)OH; | |
| (Gly⁸,Aib³⁵,Arg^{26,34},Lys³⁸(N^{ε}-tetradecanoyl))hGLP-1(7-38)OH; | (SEQ ID NO.69) |
| (Gly⁸,Arg^{26,34},Lys³⁶(N^{ε}-tetradecanoyl),β-Ala³⁷)hGLP-1(7-37)OH; | (SEQ ID NO.70) |
| (Gly⁸,Aib³⁷,Arg^{26,34},Lys³⁶(N^{ε}-tetradecanoyl))hGLP-1(7-37)OH; | (SEQ ID NO.71) |
| (Gly⁸,Aib³⁵,Arg^{26,34},Ado³⁷)hGLP-1(7-37)OH; | (SEQ ID NO.72) |
| (Gly⁸,Aib³⁵,Arg^{26,34},Ado³⁷)hGLP-1(7-37)NH₂; | (SEQ ID NO.73) |
| (Gly⁸,Arg^{26,34},Lys³⁶(N^{ε}-tetradecanoyl),D-Ala³⁷)hGLP-1(7-37)OH; | (SEQ ID NO.74) |
| (Gly⁸,Aib³⁷,Arg^{26,34},Lys³⁸(N^{ε}-tetradecanoyl))hGLP-1(7-38)OH; | (SEQ ID NO.75) |
| (Gly⁸,Arg^{26,34},β-Ala³⁷,Lys³⁸(N^{ε}-tetradecanoyl))hGLP-1(7-38)OH; | (SEQ ID NO.76) |
| (Gly⁸,Aib³⁵,Lys²⁶(N^{ε}-octanoyl))hGLP-1(7-36)NH₂; | (SEQ ID NO.77) |
| (Gly⁸,Aib³⁵,Lys²⁶(N^{ε}-tetradecanoyl))hGLP-1(7-36)NH₂; | (SEQ ID NO.78) |
| (Gly⁸,Aib³⁵,Lys²⁶(N^{ε}-hexadecanoyI))hGLP-1(7-36)NH₂; | (SEQ ID NO.79) |
| (Gly⁸,Lys²⁶(N^{ε}-octanoyl),β-Ala³⁵)hGLP-1(7-36)NH₂; | (SEQ ID NO.80) |
| (Gly⁸,Lys²⁶(N^{ε}-tetradecanoyl),β-Ala³⁵)hGLP-1(7-36)NH₂; | (SEQ ID NO.81) |
| (Gly⁸,Lys²⁶(N^{ε}-hexadecanoyl),β-Ala³⁵)hGLP-1(7-36)NH₂; | (SEQ ID NO.82) |
| (Gly⁸,Aib³⁵,Lys²⁶(N^{ε}-octanoyl),Arg³⁴)hGLP-1(7-36)NH₂; | (SEQ ID NO.83) |
| (Gly⁸,Aib³⁵,Lys²⁶(N^{ε}-tetradecanoyl),Arg³⁴)hGLP-1(7-36)NH₂; | (SEQ ID NO.84) |
| (Gly⁸,Aib³⁵,Lys²⁶(N^{ε}-hexadecanoyl),Arg³⁴)hGLP-1(7-36)NH₂; | (SEQ ID NO.85) |
| (Gly⁸,Aib³⁵,Lys²⁶(N^{ε}-decanoyl),Arg³⁴)hGLP-1(7-36)NH₂; | (SEQ ID NO.86) |
| (Gly⁸,Aib³⁵,Lys²⁵,Lys²⁶(N^{ε}-octanoyl),Arg³⁴)hGLP-1(7-36)NH₂; | (SEQ ID NO.87) |
| (Gly⁸,Aib³⁵,Lys²⁵,Lys²⁶(N^{ε}-tetradecanoyl), | (SEQ ID NO.88) |
| Arg³⁴)hGLP-1(7-36)NH₂; | |
| (Gly⁸,Aib³⁵,Lys²⁵,Lys²⁶(N^{ε}-hexadecanoyl), | (SEQ ID NO.89) |
| Arg³⁴)hGLP-1(7-36)NH₂; | |
| (Gly⁸,Aib³⁵,Arg^{25,34},Lys²⁶(N^{ε}-octanoyl))hGLP-1(7-36)NH₂; | (SEQ ID NO.90) |
| (Gly⁸,Aib³⁵,Arg^{25,34},Lys²⁶(N^{ε}-tetradecanoyl))hGLP-1(7-36)NH₂; | (SEQ ID NO.91) |
| (Gly⁸,Aib³⁵,Arg^{25,34},Lys²⁶(N^{ε}-hexadecanoyl))hGLP-1(7-36)NH₂; | (SEQ ID NO.92) |
| (Gly⁸,Aib³⁵,Arg^{25,34},Lys²⁶(N^{ε}-decanoyl))hGLP-1(7-36)NH₂; | (SEQ ID NO.93) |
| (Gly⁸,Lys²⁶(N^{ε}-octanoyl),Arg³⁴,β-Ala³⁵)hGLP-1(7-36)NH₂; | (SEQ ID NO.94) |
| (Gly⁸,Lys²⁶(N^{ε}-tetradecanoyl),Arg³⁴,β-Ala³⁵)hGLP-1(7-36)NH₂; | (SEQ ID NO.95) |
| (Gly⁸,Lys²⁶(N^{ε}-hexadecanoyl),Arg³⁴,β-Ala³⁵)hGLP-1(7-36)NH₂; | (SEQ ID NO.96) |
| (Gly⁸,Lys²⁶(N^{ε}-decanoyl),Arg³⁴,β-Ala³⁵)hGLP-1(7-36)NH₂; | (SEQ ID NO.97) |
| (Gly⁸,Aib³⁵,Lys³⁴(N^{ε}-octanoyl))hGLP-1(7-36)NH₂; | (SEQ ID NO.98) |
| (Gly⁸,Aib³⁵,Lys³⁴(N^{ε}-tetradecanoyl))hGLP-1(7-36)NH₂; | (SEQ ID NO.99) |
| (Gly⁸,Aib³⁵,Lys³⁴(N^{ε}-hexadecanoyl))hGLP-1(7-36)NH₂; | (SEQ ID NO.100) |
| (Gly⁸,Aib³⁵,Arg²⁶,Lys³⁴(N^{ε}-octanoyl))hGLP-1(7-36)NH₂; | (SEQ ID NO.101) |
| (Gly⁸,Aib³⁵,Arg²⁶,Lys³⁴(N^{ε}-hexadecanoyl))hGLP-1(7-36)NH₂; | (SEQ ID NO.102) |
| (Gly⁸,Aib³⁵,Arg²⁶,Lys³⁴(N^{ε}-decanoyl))hGLP-1(7-36)NH₂; | (SEQ ID NO.103) |
| (Gly⁸,Aib³⁵,Arg^{25,26},Lys³⁴(N^{ε}-octanoyl))hGLP-1(7-36)NH₂; | (SEQ ID NO.104) |
| (Gly⁸,Aib³⁵,Arg^{25,26},Lys³⁴(N^{ε}-tetradecanoyl))hGLP-1(7-36)NH₂; | (SEQ ID NO.105) |
| (Gly⁸,Aib³⁵,Arg^{25,26},Lys³⁴(N^{ε}-hexadecanoyl))hGLP-1(7-36)NH₂; | (SEQ ID NO.106) |
| (Gly⁸,Aib³⁵,Arg^{25,26},Lys³⁴(N^{ε}-decanoyl))hGLP-1(7-36)NH₂; | (SEQ ID NO.107) |
| (Gly⁸,Aib³⁵,Lys²⁵,Arg²⁶,Lys³⁴(N^{ε}-octanoyl))hGLP-1(7-36)NH₂; | (SEQ ID NO.108) |
| (Gly⁸,Aib³⁵,Lys²⁵,Arg²⁶,Lys³⁴(N^{ε}- | (SEQ ID NO.109) |
| tetradecanoyl))hGLP-1(7-36)NH₂; | |
| (Gly⁸,Aib³⁵,Lys²⁵,Arg²⁶,Lys³⁴(N^{ε}- | (SEQ ID NO.110) |
| hexadecanoyl))hGLP-1(7-36)NH₂; | |
| (Gly⁸,Aib³⁵,Lys³⁶(N^{ε}-octanoyl))hGLP-1(7-36)NH₂; | (SEQ ID NO.111) |
| (Gly⁸,Aib³⁵,Lys³⁶(N^{ε}-tetradecanoyl))hGLP-1(7-36)NH₂; | (SEQ ID NO.112) |
| (Gly⁸,Aib³⁵,Lys³⁶(N^{ε}-hexadecanoyl))hGLP-1(7-36)NH₂; | (SEQ ID NO.113) |
| (Gly⁸,Aib³⁵,Arg²⁶,Lys³⁶(N^{ε}-octanoyl))hGLP-1(7-36)NH₂; | (SEQ ID NO.114) |
| (Gly⁸,Aib³⁵,Arg²⁶,Lys³⁶(N^{ε}-tetradecanoyl))hGLP-1(7-36)NH₂; | (SEQ ID NO.115) |
| (Gly⁸,Aib³⁵,Arg²⁶,Lys³⁶(N^{ε}-hexadecanoyl))hGLP-1(7-36)NH₂; | (SEQ ID NO.116) |
| (Gly⁸,Aib³⁵,Arg^{26,34},Lys³⁶(N^{ε}-octanoyl))hGLP-1(7-36)NH₂; | (SEQ ID NO.117) |
| (Gly⁸,Aib³⁵,Arg^{26,34},Lys³⁶(N^{ε}-hexadecanoyl))hGLP-1(7-36)NH₂; | (SEQ ID NO.118) |
| (Gly⁸,Aib³⁵,Arg^{26,34},Lys³⁸(N^{ε}-octanoyl))hGLP-1(7-38)NH₂; | (SEQ ID NO.119) |
| (Gly⁸,Aib³⁵,Arg^{26,34},Lys³⁸(N^{ε}-decanoyl))hGLP-1(7-38)NH₂; | (SEQ ID NO.120) |
| (Gly⁸,Aib³⁵,Arg^{26,34},Lys³⁸(N^{ε}-tetradecanoyl))hGLP-1(7-38)NH₂; | (SEQ ID NO.121) |
| (Gly⁸,Aib³⁵,Arg^{26,34},Lys³⁸(N^{ε}-hexadecanoyl))hGLP-1(7-38)NH₂; | (SEQ ID NO.122) |
| (Gly⁸,Aib^{35,37},Arg^{25,26,34},Lys³⁸(N^{ε}-octanoyl))hGLP-1(7-38)NH₂; | (SEQ ID NO.123) |
| (Gly⁸,Aib^{35,37},Arg^{25,26,34},Lys³⁸(N^{ε}-decanoyl))hGLP-1(7-38)NH₂; | (SEQ ID NO.124) |
| (Gly⁸,Aib^{35,37},Arg^{25,26,34},Lys³⁸(N^{ε}- | (SEQ ID NO.125) |
| tetradecanoyl))hGLP-1(7-38)NH₂; | |
| (Gly⁸,Aib^{35,37},Arg^{25,26,34},Lys³⁸(N^{ε}- | (SEQ ID NO.126) |
| hexadecanoyl))hGLP-1(7-38)NH₂; | |
| (Gly⁸,Aib^{35,37},Arg^{26,34},Lys³⁸(N^{ε}-octanoyl))hGLP-1(7-38)NH₂; | (SEQ ID NO.127) |
| (Gly⁸,Aib^{35,37},Arg^{26,34},Lys³⁸(N^{ε}-decanoyl))hGLP-1(7-38)NH₂; | (SEQ ID NO.128) |
| (Gly⁸,Aib^{35,37},Arg^{26,34},Lys³⁸(N^{ε}-hexadecanoyl))hGLP-1(7-38)NH₂; | (SEQ ID NO.129) |
| (Gly⁸,Aib³⁵,Lys²⁵,Arg^{26,34},Lys³⁶(N^{ε}-octanoyl))hGLP-1(7-36)NH₂; | (SEQ ID NO.130) |
| (Gly⁸,Aib³⁵,Lys²⁶,Arg^{26,34},Lys³⁶(N^{ε}- | (SEQ ID NO.131) |
| tetradecanoyl))hGLP-1(7-36)NH₂; | |
| (Gly⁸,Aib³⁵,Lys²⁵,Arg^{26,34},Lys³⁶(N^{ε}- | (SEQ ID NO.132) |
| hexadecanoyl))hGLP-1(7-36)NH₂; | |
| (Gly⁸,Aib³⁵,Arg^{25,26,34},Lys³⁶(N^{ε}-octanoyl))hGLP-1(7-36)NH₂; | (SEQ ID NO.133) |
| (Gly⁸,Aib³⁵,Arg^{25,26,34},Lys³⁶(N^{ε}-tetradecanoyl))hGLP-1(7-36)NH₂; | (SEQ ID NO.134) |
| (Gly⁸,Aib³⁵,Arg^{25,26,34},Lys³⁶(N^{ε}-hexadecanoyl))hGLP-1(7-36)NH₂; | (SEQ ID NO.135) |
| (Gly⁸,Aib³⁵,Arg^{25,26,34},Lys³⁶(N^{ε}-decanoyl))hGLP-1(7-36)NH₂; | (SEQ ID NO.136) |
| (Gly⁸,Lys³⁴(N^{ε}-octanoyl),β-Ala³⁵)hGLP-1(7-36)NH₂; | (SEQ ID NO.137) |
| (Gly⁸,Lys³⁴(N^{ε}-tetradecanoyl),β-Ala³⁵)hGLP-1(7-36)NH₂; | (SEQ ID NO.138) |
| (Gly⁸,Lys³⁴(N^{ε}-hexadecanoyt),β-Ala³⁵)hGLP-1(7-36)NH₂; | (SEQ ID NO.139) |
| (Gly⁸,A6c³²,Lys³⁴(N^{e}octanoyl),β-Ala³⁵)hGLP-1(7-36)NH₂; | (SEQ ID NO.140) |
| (Gly⁸,Glu²³,Lys³⁴(N^{e}octanoyl),β-Ala³⁵)hGLP-1(7-36)NH₂; | (SEQ ID NO.141) |
| (Gly⁸,Glu²³,A6c³²,Lys³⁴(N^{e}octanoyl),β-Ala³⁵)hGLP-1(7-36)NH₂; | (SEQ ID NO.142) |
| (Gly⁸,Arg²⁶,Lys³⁴(N^{ε}-octanoyl),β-Ala³⁵)hGLP-1(7-36)NH₂; | (SEQ ID NO.143) |
| (Gly⁸,Arg²⁶,Lys³⁴(N^{ε}-tetradecanoyl),β-Ala³⁵)hGLP-1(7-36)NH₂; | (SEQ ID NO.144) |
| (Gly⁸,Arg²⁶,Lys³⁴(N^{ε}-hexadecanoyl),β-Ala³⁵)hGLP-1(7-36)NH₂; | (SEQ ID NO.145) |
| (Gly⁸,Arg²⁶,Lys³⁴(N^{ε}-decanoyl),β-Ala³⁵)hGLP-1(7-36)NH₂; | (SEQ ID NO.146) |
| (Gly⁸,Arg^{25,26},Lys³⁴(N^{ε}-octanoyl),β-Ala³⁵)hGLP-1(7-36)NH₂; | (SEQ ID NO.147) |
| (Gly⁸,Arg^{25,26},Lys³⁴(N^{ε}-tetradecanoyl),β-Ala³⁵)hGLP-1(7-36)NH₂; | (SEQ ID NO.148) |
| (Gly⁸,Arg^{25,26},Lys³⁴(N^{ε}-hexadecanoyl),β-Ala³⁵)hGLP-1(7-36)NH₂; | (SEQ ID NO.149) |
| (Gly⁸,Arg^{25,26},Lys³⁴(N^{ε}-decanoyl),β-Ala³⁵)hGLP-1(7-36)NH₂; | (SEQ ID NO.150) |
| (Gly⁸,Lys²⁵,Arg²⁶,Lys³⁴(N^{ε}-octanoyl),β-Ala³⁵)hGLP-1(7-36)NH₂; | (SEQ ID NO.151) |
| (Gly⁸,Lys²⁵,Arg²⁶,Lys³⁴(N^{ε}-tetradecanoyl),β- | (SEQ ID NO.152) |
| Ala³⁵)hGLP-1(7-36)NH₂; | |
| (Gly⁸,Lys²⁵,Arg²⁶,Lys³⁴(N^{ε}-hexadecanoyl),β- | (SEQ ID NO.153) |
| Ala³⁵)hGLP-1(7-36)NH₂; | |
| (Gly⁸,β-Ala³⁵,Lys³⁶(N^{ε}-octanoyl))hGLP-1(7-36)NH₂; | (SEQ ID NO.154) |
| (Gly⁸,β-Ala³⁵,Lys³⁶(N^{ε}-tetradecanoyl))hGLP-1(7-36)NH₂; | (SEQ ID NO.155) |
| (Gly⁸,β-Ala³⁵,Lys³⁶(N^{ε}-hexadecanoyl))hGLP-1(7-36)NH₂; | (SEQ ID NO.156) |
| (Gly⁸,Arg²⁶,β-Ala³⁵,Lys³⁶(N^{ε}-octanoyl))hGLP-1(7-36)NH₂; | (SEQ ID NO.157) |
| (Gly⁸,Arg²⁶,β-Ala³⁵,Lys³⁶(N^{ε}-tetradecanoyl))hGLP-1(7-36)NH₂; | (SEQ ID NO.158) |
| (Gly⁸,Arg²⁶,β-Ala³⁵,Lys³⁶(N^{ε}-hexadecanoyl))hGLP-1(7-36)NH₂; | (SEQ ID NO.159) |
| (Gly⁸,Arg^{26,34},β-Ala³⁵,Lys³⁶(N^{ε}-octanoyl))hGLP-1(7-36)NH₂; | (SEQ ID NO.160) |
| (Gly⁸,Arg^{26,34},β-Ala³⁵,Lys³⁶(N^{ε}-tetradecanoyl))hGLP-1(7-36)NH₂; | (SEQ ID NO.161) |
| (Gly⁸,Arg^{26,34},β-Ala³⁵,Lys³⁵(N^{ε}-hexadecanoyl))hGLP-1(7-36)NH₂; | (SEQ ID NO.162) |
| (Gly⁸,Arg^{26,34},β-Ala³⁵,Lys³⁶(N^{ε}-decanoyl))hGLP-1(7-36)NH₂; | (SEQ ID NO.163) |
| (Gly⁸,Lys²⁵,Arg^{26,34},β-Ala³⁵,Lys³⁶(N^{ε}- | (SEQ ID NO.164) |
| octanoyl))hGLP-1(7-36)NH₂; | |
| (Gly⁸,Lys²⁵,Arg^{26,34},Lys³⁶(N^{ε}-tetradecanoyl),β | (SEQ ID NO.165) |
| Ala³⁵)hGLP-1(7-36)NH₂; | |
| (Gly⁸,Lys²⁵,Arg^{26,34},β-Ala³⁵,Lys³⁶(N^{ε}- | (SEQ ID NO.166) |
| hexadecanoyl))hGLP-1(7-36)NH₂; | |
| (Gly⁸,Arg^{25,26,34},β-Ala³⁵,Lys³⁶(N^{ε}-octanoyl))hGLP-1(7-36)NH₂; | (SEQ ID NO.167) |
| (Gly⁸,Arg^{25,26,34},β-Ala³⁵,Lys³⁶(N^{ε}- | (SEQ ID NO.168) |
| tetradecanoyl))hGLP-1(7-36)NH₂; | |
| (Gly⁸,Arg^{25,26,34},β-Ala³⁵,Lys³⁶(N^{ε}- | (SEQ ID NO.169) |
| hexadecanoyl))hGLP-1(7-36)NH₂; | |
| (Gly⁸,Arg^{25,26,34},β-Ala³⁵,Lys³⁶(N^{ε}-decanoyl))hGLP-1(7-36)NH₂; | (SEQ ID NO.170) |
| (Gly⁸,Aib³⁵,Lys²⁶(N^{ε}-octanoyl),A6c³²,Arg³⁴)hGLP-1(7-36)NH₂; | (SEQ ID NO.171) |
| (Gly⁸,Aib³⁵,Lys²⁶(N^{ε}-tetradecanoyl),A6c³², | (SEQ ID NO.172) |
| Arg³⁴)hGLP-1(7-36)NH₂; | |
| (Gly⁸,Aib³⁵,Lys²⁶(N^{ε}-hexadecanoyl),A6c³²,Arg³⁴)hGLP-1(7-36)NH₂; | (SEQ ID NO.173) |
| (Gly⁸,Aib³⁵,A6c³²,Lys³⁴(N^{ε}-octanoyl))hGLP-1(7-36)NH₂; | (SEQ ID NO.174) |
| (Gly⁸,Aib³⁵,A6c³²,Lys³⁴(N^{ε}-tetradecanoyl))hGLP-1(7-36)NH₂; | (SEQ ID NO.175) |
| (Gly⁸,Aib³⁵,A6c³²,Lys³⁴(N^{ε}-hexadecanoyl))hGLP-1(7-36)NH₂; | (SEQ ID NO.176) |
| (Gly⁸,Aib³⁵,Arg²⁶,A6c³²,Lys³⁴(N^{ε}-octanoyl))hGLP-1(7-36)NH₂; | (SEQ ID NO.177) |
| (Gly⁸,Aib³⁵,Arg²⁶,A6c³²,Lys³⁴(N^{ε}- | (SEQ ID NO.178) |
| tetradecanoyl))hGLP-1(7-36)NH₂; | |
| (Gly⁸,Aib³⁵,A6c³²,Lys³⁶(N^{ε}-octanoyl))hGLP-1(7-36)NH₂; | (SEQ ID NO.179) |
| (Gly⁸,Aib³⁵,A6c³²,Lys³⁶(N^{ε}-tetradecanoyl))hGLP-1(7-36)NH₂; | (SEQ ID NO.180) |
| (Gly⁸,Aib³⁵,A6c³²,Lys³⁶(N^{ε}-hexadecanoyl))hGLP-1(7-36)NH₂; | (SEQ ID NO.181) |
| (Gly⁸,Aib³⁵,Arg²⁶,A6c³²,Lys³⁶(N^{ε}-octanoyl))hGLP-1(7-36)NH₂; | (SEQ ID NO.182) |
| (Gly⁸,Aib³⁵,Arg²⁶,A6c³²,Lys³⁶(N^{ε}- | (SEQ ID NO.183) |
| tetradecanoyl))hGLP-1(7-36)NH₂; | |
| (Gly⁸,Aib³⁵,Arg²⁶,A6c³²,Lys³⁶(N^{ε}- | (SEQ ID NO.184) |
| hexadecanoyl))hGLP-1(7-36)NH₂; | |
| (Gly⁸,Aib³⁵,Arg^{26,34},A6c³²,Lys³⁶(N^{ε}-octanoyl))hGLP-1(7-36)NH₂; | (SEQ ID NO.185) |
| (Gly⁸,Aib³⁵,Arg^{26,34},A6c³²,Lys³⁶(N^{ε}-decanoyl))hGLP-1(7-36)NH₂; | (SEQ ID NO.186) |
| (Gly⁸,Aib³⁵,Arg^{26,34},A6c³²,Lys³⁶(N^{ε}- | (SEQ ID NO.187) |
| tetradecanoyl))hGLP-1(7-36)NH₂; | |
| (Gly⁸,Aib³⁵,Arg^{26,34},A6c³²,Lys³⁶(N^{ε}- | (SEQ ID NO.188) |
| hexadecanoyl))hGLP-1(7-36)NH₂; | |
| (Gly⁸,Aib^{24,35},Lys²⁶(N^{ε}-octanoyl),Arg³⁴)hGLP-1(7-36)NH₂; | (SEQ ID NO.189) |
| (Gly⁸,Aib^{24,35},Lys²⁶(N^{ε}-tetradecanoyl),Arg³⁴)hGLP-1(7-36)NH₂; | (SEQ ID NO.190) |
| (Gly⁸,Aib^{24,35},Lys²⁶(N^{ε}-hexadecanoyl),Arg³⁴)hGLP-1(7-36)NH₂; | (SEQ ID NO.191) |
| (Gly⁸,Aib^{24,35},Arg²⁶,Lys³⁴(N^{ε}-octanoyl))hGLP-1(7-36)NH₂; | (SEQ ID NO.192) |
| (Gly⁸,Aib^{24,35},Arg²⁶,Lys³⁴(N^{ε}-tetradecanoyl))hGLP-1(7-36)NH₂; | (SEQ ID NO.193) |
| (Gly⁸,Aib^{24,35},Arg²⁶,Lys³⁴(N^{ε}-hexadecanoyl))hGLP-1(7-36)NH₂; | (SEQ ID NO.194) |
| (Gly⁸,Aib^{24,35},Arg^{26,34},Lys³⁶(N^{ε}-octanoyl))hGLP-1(7-36)NH₂; | (SEQ ID NO.195) |
| (Gly⁸,Aib^{24,35},Arg^{26,34},Lys³⁶(N^{ε}-tetradecanoyl))hGLP-1(7-36)NH₂; | (SEQ ID NO.196) |
| (Gly⁸,Aib^{24,35},Arg^{26,34},Lys³⁶(N^{ε}-hexadecanoyl))hGLP-1(7-36)NH₂; | (SEQ ID NO.197) |
| (Gly⁸,Aib^{24,35},Glu²³,A6c³²,Lys³⁴(N^{e}octanoyl))hGLP-1(7-36)NH₂; | (SEQ ID NO.198) |
| (Gly⁸,Aib³⁵,Glu²³,Lys²⁶(N^{ε}-octanoyl),Arg³⁴)hGLP-1(7-36)NH₂; | (SEQ ID NO.199) |
| (Gly⁸,Aib³⁵,Glu²³,Lys²⁶(N^{ε}-tetradecanoyl), | (SEQ ID NO.200) |
| Arg³⁴)hGLP-1(7-36)NH₂; | |
| (Gly⁸,Aib³⁵,Glu²³,Lys²⁶(N^{ε}-hexadecanoyl), | (SEQ ID NO.201) |
| Arg³⁴)hGLP-1(7-36)NH₂; | |
| (Gly⁸,Aib³⁵,Glu²³,Lys³⁴(N^{e}octanoyl))hGLp-1(7-36)NH₂; | (SEQ ID NO.202) |
| (Gly⁸,Aib³⁵,Glu²³,A6c³²,Lys³⁴(N^{e}octanoyl))hGLP-1(7-36)NH₂; | (SEQ ID NO.203) |
| (Gly⁸,Aib³⁵,Glu²³,Arg²⁶,Lys³⁴(N^{ε}-octanoyl))hGLP-1(7-36)NH₂; | (SEQ ID NO.204) |
| (Gly⁸,Aib³⁵,Glu²³,Arg²⁶,Lys³⁴(N^{ε}- | (SEQ ID NO.205) |
| tetradecanoyl))hGLP-1(7-36)NH₂; | |
| (Gly⁸,Aib³⁵,Glu²³,Arg²⁶,Lys³⁴(N^{ε}- | (SEQ ID NO.206) |
| hexadecanoyl))hGLP-1(7-36)NH₂; | |
| (Gly⁸,Aib³⁵,Glu²³,Lys³⁶(N^{ε}-octanoyl))hGLP-1(7-36)NH₂; | (SEQ ID NO.207) |
| (Gly⁸,Aib³⁵,Glu²³,Lys³⁶(N^{ε}-tetradecanoyl))hGLP-1(7-36)NH₂; | (SEQ ID NO.208) |
| (Gly⁸,Aib³⁵,Glu²³,Lys³⁶(N^{ε}-hexadecanoyl))hGLP-1(7-36)NH₂; | (SEQ ID NO.209) |
| (Gly⁸,Aib³⁵,Glu²³,Arg^{26,34},Lys³⁶(N^{ε}-octanoyl))hGLP-1(7-36)NH₂; | (SEQ ID NO.210) |
| (Gly⁸,Aib³⁵,Glu²³,Arg^{26,34},Lys³⁶(N^{ε}- | (SEQ ID NO.211) |
| tetradecanoyl))hGLP-1(7-36)NH₂; | |
| (Gly⁸,Aib³⁵,Glu²³,Arg^{26,34},Lys³⁶(N^{ε}- | (SEQ ID NO.212) |
| hexadecanoyl))hGLP-1(7-36)NH₂; | |
| (Gly⁸,Aib^{30,35},Lys²⁶(N^{ε}-octanoyl),Arg³⁴)hGLP-1(7-36)NH₂; | (SEQ ID NO.213) |
| (Gly⁸,Aib^{30,35},Lys²⁶(N^{ε}-tetradecanoyl),Arg³⁴)hGLP-1(7-36)NH₂; | (SEQ ID NO.214) |
| (Gly⁸,Aib^{30,35},Lys²⁶(N^{ε}-hexadecanoyl),Arg³⁴)hGLP-1(7-36)NH₂; | (SEQ ID NO.215) |
| (Gly⁸,Aib^{30,35},Arg²⁶,Lys³⁴(N^{ε}-octanoyl))hGLP-1(7-36)NH₂; | (SEQ ID NO.216) |
| (Gly⁸,Aib^{30,35},Arg²⁶,Lys³⁴(N^{ε}-tetradecanoyl))hGLP-1(7-36)NH₂; | (SEQ ID NO.217) |
| (Gly⁸,Aib^{30,35},Arg²⁶,Lys³⁴(N^{ε}-hexadecanoyl))hGLP-1(7-36)NH₂; | (SEQ ID NO.218) |
| (Gly⁸,Aib^{30,35},Arg^{26,34},Lys³⁶(N^{ε}-octanoyl))hGLP-1(7-36)NH₂; | (SEQ ID NO.219) |
| (Gly⁸,Aib^{30,35},Arg^{26,34},Lys³⁶(N^{ε}-tetradecanoyl))hGLP-1(7-36)NH₂; | (SEQ ID NO.220) |
| (Gly⁸,Aib^{30,35},Arg^{26,34},Lys³⁶(N^{ε}-hexadecanoyl))hGLP-1(7-3a)NH₂; | (SEQ ID NO.221) |
| (Gly⁸,Aib³⁵,Glu²³,A6c³²,Lys³⁶(N^{ε}-octanoyl))hGLP-1(7-36)NH₂; | (SEQ ID NO.222) |
| (Gly⁸,Aib³⁵,Glu²³,A6c³²,Lys³⁶(N^{ε}- | (SEQ ID NO.223) |
| tetradecanoyl))hGLP-1(7-36)NH₂; | |
| (Gly⁸,Aib³⁵,Glu²³,A6c³²,Lys³⁶(N^{ε}- | (SEQ ID NO.224) |
| hexadecanoyl)hGLP-1(7-36)NH₂; | |
| (Gly⁸,Aib³⁵,Glu²³,Arg^{26,34},A6c³²,Lys³⁶(N^{ε}- | (SEQ ID NO.225) |
| octanoyl))hGLP-1(7-36)NH₂; | |
| (Gly⁸,Aib³⁵,Glu²³,Arg^{26,34},A6c³²,Lys³⁶(N^{ε}- | (SEQ ID NO.226) |
| tetradecanoyl))hGLP-1(7-36)NH₂; | |
| (Gly⁸,Aib³⁵,Glu²³,Arg^{26,34},A6c³²,Lys³⁶(N^{ε}- | (SEQ ID NO.227) |
| hexadecanoyl))hGLP-1(7-36)NH₂; | |
| (Gly⁸,Aib^{24,35},Glu²³,Arg^{26,34},A6c³²,Lys³⁶(N^{ε}- | (SEQ ID NO.228) |
| octanoyl))hGLP-1(7-36)NH₂; | |
| (Gly⁸,Aib^{24,35},Glu²³,Arg^{26,34},A6c³²,Lys³⁶(N^{ε}- | (SEQ ID NO.229) |
| tetradecanoyl))hGLP-1(7-36)NH₂; | |
| (Gly⁸,Aib^{24,35},Glu²³,Arg^{26,34},A6c³²,Lys³⁶(N^{ε}- | (SEQ ID NO.230) |
| hexadecanoyl))hGLP-1(7-36)NH₂; | |
| (Gly⁸,Aib^{24,30,35},Glu²³,Arg^{26,34},A6c³²,Lys³⁶(N^{ε}- | (SEQ ID NO.231) |
| octanoyl))hGLP-1(7-36)NH₂; | |
| (Gly⁸,Aib^{24,30,35},Glu²³,Arg^{26,34},A6c³²,Lys³⁵(N^{ε}- | (SEQ ID NO.232) |
| tetradecanoyl))hGLP-1(7-36)NH₂; | |
| (Gly⁸,Aib^{24,30,35},Glu²³,Arg^{26,34},A6c³²,Lys³⁶(N^{ε}- | (SEQ ID NO.233) |
| hexadecanoyl))hGLP-1(7-36)NH₂; | |
| ((N^{α}-HEPES-His)⁷,Gly⁸,β-Ala³⁵)hGLP-1(7-36)NH₂; | (SEQ ID NO.234) |
| ((N^{α}-HEPA-His)⁷,Gly⁸,β-Ala³⁵)hGLP-1(7-36)NH₂; | (SEQ ID NO.235) |
| ((N^{α}-tetradecanoyl-His)⁷,Gly⁸,Aib³⁵)hGLP-1(7-36)NH₂; | (SEQ ID NO.236) |
| ((N^{α}-tetradecanoyl-His)⁷,Gly⁸,β-Ala³⁵)hGLP-1(7-36)NH₂; | (SEQ ID NO.237) |
| ((N^{α}-tetradecanoyl-His)⁷,Gly⁸,Aib³⁵,Arg^{26,34})hGLP-1(7-36)NH₂; | (SEQ ID NO.238) |
| ((N^{α}-tetradecanoyl-His)⁷,Gly⁸,Arg^{26,34},β-Ala³⁵)hGLP-1(7-36)NH₂; | (SEQ ID NO.239) |
| ((N^{α}-tetradecanoyl-His)⁷,Gly⁸,Aib³⁵,Arg^{25,26,34})hGLP-1(7-36)NH₂; | (SEQ ID NO.240) |
| ((N^{α}-tetradecanoyl-His)⁷,Gly⁸,Arg^{25,26,34},β- | (SEQ ID NO.241) |
| Ala³⁵)hGLP-1(7-36)NH₂; | |
| (Gly⁸,Aib³⁵,Lys²⁶(N^{ε}-octanesulfonyl),Arg³⁴)hGLP-1(7-36)NH₂; | (SEQ ID NO.242) |
| (Gly⁸,Aib³⁵,Lys²⁶(N^{ε}-dodecanesulfonyl),Arg³⁴)hGLP-1(7-36)NH₂; | (SEQ ID NO.243) |
| (Gly⁸,Aib³⁵,Lys²⁶(N^{ε}-hexadecanesulfonyl), | (SEQ ID NO.244) |
| Arg³⁴)hGLP-1(7-36)NH₂; | |
| (Gly⁸,Aib³⁵,Arg²⁶,Lys³⁴(N^{ε}-octanesulfonyl))hGLP-1(7-36)NH₂; | (SEQ ID NO.245) |
| (Gly⁸,Aib³⁵,Arg²⁶,Lys³⁴(N^{ε}-dodecanesulfonyl))hGLP-1(7-36)NH₂; | (SEQ ID NO.246) |
| (Gly⁸,Aib³⁵,Arg²⁶,Lys³⁴(N^{ε}-hexadecanesulfonyl)) | (SEQ ID NO.247) |
| hGLP-1(7-36)NH₂; | |
| (Gly⁸,Aib³⁵,Arg^{26,34},Lys³⁶(N^{ε}-octanesulfonyl))hGLP-1(7-36)NH₂; | (SEQ ID NO.248) |
| (Gly⁸,Aib³⁵,Arg^{26,34},Lys³⁶(N^{ε}- | (SEQ ID NO.249) |
| hexadecanesulfonyl))hGLP-1(7-36)NH₂; | |
| (Gly⁸,Aib³⁵,Asp²⁶(1-(4-decylpiperazine)),Arg³⁴)hGLP-1(7-36)NH₂; | (SEQ ID NO.250) |
| (Gly⁸,Aib³⁵,Asp²⁶(1-(4-dodecylpiperazine)), | (SEQ ID NO.251) |
| Arg³⁴)hGLP-1(7-36)NH₂; | |
| (Gly⁸,Aib³⁵,Asp²⁶(1 -(4-tetradecylpiperazine)), | (SEQ ID NO.252) |
| Arg³⁴)hGLP-1(7-36)NH₂; | |
| (Gly⁸,Aib³⁵,Asp²⁶(1 -(4-hexadecylpiperazine)), | (SEQ ID NO.253) |
| Arg³⁴)hGLP-1(7-36)NH₂; | |
| (Gly⁸,Aib³⁵,Arg²⁶,Asp³⁴(1-(4-decylpiperazine)))hGLP-1(7-36)NH₂; | (SEQ ID NO.254) |
| (Gly⁸,Aib³⁵,Arg²⁶,Asp³⁴(1-(4- | (SEQ ID NO.255) |
| dodecylpiperazine)))hGLP-1(7-36)NH₂; | |
| (Gly⁸,Aib³⁵,Arg²⁶1,Asp³⁴(1-(4- | (SEQ ID NO.256) |
| tetradecylpiperazine)))hGLP-1(7-36)NH₂; | |
| (Gly⁸,Aib³⁵,Arg²⁶,Asp³⁴(1-(4- | (SEQ ID NO.257) |
| hexadecylpiperazine)))hGLP-1(7-36)NH₂; | |
| (Gly⁸,Aib³⁵,Arg^{26,34},Asp³⁶(1-(4- | (SEQ ID NO.258) |
| decylpiperazine)))hGLP-1(7-36)NH₂; | |
| (Gly⁸,Aib³⁵,Arg^{26,34},Asp³⁶(1-(4- | (SEQ ID NO.259) |
| doclecylpiperazine)))hGLP-1(7-36)NH₂; | |
| (Gly⁸,Aib³⁵,Arg^{26,34},Asp³⁶(1-(4- | (SEQ ID NO.260) |
| hexadecylpiperazine)))hGLP-1(7-36)NH₂; | |
| (Gly⁸,Aib³⁵,Arg^{26,34},Asp³⁸(1-(4- | (SEQ ID NO.261) |
| decylpiperazine)))hGLP-1(7-38)NH₂; | |
| (Gly⁸,Aib³⁵,Arg^{26,34},Asp³⁸(1-(4- | (SEQ ID NO.262) |
| dodecylpiperazine)))hGLP-1(7-38)NH₂; | |
| (Gly⁸,Aib³⁵,Arg^{26,34},Asp³⁸(1-(4- | (SEQ ID NO.263) |
| tetradecylpiperazine)))hGLP-1(7-38)NH₂; | |
| (Gly⁸,Aib³⁵,Arg^{26,34},Asp³⁸(1-(4- | (SEQ ID NO.264) |
| hexadecylpiperazine)))hGLP-1(7-38)NH₂; | |
| (Gly⁸,Aib^{35,37},Arg^{26,34},Asp³⁸(1-(4- | (SEQ ID NO.265) |
| decylpiperazine)))hGLP-1(7-38)NH₂; | |
| (Gly⁸,Aib^{35,37},37,Arg^{26,34},Asp³⁸(1-(4- | (SEQ ID NO.266) |
| dodecylpiperazine)))hGLP-1(7-38)NH₂; | |
| (Gly⁸,Aib^{35,37},Arg^{26,34},Asp³⁸(1-(4- | (SEQ ID NO.267) |
| tetradecylpiperazine)))hGLP-1(7-38)NH₂; | |
| (Gly⁸,Aib^{35,37},Arg^{26,34},Asp³⁸(1-(4- | (SEQ ID NO.268) |
| hexadecylpiperazine)))hGLP-1(7-38)NH₂; | |
| (Gly⁸,Aib³⁵,Arg^{25,34},Asp²⁶(1-(4- | (SEQ ID NO.269) |
| decylpiperazine)))hGLP-1(7-36)NH₂; | |
| (Gly⁸,Aib³⁵,Arg^{25,34},Asp²⁶(1-(4- | (SEQ ID NO.270) |
| dodecylpiperazine)))hGLP-1(7-36)NH₂; | |
| (Gly⁸,Aib³⁵,Arg^{25,34},Asp²⁶(1-(4- | (SEQ ID NO.271) |
| tetradecylpiperazine)))hGLP-1(7-36)NH₂; | |
| (Gly⁸,Aib³⁵,Arg^{25,34},Asp²⁶(1-(4- | (SEQ ID NO.272) |
| hexadecylpiperazine)))hGLP-1(7-36)NH₂; | |
| (Gly⁸,Aib³⁵,Arg^{25,26},Asp³⁴(1-(4- | (SEQ ID NO.273) |
| decylpiperazine)))hGLP-1(7-36)NH₂; | |
| (Gly⁸,Aib³⁵,Arg^{25,26},Asp³⁴(1-(4- | (SEQ ID NO.274) |
| dodecylpiperazine)))hGLP-1(7-36)NH₂; | |
| (Gly⁸,Aib³⁵,Arg^{25,26},Asp³⁴(1-(4- | (SEQ ID NO.275) |
| tetradecylpiperazine)))hGLP-1(7-36)NH₂; | |
| (Gly⁸,Aib³⁵,Arg^{25,26},Asp³⁴(1-(4- | (SEQ ID NO.276) |
| hexadecylpiperazine)))hGLP-1(7-36)NH₂; | |
| (Gly⁸,Aib³⁵,Arg^{25,26,34},Asp³⁶(1-(4- | (SEQ ID NO.277) |
| decylpiperazine)))hGLP-1(7-36)NH₂; | |
| (Gly⁸,Aib³⁵,Arg^{25,26,34},Asp³⁶(1-(4- | (SEQ ID NO.278) |
| dodecylpiperazine)))hGLP-1(7-36)NH₂; | |
| (Gly⁸,Aib³⁵,Arg^{25,26,34},Asp³⁶(1-(4- | (SEQ ID NO.279) |
| tetradecylpiperazine)))hGLP-1(7-36)NH₂; | |
| (Gly⁸,Aib³⁵,Arg^{25,26,34},Asp³⁶(1-(4- | (SEQ ID NO.280) |
| hexadecylpiperazine)))hGLP-1(7-36)NH₂; | |
| (Gly⁸,Aib³⁵,Arg^{25,26,34},Asp³⁸(1-(4- | (SEQ ID NO.281) |
| decylpiperazine)))hGLP-1(7-38)NH₂; | |
| (Gly⁸,Aib³⁵,Arg^{25,26,34},Asp³⁸(1-(4- | (SEQ ID NO.282) |
| dodecylpiperazine)))hGLP-1(7-38)NH₂; | |
| (Gly⁸,Aib³⁵,Arg^{25,26,34},Asp³⁸(1-(4- | (SEQ ID NO.283) |
| tetradecylpiperazine)))hGLP-1(7-38)NH₂; | |
| (Gly⁸,Aib³⁵,Arg^{25,26,34},Asp³⁸(1-(4- | (SEQ ID NO.284) |
| hexadecylpiperazine)))hGLP-1(7-38)NH₂; | |
| (Gly⁸,Aib^{35,37},Arg^{25,26,34},Asp³⁸(1-(4- | (SEQ ID NO.285) |
| decylpiperazine)))hGLP-1(7-38)NH₂; | |
| (Gly⁸,Aib^{35,37},Arg^{25,26,34},Asp³⁸(1-(4- | (SEQ ID NO.286) |
| dodecyipiperazine)))hGLP-1(7-38)NH₂; | |
| (Gly⁸,Aib^{35,37},Arg^{25,26,34},Asp³⁸(1-(4- | (SEQ ID NO.287) |
| tetradecylpiperazine)))hGLP-1(7-38)NH₂; | |
| (Gly⁸,Aib^{35,37},Arg^{25,26,34},Asp³⁸(1-(4- | (SEQ ID NO.288) |
| hexadecylpiperazine)))hGLP-1(7-38)NH₂; | |
| (Gly⁸,Aib³⁵,Arg^{26,34},Glu³⁶(1-dodecylamino))hGLP-1(7-36)NH₂; | (SEQ ID NO.289) |
| (Gly⁸,Aib³⁵,Glu²⁶(1-dodecylamino),Arg³⁴)hGLP-1(7-36)NH₂; | (SEQ ID NO.290) |
| (Gly⁸,Aib³⁵,Arg²⁶,Glu³⁴(1-dodecylamino))hGLP-1(7-36)NH₂; | (SEQ ID NO.291) |
| (Gly⁸,Aib^{35,37},Arg^{26,34},Glu³⁸(1-dodecylamino))hGLP-1(7-38)NH₂; | (SEQ ID NO.292) |
| (Gly⁸,Aib³⁵,Arg³⁴,Lys²⁶(N^{ε}-(2-(4-decyl-1-piperazine)- | (SEQ ID NO.293) |
| acetyl)))hGLP-1(7-36)NH₂; | |
| (Gly⁸,Aib³⁵,Arg³⁴,Lys²⁶(N^{ε}-(2-(4-dodecyl-1-piperazine)- | (SEQ ID NO.294) |
| acetyl)))hGLP-1(7-36)NH₂; | |
| (Gly⁸,Aib³⁵,Arg³⁴,Lys²⁶(N^{ε}-(2-(4-tetradecyl-1-piperazine)- | (SEQ ID NO.295) |
| acetyl)))hGLP-1(7-36)NH₂; | |
| (Gly⁸,Aib³⁵,Arg³⁴,Lys²⁶(N^{ε}-(2-(4-hexadecyl-1-piperazine)- | (SEQ ID NO.296) |
| acetyl)))hGLP-1(7-36)NH₂; | |
| (Gly⁸,Aib³⁵,Arg²⁶,Lys³⁴(N^{ε}-(2-(4-decyl-1-piperazine)- | (SEQ ID NO.297) |
| acetyl)))hGLP-1(7-36)NH₂; | |
| (Gly⁸,Aib³⁵,Arg²⁶,Lys³⁴(N^{ε}-(2-(4-dodecyl-1-piperazine)- | (SEQ ID NO.298) |
| acetyl))hGLP-1(7-36)NH₂; | |
| (Gly⁸,Aib³⁵,Arg²⁶,Lys³⁴(N^{ε}-(2-(4-tetradecyl-1-piperazine)- | (SEQ ID NO.299) |
| acetyl)))hGLP-1(7-36)NH₂; | |
| (Gly⁸,Aib³⁵,Arg²⁶,Lys³⁴(N^{ε}-(2-(4-hexadecyl-1-piperazine) | (SEQ ID NO.300) |
| acetyl)))hGLP-1(7-36)NH₂; | |
| (Gly⁸,Aib³⁵,Arg^{26,34},Lys³⁶(N^{ε}-(2-(4-hexadecyl-1-piperazine)- | (SEQ ID NO.301) |
| acetyl)))hGLP-1(7-36)NH₂; | |
| (Gly⁸,Aib³⁵,Arg^{26,34},Lys³⁶(N^{ε}-(2-(4-dodecyl-1-piperazine)- | (SEQ ID NO.302) |
| acetyl)))hGLP-1(7-36)NH₂; | |
| (Gly⁸,Aib³⁵,Arg^{26,34},Lys³⁶(N^{ε}-(2-(4-hexadecyl-1-piperazine)- | (SEQ ID NO.303) |
| acetyl)))hGLP-1(7-36)NH₂; | |
| (Gly⁸,Aib³⁵,Arg^{26,34},Lys³⁸(N^{ε}-(2-(4-decyl-1-piperazine)- | (SEQ ID NO.304) |
| acetyl)))hGLP-1(7-38)NH₂; | |
| (Gly⁸,Aib³⁵,Arg^{26,34},Lys³⁸(N^{ε}-(2-(4-dodecyl-1-piperazine) | (SEQ ID NO.305) |
| acetyl)))hGLP-1(7-38)NH₂; | |
| (Gly⁸,Aib³⁵,Arg^{26,34},Lys³⁸(N^{ε}-(2-(4-tetradecyl-1-piperazine)- | (SEQ ID NO.306) |
| acetyl)))hGLP-1(7-38)NH₂; | |
| (Gly⁸,Aib³⁵,Arg^{26,34},Lys³⁸(N^{ε}-(2-(4-hexadecyl-1-piperazine)- | (SEQ ID NO.307) |
| acetyl)))hGLP-1(7-38)NH₂; | |
| (Gly⁸,Aib^{35,37},Arg^{26,34},Lys³⁸(N^{ε}-(2-(4-decyl-1-piperazine) | (SEQ ID NO.308) |
| acetyl)))hGLP-1(7-38)NH₂; | |
| (Gly⁸,Aib^{35,37},Arg^{26,34},Lys³⁸(N^{ε}-(2-(4-dodecyl-1-piperazine)- | (SEQ ID NO.309) |
| acetyl)))hGLP-1(7-38)NH₂; | |
| (Gly⁸,Aib^{35,37},Arg^{26,34},Lys³⁸(N^{ε}-(2-(4-tetradecyl-1-piperazine)- | (SEQ ID NO.310) |
| acetyl)))hGLP-1(7-38)NH₂; | |
| (Gly⁸,Aib^{35,37},Arg^{26,34},Lys³⁸(N^{ε}-(2-(4-hexadecyl-1-piperazine) | (SEQ ID NO.311) |
| acetyl)))hGLP-1(7-38)NH₂; | |
| (Gly⁸,Aib³⁵,Arg^{25,34},Lys²⁶(N^{ε}-(2-(4-decyl-1-piperazine)- | (SEQ ID NO.312) |
| acetyl)))hGLP-1(7-36)NH₂; | |
| (Gly⁸,Aib³⁵,Arg^{25,34},Lys²⁶(N^{ε}-(2-(4-dodecyl-1-piperazine)- | (SEQ ID NO.313) |
| acetyl)))hGLP-1(7-36)NH₂; | |
| (Gly⁸,Aib³⁵,Arg^{25,34},Lys²⁶(N^{ε}-(2-(4-tetradecyl-1-piperazine) | (SEQ ID NO.314) |
| acetyl)))hGLP-1(7-36)NH₂; | |
| (Gly⁸,Aib³⁵,Arg^{25,34},Lys²⁶(N^{ε}-(2-(4-hexadecyl-1-piperazine) | (SEQ ID NO.315) |
| acetyl)))hGLP-1(7-36)NH₂; | |
| (Gly⁸,Aib³⁵,Arg^{25,26},Lys³⁴(N^{ε}-(2-(4-decyl-1-piperazine)- | (SEQ ID NO.316) |
| acetyl)))hGLP-1(7-36)NH₂; | |
| (Gly⁸,Aib³⁵,Arg^{25,26},Lys³⁴(N^{ε}-(2-(4-dodecyl-1-piperazine)- | (SEQ ID NO.317) |
| acetyl)))hGLP-1(7-36)NH₂; | |
| (Gly⁸,Aib³⁵,Arg^{25,26},Lys³⁴(N^{ε}-(2-(4-tetradecyl-1-piperazine) | (SEQ ID NO.318) |
| acetyl)))hGLP-1(7-36)NH₂; | |
| (Gly⁸,Aib³⁵,Arg^{25,26},Lys³⁴(N^{ε}-(2-(4-hexadecyl-1-piperazine) | (SEQ ID NO.319) |
| acetyl)))hGLP-1(7-36)NH₂; | |
| (Gly⁸,Aib³⁵,Arg^{25,26,34},Lys³⁶(N^{ε}-(2-(4-decyl-1-piperazine) | (SEQ ID NO.320) |
| acetyl)))hGLP-1(7-36)NH₂; | |
| (Gly⁸,Aib³⁵,Arg^{25,26,34},Lys³⁶(N^{ε}-(2-(4-dodecyl-1-piperazine) | (SEQ ID NO.321) |
| acetyt)))hGLP-1(7-36)NH₂; | |
| (Gly⁸,Aib³⁵,Arg^{25,26,34},Lys³⁶(N^{ε}-(2-(4-tetradecyl-1-piperazine) | (SEQ ID NO.322) |
| acetyl)))hGLP-1(7-36)NH₂; | |
| (Gly⁸,Aib³⁵,Arg^{25,26,34},Lys³⁶(N^{ε}-(2-(4-hexadecyl-1-piperazine) | (SEQ ID NO.323) |
| acetyl)))hGLP-1(7-36)NH₂; | |
| (Gly⁸,Aib³⁵,Arg^{25,26,34},Lys³⁸(N^{ε}-(2-(4-decyl-1-piperazine)- | (SEQ ID NO.324) |
| acetyl)))hGLP-1(7-38)NH₂; | |
| (Gly⁸,Aib³⁵,Arg^{25,26,34},Lys³⁸(N^{ε}-(2-(4-dodecyl-1-piperazine)- | (SEQ ID NO.325) |
| acetyl)))hGLP-1(7-38)NH₂; | |
| (Gly⁸,Aib³⁵,Arg^{25,26,34},Lys³⁸(N^{ε}-(2-(4-tetradecyl-1-piperazine) | (SEQ ID NO.326) |
| acetyl)))hGLP-1(7-38)NH₂; | |
| (Gly⁸,Aib³⁵,Arg^{25,26,34},Lys³⁹(N^{ε}-(2-(4-hexadecyl-1-piperazine) | (SEQ ID NO.327) |
| acetyl)))hGLP-1(7-38)NH₂; | |
| (Gly⁸,Aib^{35,37},Arg^{25,26,34},Lys³⁸(N^{ε}-(2-(4-decyl-1-piperazine) | (SEQ ID NO.328) |
| acetyl)))hGLP-1(7-38)NH₂; | |
| (Gly⁸,Aib^{35,37},Arg^{25,26,34},Lys³⁸(N^{ε}-(2-(4-dodecyl-1-piperazine) | (SEQ ID NO.329) |
| acetyl)))hGLP-1(7-38)NH₂; | |
| (Gly⁸,Aib^{35,37},Arg^{25,26,34},Lys³⁸(N^{ε}-(2-(4-tetradecyl-1-piperazine)- | (SEQ ID NO.330) |
| acetyl)))hGLP-1(7-38)NH₂; | |
| (Gly⁸,Aib^{35,37},Arg^{25,26,34},Lys³⁸(N^{ε}-(2-(4-hexadecyl-1-piperazine) | (SEQ ID NO.331) |
| acetyl)))hGLP-1(7-38)NH₂; | |
| (Gly⁸,Aib³⁵,Arg^{26,34},Lys³⁶(N^{ε}-decanoyl))hGLP-1(7-36)OH; | (SEQ ID NO.332) |
| (Gly⁸,Aib³⁵,Lys²⁵,Arg^{26,34},Lys³⁶(N^{ε}-decanoyl))hGLP-1(7-36)OH; | (SEQ ID NO.333) |
| (Gly⁸,Aib³⁵,Arg^{26,34},Ava³⁷,Ado³⁸)hGLP-1(7-38)NH₂; | (SEQ ID NO.334) |
| (Gly⁸,Aib³⁵,Arg^{26,34},Asp³⁷,Ava³⁶,Ado³⁹)hGLP-1(7-39)NH₂; | (SEQ ID NO.335) |
| (Gly⁸,Aib³⁵,Arg^{26,34},Aun³⁷)hGLP-1(7-37)NH₂; | (SEQ ID NO.336) |
| (Gly⁸,Aib^{17,35})hGLP-1(7-36)NH₂; | (SEQ ID NO.337) |
| (Gly⁸,Arg^{26,34},β-Ala³⁵,D-Asp³⁷,Ava³⁸,Aun³⁹)hGLP-1(7-39)NH₂; | (SEQ ID NO.338) |
| (Gly⁸,Glu^{22,23},β-Ala³⁵)hGLP-1(7-36)NH₂; | (SEQ ID NO.339) |
| (Gly⁸,Lys¹⁸,β-Ala³⁵)hGLP-1(7-36)NH₂; | (SEQ ID NO.340) |
| (Gly⁸,Leu²⁷,β-Ala³⁵)hGLP-1(7-36)NH₂; | (SEQ ID NO.341) |
| (Gly⁸,Lys³³,β-Ala³⁵)hGLP-1(7-36)NH₂; | (SEQ ID NO.342) |
| (Gly⁸,Lys¹⁸,Leu²⁷,(β-Ala³⁵)hGLP-1(7-36)NH₂; | (SEQ ID NO.343) |
| (Gly⁸,D-Arg³⁶)hGLP-1(7-36)NH₂; | (SEQ ID NO.344) |
| (Gly⁸,β-Ala³⁵,D-Arg³⁷)hGLP-1(7-37)NH₂; | (SEQ ID NO.345) |
| (Gly⁸,Aib²⁷,β-Ala³⁵)hGLP-1(7-36)NH₂; | (SEQ ID NO.346) |
| (Gly⁸,Aib²⁷,β-Ala^{35,37},Arg³⁸)hGLP-1(7-38)NH₂; | (SEQ ID NO.347) |
| (Gly⁸,Aib²⁷,(β-Ala^{35,37},Arg^{38,39})hGLP-1(7-39)NH₂; | (SEQ ID NO.348) |
| (Gly⁸,Lys^{18,27},β-Ala³⁵)hGLP-1(7-36)NH₂; | (SEQ ID NO.349) |
| (Gly⁸,Lys²⁷,β-Ala³⁵)hGLP-1(7-36)NH₂; | (SEQ ID NO.350) |
| (Gly⁸,β-Ala³⁵,Arg³⁸)hGLP-1(7-38)NH₂; | (SEQ ID NO.351) |
| (Gly⁸,Arg^{26,34},β-Ala³⁵,)hGLP-1(7-36)NH₂; | (SEQ ID NO.352) |
| (Gly⁸,D-Arg³⁵)hGLP-1(7-36)NH₂; | (SEQ ID NO.353) |
| (Gly⁸,β-Ala³⁵,Arg³⁷)hGLP-1(7-37)NH₂; | (SEQ ID NO.354) |
| (Gly⁸,Phe³¹,β-Ala³⁵)hGLP-1(7-36)NH₂; | (SEQ ID NO.355) |
| (Gly⁸,Aib³⁵,Phe³¹)hGLP-1(7-36)NH₂; | (SEQ ID NO.356) |
| (Gly⁸,Aib³⁵,2-Nal³¹)hGLP-1(7-36)NH₂; | (SEQ ID NO.357) |
| (Gly⁸,Aib³⁵,2-Nal^{28,31})hGLP-1(7-36)NH₂; | (SEQ ID NO.358) |
| (Gly⁸,Aib³⁵,Arg^{26,34},2-Nal³¹)hGLP-1(7-36)NH₂; | (SEQ ID NO.359) |
| (Gly⁸,Aib³⁵,Arg^{26,34},Phe³¹)hGLP-1(7-36)NH₂; | (SEQ ID NO.360) |
| (Gly⁸,Aib³⁵,2-Nal^{19,31})hGLP-1(7-36)NH₂; | (SEQ ID NO.361) |
| (Gly⁸,Aib³⁵,2-Nal^{12,31})hGLP-1(7-36)NH₂; | (SEQ ID NO.362) |
| (Gly⁸,Aib³⁵,Lys³⁶(N^{ε}-decanoyl))hGLP-1(7-36)NH₂; | (SEQ ID NO.363) |
| (Gly⁸,Aib³⁵,Arg³⁴,Lys²⁶(N^{ε}-decanoyl))hGLP-1(7-36)NH₂; | (SEQ ID NO.364) |
| (Gly⁸,Aib³⁵,Arg^{26,34},Lys³⁶(N^{ε}-dodecanoyl))hGLP-1(7-36)NH₂; | (SEQ ID NO.365) |
| (Gly⁸,β-Ala³⁵,Ser³⁷(O-decanoyl))hGLP1(7-37)-NH₂; | (SEQ ID NO.366) |
| (Gly⁸,Aib²⁷,β-Ala^{35,37},Arg³⁸,Lys³⁹(N^{ε}-octanoyl))hGLP-1(7-39)NH₂; | (SEQ ID NO.367) |
| (Gly⁸,Arg^{26,34},β-Ala³⁵,Lys³⁷(N^{ε}-octanoyl))hGLP-1(7-37)NH₂; | (SEQ ID NO.368) |
| (Gly⁸,Arg^{26,34},β-Ala³⁵,Lys³⁷(N^{ε}-decanoyl))hGLP-1(7-37)NH₂; | (SEQ ID NO.369) |
| (Gly⁸,Arg^{26,34},β-Ala³⁵,Lys³⁷(N^{ε}-tetradecanoyl))hGLP-1(7-37)NH₂; | (SEQ ID NO.370) |
| (Gly⁸,Arg^{26,34},β-Ala³⁵,Lys³⁷(N^{ε}-dodecanoyl))hGLP-1(7-37)NH₂; | (SEQ ID NO.371) |
| (Gly⁸,Arg^{26,34},β-Ala³⁵,Lys³⁷(N^{ε}-dodecanoyl))hGLP-1(8-37)NH₂; | (SEQ ID NO.372) |
| (Gly⁸,Arg²⁶,Aib³⁵)hGLP-1(7-36)NH₂; | (SEQ ID NO.373) |
| (Gly⁸,Arg²⁶,Phe³¹,Aib³⁵)hGLP-1(7-36)NH₂; | (SEQ ID NO.374) |
| (Gly⁸,Arg²⁶,Phe³¹,β-Ala³⁵)hGLP-1(7-36)NH₂; | (SEQ ID NO.375) |
| (Gly⁸,Arg²⁶,β-Ala³⁵)hGLP-1(7-36)NH₂; | (SEQ ID NO.376) |
| (Gly⁸,Arg^{26,34},Phe³¹,Aib³⁵)hGLP-1(7-36)NH₂; | (SEQ ID NO.377) |
| (Gly⁸,Arg^{26,34},Phe³¹,β-Ala³⁵)hGLP-1(7-36)NH₂; | (SEQ ID NO.378) |
| (Gly⁸,Arg³⁴,Aib³⁵)hGLP-1(7-36)NH₂; | (SEQ ID NO.379) |
| (Gly⁸,Arg³⁴,β-Ala³⁵)hGLP-1(7-36)NH₂; | (SEQ ID NO.380) |
| (Gly⁸,Phe³¹,Arg³⁴,Aib³⁵)hGLP-1(7-36)NH₂; | (SEQ ID NO.381) |
| (Gly⁸,Phe³¹,Arg³⁴,β-Ala³⁵)hGLP-1(7-36)NH₂; | (SEQ ID NO.382) |
| (Gly⁸,Arg^{26,34},β-Ala³⁵,Lys³⁸(N^{ε}-Aec-decanoyl))hGLP-1(7-36)NH₂; | (SEQ ID NO.383) |
| or | |
| (Gly⁸,β-Ala³⁵,Ser³⁷(O-decanoyl))hGLP1(7-37)-NH₂; | (SEQ ID NO.384) |

or a pharmaceutically acceptable salt thereof.

Another preferred compound of the group of compounds described in paragraph [067] is a compound according to the formula:

| | |
|---|---|
| ((N^{α}-Me-His)⁷,Ser⁸,Aib³⁵)hGLP-1(7-36)NH₂; | (SEQ ID NO.385) |
| ((N^{α}-Me-His)⁷,Ser⁸,β-Ala³⁵)hGLP-1(7-36)NH₂; | (SEQ ID NO.386) |
| ((N^{α}-Me-His)⁷,Ser⁸,Aib³⁵,Arg^{26,34})hGLP-1(7-36)NH₂; | (SEQ ID NO.387) |
| ((N^{α}-Me-His)⁷,Ser⁸,Arg^{26,34},β-Ala³⁵)hGLP-1(7-36)NH₂; | (SEQ ID NO.388) |
| (Ser⁸,A6c³⁵)hGLP-1(7-36)NH₂; | (SEQ ID NO.389) |
| (Ser⁸,A5c³⁵)hGLP-1(7-36)NH₂; | (SEQ ID NO.390) |
| (Ser⁸,D-Ala³⁵)hGLP-1(7-36)NH₂; | (SEQ ID NO.391) |
| (Ser⁸,Aib³⁵,A6c³²)hGLP-1(7-36)NH₂; | (SEQ ID NO.392) |
| (Ser⁸,Aib³⁵,A5c³²)hGLP-1(7-36)NH₂; | (SEQ ID NO.393) |
| (Ser⁸,Aib³⁵,Glu²³)hGLP-1(7-36)NH₂; | (SEQ ID NO.394) |
| (Ser⁸,Aib^{24,35})hGLP-1(7-36)NH₂; | (SEQ ID NO.395) |
| (Ser⁸,Aib^{30,35})hGLP-1(7-36)NH₂; | (SEQ ID NO.396) |
| (Ser⁸,Aib^{25,35})hGLP-1(7-36)NH₂; | (SEQ ID NO.397) |
| (Ser⁸,Aib³⁵,A6c^{16,20})hGLP-1(7-36)NH₂; | (SEQ ID NO.398) |
| (Ser⁸,Aib³⁵,A6c^{16,29,32})hGLP-1(7-36)NH₂; | (SEQ ID NO.399) |
| (Ser⁸,Aib³⁵,A6c^{20,32})hGLP-1(7-36)NH₂; | (SEQ ID NO.400) |
| (Ser⁸,Aib³⁵,A6c²⁰)hGLP-1(7-36)NH₂; | (SEQ ID NO.401) |
| (Ser⁸,Aib³⁵,Lys²⁵)hGLP-1(7-36)NH₂; | (SEQ ID NO.402) |
| (Ser⁸,Aib^{24,35},A6c²⁰)hGLP-1(7-36)NH₂; | (SEQ ID NO.403) |
| (Ser⁸,Aib³⁵,A6c^{29,32})hGLP-1(7-36)NH₂; | (SEQ ID NO.404) |
| (Ser⁸,Aib^{24,35},A6c^{29,32})hGLP-1(7-36)NH₂: | (SEQ ID NO.405) |
| (Ser⁸,Aib³⁵,A6c¹²)hGLP-1(7-36)NH₂; | (SEQ ID NO.406) |
| (Ser⁸,Aib³⁵,Cha²⁰)hGLP-1(7-36)NH₂; | (SEQ ID NO.407) |
| (Ser⁸,Aib³⁵,A6c³³)hGLP-1(7-36)NH₂; | (SEQ ID NO.408) |
| (Ser⁸,A6c^{16,20},β-Ala³⁵)hGLP-1(7-36)NH₂; | (SEQ ID NO.409) |
| (Ser⁸,Aib³⁵,β-Ala²²)hGLP-1(7-36)NH₂; | (SEQ ID NO.410) |
| (Ser⁸,Aib^{22,35})hGLP-1(7-36)NH₂; | (SEQ ID NO.411) |
| (Ser⁸,Aib³⁵,Glu²³,A6c³²)hGLP-1(7-36)NH₂; | (SEQ ID NO.412) |
| (Ser⁸,Aib^{24,35},Glu²³,A6c³²)hGLP-1(7-36)NH₂; | (SEQ ID NO.413) |
| (Ser⁸,Aib^{24,25,35},Glu²³,A6c³²)hGLP-1(7-36)NH₂; | (SEQ ID NO.414) |
| (Ser⁸,Aib^{24,25,35},A6c^{16,20,32},Glu²³)hGLP-1(7-36)NH₂; | (SEQ ID NO.415) |
| (Ser⁸,A6c³²,β-Ala³⁵)hGLP-1(7-36)NH₂; | (SEQ ID NO.416) |
| (Ser⁸,A5C32,β-Ala³⁵)hGLP-1(7-36)NH₂; | (SEQ ID NO.417) |
| (Ser⁸,Glu²³,β-Ala³⁵)hGLP-1(7-36)NH₂; | (SEQ ID NO.418) |
| (Ser⁸,Aib²⁴,β-Ala³⁵)hGLP-1(7-36)NH₂; | (SEQ ID NO.419) |
| (Ser⁸,Aib³⁰,β-Ala³⁵)hGLP-1(7-36)NH₂; | (SEQ ID NO.420) |
| (Ser⁸,Aib²⁵,β-Ala³⁵)hGLP-1(7-36)NH₂; | (SEQ ID NO.421) |
| (Ser⁸,A6c^{16,29,32},β-Ala³⁵)hGLP-1(7-36)NH₂; | (SEQ ID NO.422) |
| (Ser⁸,A6c^{20,32},β-Ala³⁵)hGLP-1(7-36)NH₂; | (SEQ ID NO.423) |
| (Ser⁸,A6c²⁰,β-Ala³⁵)hGLP-1(7-36)NH₂; | (SEQ ID NO.424) |
| (Ser⁸,Lys²⁵,β-Ala³⁵)hGLP-1(7-36)NH₂; | (SEQ ID NO.425) |
| (Ser⁸,Aib²⁴,A6c²⁰,β-Ala³⁵)hGLP-1(7-36)NH₂; | (SEQ ID NO.426) |
| (Ser⁸,A6c^{29,32},β-Ala³⁵)hGLP-1(7-36)NH₂; | (SEQ ID NO.427) |
| (Ser⁸,Aib²⁴,A6c^{29,32},β-Ala³⁵)hGLP-1(7-36)NH₂; | (SEQ ID NO.428) |
| (Ser⁸,A6c¹²,β-Ala³⁵)hGLP-1(7-36)NH₂; | (SEQ ID NO.429) |
| (Ser⁸,Cha²⁰,β-Ala³⁵)hGLP-1(7-36)NH₂; | (SEQ ID NO.430) |
| (Ser⁸,A6c³³,β-Ala³⁵)hGLP-1(7-36)NH₂; | (SEQ ID NO.431) |
| (Ser⁸,β-Ala^{22,35})hGLP-1(7-36)NH₂; | (SEQ ID NO.432) |
| (Ser⁸,Aib²²,(β-Ala³⁵)hGLP-1(7-36)NH₂; | (SEQ ID NO.433) |
| (Ser⁸,Glu²³,A6c³²,β-Ala³⁵)hGLP-1(7-36)NH₂; | (SEQ ID NO.434) |
| (Ser⁸,Aib²⁴,Glu²³,A6c³²,β-Ala³⁵)hGLP-1(7-36)NH₂; | (SEQ ID NO.435) |
| (Ser⁸,Aib²⁴,Glu²³,A6c³²,Lys³⁴(N^{e}octanoyl),β- | (SEQ ID NO.436) |
| Ala³⁵)hGLP-1(7-36)NH₂; | |
| (Ser⁸,Aib^{24,25},Glu²³,A6c³²,β-Ala³⁵)hGLP-1(7-36)NH₂; | (SEQ ID NO.437) |
| (Ser⁸,Aib^{24,25},A6c^{16,20,32},Glu²³,(β-Ala³⁵)hGLP-1(7-36)NH₂; | (SEQ ID NO.438) |
| (Ser⁸,Aib³⁵,D-Arg³⁶)hGLP-1(7-36)NH₂; | (SEQ ID NO.439) |
| (Ser⁸,Aib³⁵,D-Lys³⁵)hGLP-1(7-36)NH₂; | (SEQ ID NO.440) |
| (Ser⁸,β-Ala³⁵,D-Arg³⁶)hGLP-1(7-36)NH₂; | (SEQ ID NO.441) |
| (Ser⁸,β-Ala³⁵,D-Lys³⁶)hGLP-1(7-36)NH₂; | (SEQ ID NO.442) |
| (Ser⁸,Aib³⁵,Arg^{26,34})hGLP-1(7-36)NH₂; | (SEQ ID NO.443) |
| (Ser⁸,Arg^{26,34},β-Ala³⁵)hGLP-1(7-36)NH₂; | (SEQ ID NO.444) |
| (Ser⁸,Aib³⁵,Arg^{25,26,34})hGLP-1(7-36)NH₂; | (SEQ ID NO.445) |
| (Ser⁸,Arg^{25,26,34},β-Ala³⁵)hGLP-1(7-36)NH₂; | (SEQ ID NO.446) |
| (Ser⁸,Arg^{26,34},β-Ala³⁵,Lys³⁵(N^{ε}-tetradecanoyl))hGLP-1(7-36)OH; | (SEQ ID NO.447) |
| (Ser⁸,Aib³⁵,Arg^{26,34},Lys³⁶(N^{ε}-tetradecanoyl))hGLP-1(7-37)OH; | (SEQ ID NO.448) |
| (Ser⁸,Aib^{35,37},Arg^{26,34},Lys³⁶(N^{ε}-tetradecanoyl))hGLP-1(7-37)OH; | (SEQ ID NO.449) |
| (Ser⁸,Aib³⁵,Arg^{26,34},Lys³⁶(N^{ε}-tetradecanoyl),D | (SEQ ID NO.450) |
| Ala³⁷)hGLP-1(7-37)OH; | |
| (Ser⁸,Aib^{35,37},Arg^{26,34},Lys³⁸(N^{ε}-tetradecanoyl))hGLP-1(7-38)OH; | (SEQ ID NO.451) |
| (Ser⁸,Aib³⁵,Arg^{26,34},β-Ala³⁷,Lys³⁸(N^{ε}- | (SEQ ID NO.452) |
| tetradecanoyl))hGLP-1(7-38)OH; | |
| (Ser⁸,Aib³⁵,Arg^{26,34},Lys³⁸(N^{ε}-tetradecanoyl))hGLP-1(7-38)OH; | (SEQ ID NO.453) |
| (Ser⁸,Arg^{26,34},Lys³⁶(N^{ε}-tetradecanoyl),β-Ala³⁷)hGLP-1(7-37)OH; | (SEQ ID NO.454) |
| (Ser⁸,Aib³⁷,Arg^{26,34},Lys³⁶(N^{ε}-tetradecanoyl))hGLP-1(7-37)OH; | (SEQ ID NO.455) |
| (Ser⁸,Aib³⁵,Arg^{26,34},Ado³⁷)hGLP-1(7-37)OH; | (SEQ ID NO.456) |
| (Ser⁸,Aib³⁵,Arg^{26,34},Ado³⁷)hGLP-1(7-37)NH₂; | (SEQ ID NO.457) |
| (Ser⁸,Arg^{26,34},Lys³⁶(N^{ε}-tetradecanoyl),D-Ala³⁷)hGLP-1(7-37)OH; | (SEQ ID NO.458) |
| (Ser⁸,Aib³⁷,Arg^{26,34},Lys³⁸(N^{ε}-tetradecanoyl))hGLP-1(7-38)OH; | (SEQ ID NO.459) |
| (Ser⁸,Arg^{26,34},β-Ala³⁷,Lys³⁸(N^{ε}-tetradecanoyl))hGLP-1(7-38)OH; | (SEQ ID NO.460) |
| (Ser⁸,Aib³⁵,Lys²⁶(N^{ε}-octanoyl))hGLP-1(7-36)NH₂; | (SEQ ID NO.461) |
| (Ser⁸,Aib³⁵,Lys²⁶(N^{ε}-tetradecanoyl))hGLP-1(7-36)NH₂; | (SEQ ID NO.462) |
| (Ser⁸,Aib³⁵,Lys²⁶(N^{ε}-hexadecanoyI))hGLP-1(7-36)NH₂; | (SEQ ID NO.463) |
| (Ser⁸,Lys²⁶(N^{ε}-octanoyl),β-Ala³⁵)hGLP-1(7-36)NH₂; | (SEQ ID NO.464) |
| (Ser⁸,Lys²⁶(N^{ε}-tetradecanoyl),β-Ala³⁵)hGLP-1(7-36)NH₂; | (SEQ ID NO.465) |
| (Ser⁸,Lys²⁶(N^{ε}-hexadecanoyl),β-Ala³⁵)hGLP-1(7-36)NH₂; | (SEQ ID NO.466) |
| (Ser⁸,Aib³⁵,Lys²⁶(N^{ε}-octanoyl),Arg³⁴)hGLP-1(7-36)NH₂; | (SEQ ID NO.467) |
| (Ser⁸,Aib³⁵,Lys²⁶(N^{ε}-tetradecanoyl),Arg³⁴)hGLP-1(7-36)NH₂; | (SEQ ID NO.468) |
| (Ser⁸,Aib³⁵,Lys²⁶(N^{ε}-hexadecanoyl),Arg³⁴)hGLP-1(7-36)NH₂; | (SEQ ID NO.469) |
| (Ser⁸,Aib³⁵,Lys²⁶(N^{ε}-decanoyl),Arg³⁴)hGLP-1(7-36)NH₂; | (SEQ ID NO.470) |
| (Ser⁸,Aib³⁵,Lys²⁵,Lys²⁶(N^{ε}-octanoyl),Arg³⁴)hGLP-1(7-36)NH₂; | (SEQ ID NO.471) |
| (Ser⁸,Aib³⁵,Lys²⁵,Lys²⁶(N^{ε}-tetradecanoyl), | (SEQ ID NO.472) |
| Arg³⁴)hGLP-1(7-36)NH₂; | |
| (Ser⁸,Aib³⁵,Lys²⁵,Lys²⁶(N^{ε}-hexadecanoyl), | (SEQ ID NO.473) |
| Arg³⁴)hGLP-1(7-36)NH₂; | |
| (Ser⁸,Aib³⁵,Arg^{25,34},Lys²⁶(N^{ε}-octanoyl))hGLP-1(7-36)NH₂; | (SEQ ID NO.474) |
| (Ser⁸,Aib³⁵,Arg^{25,34},Lys²⁶(N^{ε}-tetradecanoyl))hGLP-1(7-36)NH₂; | (SEQ ID NO.475) |
| (Ser⁸,Aib³⁵,Arg^{25,34},Lys²⁶(N^{ε}-hexadecanoyl))hGLP-1(7-36)NH₂; | (SEQ ID NO.476) |
| (Ser⁸,Aib³⁵,Arg^{25,34},Lys²⁶(N^{ε}-decanoyl))hGLP-1(7-36)NH₂; | (SEQ ID NO.477) |
| (Ser⁸,Lys²⁶(N^{ε}-octanoyl),Arg³⁴,β-Ala³⁵)hGLP-1(7-36)NH₂; | (SEQ ID NO.478) |
| (Ser⁸,Lys²⁶(N^{ε}-tetradecanoyl),Arg³⁴,β-Ala³⁵)hGLP-1(7-36)NH₂; | (SEQ ID NO.479) |
| (Ser⁸,Lys²⁶(N^{ε}-hexadecanoyl),Arg³⁴,β-Ala³⁵)hGLP-1(7-36)NH₂; | (SEQ ID NO.480) |
| (Ser⁸,Lys²⁶(N^{ε}-decanoyl),Arg³⁴,β-Ala³⁵)hGLP-1(7-36)NH₂; | (SEQ ID NO.481) |
| (Ser⁸,Aib³⁵,Lys³⁴(N^{ε}-octanoyl))hGLP-1(7-36)NH₂; | (SEQ ID NO.482) |
| (Ser⁸,Aib³⁵,Lys³⁴(N^{ε}-tetradecanoyl))hGLP-1(7-36)NH₂; | (SEQ ID NO.483) |
| (Ser⁸,Aib³⁵,Lys³⁴(N^{ε}-hexadecanoyl))hGLP-1(7-36)NH₂; | (SEQ ID NO.484) |
| (Ser⁸,Aib³⁵,Arg²⁶,Lys³⁴(N^{ε}-octanoyl))hGLP-1(7-36)NH₂; | (SEQ ID NO.485) |
| (Ser⁸,Aib³⁵,Arg²⁶,Lys³⁴(N^{ε}-hexadecanoyl))hGLP-1(7-36)NH₂; | (SEQ ID NO.486) |
| (Ser⁸,Aib³⁵,Arg²⁶,Lys³⁴(N^{ε}-decanoyl))hGLP-1(7-36)NH₂; | (SEQ ID NO.487) |
| (Ser⁸,Aib³⁵,Arg^{25,26},Lys³⁴(N^{ε}-octanoyl))hGLP-1(7-36)NH₂; | (SEQ ID NO.488) |
| (Ser⁸,Aib³⁵,Arg^{25,26},Lys³⁴(N^{ε}-tetradecanoyl))hGLP-1(7-36)NH₂; | (SEQ ID NO.489) |
| (Ser⁸,Aib³⁵,Arg^{25,26},Lys³⁴(N^{ε}-hexadecanoyl))hGLP-1(7-36)NH₂; | (SEQ ID NO.490) |
| (Ser⁸,Aib³⁵,Arg^{25,26},Lys³⁴(N^{ε}-decanoyl))hGLP-1(7-36)NH₂; | (SEQ ID NO.491) |
| (Ser⁸,Aib³⁵,Lys²⁵,Arg²⁶,Lys³⁴(N^{ε}-octanoyl))hGLP-1(7-36)NH₂; | (SEQ ID NO.492) |
| (Ser⁸,Aib³⁵,Lys²⁵,Arg²⁶,Lys³⁴(N^{ε}- | (SEQ ID NO.493) |
| tetradecanoyl))hGLP-1(7-36)NH₂; | |
| (Ser⁸,Aib³⁵,Lys²⁵,Arg²⁶,Lys³⁴(N^{ε}- | (SEQ ID NO.494) |
| hexadecanoyl))hGLP-1(7-36)NH₂; | |
| (Ser⁸,Aib³⁵,Lys³⁶(N^{ε}-octanoyl))hGLP-1(7-36)NH₂; | (SEQ ID NO.495) |
| (Ser⁸,Aib³⁵,Lys³⁶(N^{ε}-tetradecanoyl))hGLP-1(7-36)NH₂; | (SEQ ID NO.496) |
| (Ser⁸,Aib³⁵,Lys³⁶(N^{ε}-hexadecanoyl))hGLP-1(7-36)NH₂; | (SEQ ID NO.497) |
| (Ser⁸,Aib³⁵,Arg²⁶,Lys³⁶(N^{ε}-octanoyl))hGLP-1(7-36)NH₂; | (SEQ ID NO.498) |
| (Ser⁸,Aib³⁵,Arg²⁶,Lys³⁶(N^{ε}-tetradecanoyl))hGLP-1(7-36)NH₂; | (SEQ ID NO.499) |
| (Ser⁸,Aib³⁵,Arg²⁶,Lys³⁶(N^{ε}-hexadecanoyl))hGLP-1(7-36)NH₂; | (SEQ ID NO.500) |
| (Ser⁸,Aib³⁵,Arg^{26,34},Lys³⁶(N^{ε}-octanoyl))hGLP-1(7-36)NH₂; | (SEQ ID NO.501) |
| (Ser⁸,Aib³⁵,Arg^{26,34},Lys³⁶(N^{ε}-hexadecanoyl))hGLP-1(7-36)NH₂; | (SEQ ID NO.502) |
| (Ser⁸,Aib³⁵,Arg^{26,34},Lys³⁸(N^{ε}-octanoyl))hGLP-1(7-38)NH₂; | (SEQ ID NO.503) |
| (Ser⁸,Aib³⁵,Arg^{26,34},Lys³⁸(N^{ε}-decanoyl))hGLP-1(7-38)NH₂; | (SEQ ID NO.504) |
| (Ser⁸,Aib³⁵,Arg^{26,34},Lys³⁸(N^{ε}-tetradecanoyl))hGLP-1(7-38)NH₂; | (SEQ ID NO.505) |
| (Ser⁸,Aib³⁵,Arg^{26,34},Lys³⁸(N^{ε}-hexadecanoyl))hGLP-1(7-38)NH₂; | (SEQ ID NO.506) |
| (Ser⁸,Aib^{35,37},Arg^{25,26,34},Lys³⁸(N^{ε}-octanoyl))hGLP-1(7-38)NH₂; | (SEQ ID NO.507) |
| (Ser⁸,Aib^{35,37},Arg^{25,26,34},Lys³⁸(N^{ε}-decanoyl))hGLP-1(7-38)NH₂; | (SEQ ID NO.508) |
| (Ser⁸,Aib^{35,37},Arg^{25,26,34},Lys³⁸(N^{ε}- | (SEQ ID NO.509) |
| tetradecanoyl))hGLP-1(7-38)NH₂; | |
| (Ser⁸,Aib^{35,37},Arg^{25,26,34},Lys³⁸(N^{ε}- | (SEQ ID NO.510) |
| hexadecanoyl))hGLP-1(7-38)NH₂; | |
| (Ser⁸,Aib^{35,37},Arg^{26,34},Lys³⁸(N^{ε}-octanoyl))hGLP-1(7-38)NH₂; | (SEQ ID NO.511) |
| (Ser⁸,Aib^{35,37},Arg^{26,34},Lys³⁸(N^{ε}-decanoyl))hGLP-1(7-38)NH₂; | (SEQ ID NO.512) |
| (Ser⁸,Aib^{35,37},Arg^{26,34},Lys³⁸(N^{ε}-hexadecanoyl))hGLP-1(7-38)NH₂; | (SEQ ID NO.513) |
| (Ser⁸,Aib³⁵,Lys²⁵,Arg^{26,34},Lys³⁶(N^{ε}-octanoyl))hGLP-1(7-36)NH₂; | (SEQ ID NO.514) |
| (Ser⁸,Aib³⁵,Lys²⁶,Arg^{26,34},Lys³⁶(N^{ε}- | (SEQ ID NO.515) |
| tetradecanoyl))hGLP-1(7-36)NH₂; | |
| (Ser⁸,Aib³⁵,Lys²⁵,Arg^{26,34},Lys³⁶(N^{ε}- | (SEQ ID NO.516) |
| hexadecanoyl))hGLP-1(7-36)NH₂; | |
| (Ser⁸,Aib³⁵,Arg^{25,26,34},Lys³⁶(N^{ε}-octanoyl))hGLP-1(7-36)NH₂; | (SEQ ID NO.517) |
| (Ser⁸,Aib³⁵,Arg^{25,26,34},Lys³⁶(N^{ε}-tetradecanoyl))hGLP-1(7-36)NH₂; | (SEQ ID NO.518) |
| (Ser⁸,Aib³⁵,Arg^{25,26,34},Lys³⁶(N^{ε}-hexadecanoyl))hGLP-1(7-36)NH₂; | (SEQ ID NO.519) |
| (Ser⁸,Aib³⁵,Arg^{25,26,34},Lys³⁶(N^{ε}-decanoyl))hGLP-1(7-36)NH₂; | (SEQ ID NO.520) |
| (Ser⁸,Lys³⁴(N^{ε}-octanoyl),β-Ala³⁵)hGLP-1(7-36)NH₂; | (SEQ ID NO.521) |
| (Ser⁸,Lys³⁴(N^{ε}-tetradecanoyl),β-Ala³⁵)hGLP-1(7-36)NH₂; | (SEQ ID NO.522) |
| (Ser⁸,Lys³⁴(N^{ε}-hexadecanoyt),β-Ala³⁵)hGLP-1(7-36)NH₂; | (SEQ ID NO.523) |
| (Ser⁸,A6c³²,Lys³⁴(N^{e}octanoyl),β-Ala³⁵)hGLP-1(7-36)NH₂; | (SEQ ID NO.524) |
| (Ser⁸,Glu²³,Lys³⁴(N^{e}octanoyl),β-Ala³⁵)hGLP-1(7-36)NH₂; | (SEQ ID NO.525) |
| (Ser⁸,Glu²³,A6c³²,Lys³⁴(N^{e}octanoyl),β-Ala³⁵)hGLP-1(7-36)NH₂; | (SEQ ID NO.526) |
| (Ser⁸,Arg²⁶,Lys³⁴(N^{ε}-octanoyl),β-Ala³⁵)hGLP-1(7-36)NH₂; | (SEQ ID NO.527) |
| (Ser⁸,Arg²⁶,Lys³⁴(N^{ε}-tetradecanoyl),β-Ala³⁵)hGLP-1(7-36)NH₂; | (SEQ ID NO.528) |
| (Ser⁸,Arg²⁶,Lys³⁴(N^{ε}-hexadecanoyl),β-Ala³⁵)hGLP-1(7-36)NH₂; | (SEQ ID NO.529) |
| (Ser⁸,Arg²⁶,Lys³⁴(N^{ε}-decanoyl),β-Ala³⁵)hGLP-1(7-36)NH₂; | (SEQ ID NO.530) |
| (Ser⁸,Arg^{25,26},Lys³⁴(N^{ε}-octanoyl),β-Ala³⁵)hGLP-1(7-36)NH₂; | (SEQ ID NO.531) |
| (Ser⁸,Arg^{25,26},Lys³⁴(N^{ε}-tetradecanoyl),β-Ala³⁵)hGLP-1(7-36)NH₂; | (SEQ ID NO.532) |
| (Ser⁸,Arg^{25,26},Lys³⁴(N^{ε}-hexadecanoyl),β-Ala³⁵)hGLP-1(7-36)NH₂; | (SEQ ID NO.533) |
| (Ser⁸,Arg^{25,26},Lys³⁴(N^{ε}-decanoyl),β-Ala³⁵)hGLP-1(7-36)NH₂; | (SEQ ID NO.534) |
| (Ser⁸,Lys²⁵,Arg²⁶,Lys³⁴(N^{ε}-octanoyl),β-Ala³⁵)hGLP-1(7-36)NH₂; | (SEQ ID NO.535) |
| (Ser⁸,Lys²⁵,Arg²⁶,Lys³⁴(N^{ε}-tetradecanoyl),β- | (SEQ ID NO.536) |
| Ala³⁵)hGLP-1(7-36)NH₂; | |
| (Ser⁸,Lys²⁵,Arg²⁶,Lys³⁴(N^{ε}-hexadecanoyl),β- | (SEQ ID NO.537) |
| Ala³⁵)hGLP-1(7-36)NH₂; | |
| (Ser⁸,β-Ala³⁵,Lys³⁶(N^{ε}-octanoyl))hGLP-1(7-36)NH₂; | (SEQ ID NO.538) |
| (Ser⁸,β-Ala³⁵,Lys³⁶(N^{ε}-tetradecanoyl))hGLP-1(7-36)NH₂; | (SEQ ID NO.539) |
| (Ser⁸,β-Ala³⁵,Lys³⁶(N^{ε}-hexadecanoyl))hGLP-1(7-36)NH₂; | (SEQ ID NO.540) |
| (Ser⁸,Arg²⁶,β-Ala³⁵,Lys³⁶(N^{ε}-octanoyl))hGLP-1(7-36)NH₂; | (SEQ ID NO.541) |
| (Ser⁸,Arg²⁶,β-Ala³⁵,Lys³⁶(N^{ε}-tetradecanoyl))hGLP-1(7-36)NH₂; | (SEQ ID NO.542) |
| (Ser⁸,Arg²⁶,β-Ala³⁵,Lys³⁶(N^{ε}-hexadecanoyl))hGLP-1(7-36)NH₂; | (SEQ ID NO.543) |
| (Ser⁸,Arg^{26,34},β-Ala³⁵,Lys³⁶(N^{ε}-octanoyl))hGLP-1(7-36)NH₂; | (SEQ ID NO.544) |
| (Ser⁸,Arg^{26,34},β-Ala³⁵,Lys³⁶(N^{ε}-tetradecanoyl))hGLP-1(7-36)NH₂; | (SEQ ID NO.545) |
| (Ser⁸,Arg^{26,34},β-Ala³⁵,Lys³⁵(N^{ε}-hexadecanoyl))hGLP-1(7-36)NH₂; | (SEQ ID NO.546) |
| (Ser⁸,Arg^{26,34},β-Ala³⁵,Lys³⁶(N^{ε}-decanoyl))hGLP-1(7-36)NH₂; | (SEQ ID NO.547) |
| (Ser⁸,Lys²⁵,Arg^{26,34},β-Ala³⁵,Lys³⁶(N^{ε}- | (SEQ ID NO.548) |
| octanoyl))hGLP-1(7-36)NH₂; | |
| (Ser⁸,Lys²⁵,Arg^{26,34},Lys³⁶(N^{ε}-tetradecanoyl),β | (SEQ ID NO.549) |
| Ala³⁵)hGLP-1(7-36)NH₂; | |
| (Ser⁸,Lys²⁵,Arg^{26,34},β-Ala³⁵,Lys³⁶(N^{ε}- | (SEQ ID NO.550) |
| hexadecanoyl))hGLP-1(7-36)NH₂; | |
| (Ser⁸,Arg^{25,26,34},β-Ala³⁵,Lys³⁶(N^{ε}-octanoyl))hGLP-1(7-36)NH₂; | (SEQ ID NO.551) |
| (Ser⁸,Arg^{25,26,34},β-Ala³⁵,Lys³⁶(N^{ε}- | (SEQ ID NO.552) |
| tetradecanoyl))hGLP-1(7-36)NH₂; | |
| (Ser⁸,Arg^{25,26,34},β-Ala³⁵,Lys³⁶(N^{ε}- | (SEQ ID NO.553) |
| hexadecanoyl))hGLP-1(7-36)NH₂; | |
| (Ser⁸,Arg^{25,26,34},β-Ala³⁵,Lys³⁶(N^{ε}-decanoyl))hGLP-1(7-36)NH₂; | (SEQ ID NO.554) |
| (Ser⁸,Aib³⁵,Lys²⁶(N^{ε}-octanoyl),A6c³²,Arg³⁴)hGLP-1(7-36)NH₂; | (SEQ ID NO.555) |
| (Ser⁸,Aib³⁵,Lys²⁶(N^{ε}-tetradecanoyl),A6c³², | (SEQ ID NO.556) |
| Arg³⁴)hGLP-1(7-36)NH₂; | |
| (Ser⁸,Aib³⁵,Lys²⁶(N^{ε}-hexadecanoyl),A6c³²,Arg³⁴)hGLP-1(7-36)NH₂; | (SEQ ID NO.557) |
| (Ser⁸,Aib³⁵,A6c³²,Lys³⁴(N^{ε}-octanoyl))hGLP-1(7-36)NH₂; | (SEQ ID NO.558) |
| (Ser⁸,Aib³⁵,A6c³²,Lys³⁴(N^{ε}-tetradecanoyl))hGLP-1(7-36)NH₂; | (SEQ ID NO.559) |
| (Ser⁸,Aib³⁵,A6c³²,Lys³⁴(N^{ε}-hexadecanoyl))hGLP-1(7-36)NH₂; | (SEQ ID NO.560) |
| (Ser⁸,Aib³⁵,Arg²⁶,A6c³²,Lys³⁴(N^{ε}-octanoyl))hGLP-1(7-36)NH₂; | (SEQ ID NO.561) |
| (Ser⁸,Aib³⁵,Arg²⁶,A6c³²,Lys³⁴(N^{ε}- | (SEQ ID NO.562) |
| tetradecanoyl))hGLP-1(7-36)NH₂; | |
| (Ser⁸,Aib³⁵,A6c³²,Lys³⁶(N^{ε}-octanoyl))hGLP-1(7-36)NH₂; | (SEQ ID NO.563) |
| (Ser⁸,Aib³⁵,A6c³²,Lys³⁶(N^{ε}-tetradecanoyl))hGLP-1(7-36)NH₂; | (SEQ ID NO.564) |
| (Ser⁸,Aib³⁵,A6c³²,Lys³⁶(N^{ε}-hexadecanoyl))hGLP-1(7-36)NH₂; | (SEQ ID NO.565) |
| (Ser⁸,Aib³⁵,Arg²⁶,A6c³²,Lys³⁶(N^{ε}-octanoyl))hGLP-1(7-36)NH₂; | (SEQ ID NO.566) |
| (Ser⁸,Aib³⁵,Arg²⁶,A6c³²,Lys³⁶(N^{ε}- | (SEQ ID NO.567) |
| tetradecanoyl))hGLP-1(7-36)NH₂; | |
| (Ser⁸,Aib³⁵,Arg²⁶,A6c³²,Lys³⁶(N^{ε}- | (SEQ ID NO.568) |
| hexadecanoyl))hGLP-1(7-36)NH₂; | |
| (Ser⁸,Aib³⁵,Arg^{26,34},A6c³²,Lys³⁶(N^{ε}-octanoyl))hGLP-1(7-36)NH₂; | (SEQ ID NO.569) |
| (Ser⁸,Aib³⁵,Arg^{26,34},A6c³²,Lys³⁶(N^{ε}-decanoyl))hGLP-1(7-36)NH₂; | (SEQ ID NO.570) |
| (Ser⁸,Aib³⁵,Arg^{26,34},A6c³²,Lys³⁶(N^{ε}- | (SEQ ID NO.571) |
| tetradecanoyl))hGLP-1(7-36)NH₂; | |
| (Ser⁸,Aib³⁵,Arg^{26,34},A6c³²,Lys³⁶(N^{ε}- | (SEQ ID NO.572) |
| hexadecanoyl))hGLP-1(7-36)NH₂; | |
| (Ser⁸,Aib^{24,35},Lys²⁶(N^{ε}-octanoyl),Arg³⁴)hGLP-1(7-36)NH₂; | (SEQ ID NO.573) |
| (Ser⁸,Aib^{24,35},Lys²⁶(N^{ε}-tetradecanoyl),Arg³⁴)hGLP-1(7-36)NH₂; | (SEQ ID NO.574) |
| (Ser⁸,Aib^{24,35},Lys²⁶(N^{ε}-hexadecanoyl),Arg³⁴)hGLP-1(7-36)NH₂; | (SEQ ID NO.575) |
| (Ser⁸,Aib^{24,35},Arg²⁶,Lys³⁴(N^{ε}-octanoyl))hGLP-1(7-36)NH₂; | (SEQ ID NO.576) |
| (Ser⁸,Aib^{24,35},Arg²⁶,Lys³⁴(N^{ε}-tetradecanoyl))hGLP-1(7-36)NH₂; | (SEQ ID NO.577) |
| (Ser⁸,Aib^{24,35},Arg²⁶,Lys³⁴(N^{ε}-hexadecanoyl))hGLP-1(7-36)NH₂; | (SEQ ID NO.578) |
| (Ser⁸,Aib^{24,35},Arg^{26,34},Lys³⁶(N^{ε}-octanoyl))hGLP-1(7-36)NH₂; | (SEQ ID NO.579) |
| (Ser⁸,Aib^{24,35},Arg^{26,34},Lys³⁶(N^{ε}-tetradecanoyl))hGLP-1(7-36)NH₂; | (SEQ ID NO.580) |
| (Ser⁸,Aib^{24,35},Arg^{26,34},Lys³⁶(N^{ε}-hexadecanoyl))hGLP-1(7-36)NH₂; | (SEQ ID NO.581) |
| (Ser⁸,Aib^{24,35},Glu²³,A6c³²,Lys³⁴(N^{e}octanoyl))hGLP-1(7-36)NH₂; | (SEQ ID NO.582) |
| (Ser⁸,Aib³⁵,Glu²³,Lys²⁶(N^{ε}-octanoyl),Arg³⁴)hGLP-1(7-36)NH₂; | (SEQ ID NO.583) |
| (Ser⁸,Aib³⁵,Glu²³,Lys²⁶(N^{ε}-tetradecanoyl), | (SEQ ID NO.584) |
| Arg³⁴)hGLP-1(7-36)NH₂; | |
| (Ser⁸,Aib³⁵,Glu²³,Lys²⁶(N^{ε}-hexadecanoyl), | (SEQ ID NO.585) |
| Arg³⁴)hGLP-1(7-36)NH₂; | |
| (Ser⁸,Aib³⁵,Glu²³,Lys³⁴(N^{e}octanoyl))hGLp-1(7-36)NH₂; | (SEQ ID NO.586) |
| (Ser⁸,Aib³⁵,Glu²³,A6c³²,Lys³⁴(N^{e}octanoyl))hGLP-1(7-36)NH₂; | (SEQ ID NO.587) |
| (Ser⁸,Aib³⁵,Glu²³,Arg²⁶,Lys³⁴(N^{ε}-octanoyl))hGLP-1(7-36)NH₂; | (SEQ ID NO.588) |
| (Ser⁸,Aib³⁵,Glu²³,Arg²⁶,Lys³⁴(N^{ε}- | (SEQ ID NO.589) |
| tetradecanoyl))hGLP-1(7-36)NH₂; | |
| (Ser⁸,Aib³⁵,Glu²³,Arg²⁶,Lys³⁴(N^{ε}- | (SEQ ID NO.590) |
| hexadecanoyl))hGLP-1(7-36)NH₂; | |
| (Ser⁸,Aib³⁵,Glu²³,Lys³⁶(N^{ε}-octanoyl))hGLP-1(7-36)NH₂; | (SEQ ID NO.591) |
| (Ser⁸,Aib³⁵,Glu²³,Lys³⁶(N^{ε}-tetradecanoyl))hGLP-1(7-36)NH₂; | (SEQ ID NO.592) |
| (Ser⁸,Aib³⁵,Glu²³,Lys³⁶(N^{ε}-hexadecanoyl))hGLP-1(7-36)NH₂; | (SEQ ID NO.593) |
| (Ser⁸,Aib³⁵,Glu²³,Arg^{26,34},Lys³⁶(N^{ε}-octanoyl))hGLP-1(7-36)NH₂; | (SEQ ID NO.594) |
| (Ser⁸,Aib³⁵,Glu²³,Arg^{26,34},Lys³⁶(N^{ε}- | (SEQ ID NO.595) |
| tetradecanoyl))hGLP-1(7-36)NH₂; | |
| (Ser⁸,Aib³⁵,Glu²³,Arg^{26,34},Lys³⁶(N^{ε}- | (SEQ ID NO.596) |
| hexadecanoyl))hGLP-1(7-36)NH₂; | |
| (Ser⁸,Aib^{30,35},Lys²⁶(N^{ε}-octanoyl),Arg³⁴)hGLP-1(7-36)NH₂; | (SEQ ID NO.597) |
| (Ser⁸,Aib^{30,35},Lys²⁶(N^{ε}-tetradecanoyl),Arg³⁴)hGLP-1(7-36)NH₂; | (SEQ ID NO.598) |
| (Ser⁸,Aib^{30,35},Lys²⁶(N^{ε}-hexadecanoyl),Arg³⁴)hGLP-1(7-36)NH₂; | (SEQ ID NO.599) |
| (Ser⁸,Aib^{30,35},Arg²⁶,Lys³⁴(N^{ε}-octanoyl))hGLP-1(7-36)NH₂; | (SEQ ID NO.600) |
| (Ser⁸,Aib^{30,35},Arg²⁶,Lys³⁴(N^{ε}-tetradecanoyl))hGLP-1(7-36)NH₂; | (SEQ ID NO.601) |
| (Ser⁸,Aib^{30,35},Arg²⁶,Lys³⁴(N^{ε}-hexadecanoyl))hGLP-1(7-36)NH₂; | (SEQ ID NO.602) |
| (Ser⁸,Aib^{30,35},Arg^{26,34},Lys³⁶(N^{ε}-octanoyl))hGLP-1(7-36)NH₂; | (SEQ ID NO.603) |
| (Ser⁸,Aib^{30,35},Arg^{26,34},Lys³⁶(N^{ε}-tetradecanoyl))hGLP-1(7-36)NH₂; | (SEQ ID NO.604) |
| (Ser⁸,Aib^{30,35},Arg^{26,34},Lys³⁶(N^{ε}-hexadecanoyl))hGLP-1(7-3a)NH₂; | (SEQ ID NO.605) |
| (Ser⁸,Aib³⁵,Glu²³,A6c³²,Lys³⁶(N^{ε}-octanoyl))hGLP-1(7-36)NH₂; | (SEQ ID NO.606) |
| (Ser⁸,Aib³⁵,Glu²³,A6c³²,Lys³⁶(N^{ε}- | (SEQ ID NO.607) |
| tetradecanoyl))hGLP-1(7-36)NH₂; | |
| (Ser⁸,Aib³⁵,Glu²³,A6c³²,Lys³⁶(N^{ε}- | (SEQ ID NO.608) |
| hexadecanoyl)hGLP-1(7-36)NH₂; | |
| (Ser⁸,Aib³⁵,Glu²³,Arg^{26,34},A6c³²,Lys³⁶(N^{ε}- | (SEQ ID NO.609) |
| octanoyl))hGLP-1(7-36)NH₂; | |
| (Ser⁸,Aib³⁵,Glu²³,Arg^{26,34},A6c³²,Lys³⁶(N^{ε}- | (SEQ ID NO.610) |
| tetradecanoyl))hGLP-1(7-36)NH₂; | |
| (Ser⁸,Aib³⁵,Glu²³,Arg^{26,34},A6c³²,Lys³⁶(N^{ε}- | (SEQ ID NO.611) |
| hexadecanoyl))hGLP-1(7-36)NH₂; | |
| (Ser⁸,Aib^{24,35},Glu²³,Arg^{26,34},A6c³²,Lys³⁶(N^{ε}- | (SEQ ID NO.612) |
| octanoyl))hGLP-1(7-36)NH₂; | |
| (Ser⁸,Aib^{24,35},Glu²³,Arg^{26,34},A6c³²,Lys³⁶(N^{ε}- | (SEQ ID NO.613) |
| tetradecanoyl))hGLP-1(7-36)NH₂; | |
| (Ser⁸,Aib^{24,35},Glu²³,Arg^{26,34},A6c³²,Lys³⁶(N^{ε}- | (SEQ ID NO.614) |
| hexadecanoyl))hGLP-1(7-36)NH₂; | |
| (Ser⁸,Aib^{24,30,35},Glu²³,Arg^{26,34},A6c³²,Lys³⁶(N^{ε}- | (SEQ ID NO.615) |
| octanoyl))hGLP-1(7-36)NH₂; | |
| (Ser⁸,Aib^{24,30,35},Glu²³,Arg^{26,34},A6c³²,Lys³⁵(N^{ε}- | (SEQ ID NO.616) |
| tetradecanoyl))hGLP-1(7-36)NH₂; | |
| (Ser⁸,Aib^{24,30,35},Glu²³,Arg^{26,34},A6c³²,Lys³⁶(N^{ε}- | (SEQ ID NO.617) |
| hexadecanoyl))hGLP-1(7-36)NH₂; | |
| ((N^{α}-HEPES-His)⁷,Ser⁸,β-Ala³⁵)hGLP-1(7-36)NH₂; | (SEQ ID NO.618) |
| ((N^{α}-HEPA-His)⁷,Ser⁸,β-Ala³⁵)hGLP-1(7-36)NH₂; | (SEQ ID NO.619) |
| ((N^{α}-tetradecanoyl-His)⁷,Ser⁸,Aib³⁵)hGLP-1(7-36)NH₂; | (SEQ ID NO.620) |
| ((N^{α}-tetradecanoyl-His)⁷,Ser⁸,β-Ala³⁵)hGLP-1(7-36)NH₂; | (SEQ ID NO.621) |
| ((N^{α}-tetradecanoyl-His)⁷,Ser⁸,Aib³⁵,Arg^{26,34})hGLP-1(7-36)NH₂; | (SEQ ID NO.622) |
| ((N^{α}-tetradecanoyl-His)⁷,Ser⁸,Arg^{26,34},β-Ala³⁵)hGLP-1(7-36)NH₂; | (SEQ ID NO.623) |
| ((N^{α}-tetradecanoyl-His)⁷,Ser⁸,Aib³⁵,Arg^{25,26,34})hGLP-1(7-36)NH₂; | (SEQ ID NO.624) |
| ((N^{α}-tetradecanoyl-His)⁷,Ser⁸,Arg^{25,26,34},β- | (SEQ ID NO.625) |
| Ala³⁵)hGLP-1(7-36)NH₂; | |
| (Ser⁸,Aib³⁵,Lys²⁶(N^{ε}-octanesulfonyl),Arg³⁴)hGLP-1(7-36)NH₂; | (SEQ ID NO.626) |
| (Ser⁸,Aib³⁵,Lys²⁶(N^{ε}-dodecanesulfonyl),Arg³⁴)hGLP-1(7-36)NH₂; | (SEQ ID NO.627) |
| (Ser⁸,Aib³⁵,Lys²⁶(N^{ε}-hexadecanesulfonyl), | (SEQ ID NO.628) |
| Arg³⁴)hGLP-1(7-36)NH₂; | |
| (Ser⁸,Aib³⁵,Arg²⁶,Lys³⁴(N^{ε}-octanesulfonyl))hGLP-1(7-36)NH₂; | (SEQ ID NO.629) |
| (Ser⁸,Aib³⁵,Arg²⁶,Lys³⁴(N^{ε}-dodecanesulfonyl))hGLP-1(7-36)NH₂; | (SEQ ID NO.630) |
| (Ser⁸,Aib³⁵,Arg²⁶,Lys³⁴(N^{ε}-hexadecanesulfonyl)) | (SEQ ID NO.631) |
| hGLP-1(7-36)NH₂; | |
| (Ser⁸,Aib³⁵,Arg^{26,34},Lys³⁶(N^{ε}-octanesulfonyl))hGLP-1(7-36)NH₂; | (SEQ ID NO.632) |
| (Ser⁸,Aib³⁵,Arg^{26,34},Lys³⁶(N^{ε}- | (SEQ ID NO.633) |
| hexadecanesulfonyl))hGLP-1(7-36)NH₂; | |
| (Ser⁸,Aib³⁵,Asp²⁶(1-(4-decylpiperazine)),Arg³⁴)hGLP-1(7-36)NH₂; | (SEQ ID NO.634) |
| (Ser⁸,Aib³⁵,Asp²⁶(1-(4-dodecylpiperazine)), | (SEQ ID NO.635) |
| Arg³⁴)hGLP-1(7-36)NH₂; | |
| (Ser⁸,Aib³⁵,Asp²⁶(1 -(4-tetradecylpiperazine)), | (SEQ ID NO.636) |
| Arg³⁴)hGLP-1(7-36)NH₂; | |
| (Ser⁸,Aib³⁵,Asp²⁶(1 -(4-hexadecylpiperazine)), | (SEQ ID NO.637) |
| Arg³⁴)hGLP-1(7-36)NH₂; | |
| (Ser⁸,Aib³⁵,Arg²⁶,Asp³⁴(1-(4-decylpiperazine)))hGLP-1(7-36)NH₂; | (SEQ ID NO.638) |
| (Ser⁸,Aib³⁵,Arg²⁶,Asp³⁴(1-(4- | (SEQ ID NO.639) |
| dodecylpiperazine)))hGLP-1(7-36)NH₂; | |
| (Ser⁸,Aib³⁵,Arg²⁶1,Asp³⁴(1-(4- | (SEQ ID NO.640) |
| tetradecylpiperazine)))hGLP-1(7-36)NH₂; | |
| (Ser⁸,Aib³⁵,Arg²⁶,Asp³⁴(1-(4- | (SEQ ID NO.641) |
| hexadecylpiperazine)))hGLP-1(7-36)NH₂; | |
| (Ser⁸,Aib³⁵,Arg^{26,34},Asp³⁶(1-(4- | (SEQ ID NO.642) |
| decylpiperazine)))hGLP-1(7-36)NH₂; | |
| (Ser⁸,Aib³⁵,Arg^{26,34},Asp³⁶(1-(4- | (SEQ ID NO.643) |
| doclecylpiperazine)))hGLP-1(7-36)NH₂; | |
| (Ser⁸,Aib³⁵,Arg^{26,34},Asp³⁶(1-(4- | (SEQ ID NO.644) |
| hexadecylpiperazine)))hGLP-1(7-36)NH₂; | |
| (Ser⁸,Aib³⁵,Arg^{26,34},Asp³⁸(1-(4- | (SEQ ID NO.645) |
| decylpiperazine)))hGLP-1(7-38)NH₂; | |
| (Ser⁸,Aib³⁵,Arg^{26,34},Asp³⁸(1-(4- | (SEQ ID NO.646) |
| dodecylpiperazine)))hGLP-1(7-38)NH₂; | |
| (Ser⁸,Aib³⁵,Arg^{26,34},Asp³⁸(1-(4- | (SEQ ID NO.647) |
| tetradecylpiperazine)))hGLP-1(7-38)NH₂; | |
| (Ser⁸,Aib³⁵,Arg^{26,34},Asp³⁸(1-(4- | (SEQ ID NO.648) |
| hexadecylpiperazine)))hGLP-1(7-38)NH₂; | |
| (Ser⁸,Aib^{35,37},Arg^{26,34},Asp³⁸(1-(4- | (SEQ ID NO.649) |
| decylpiperazine)))hGLP-1(7-38)NH₂; | |
| (Ser⁸,Aib^{35,37},37,Arg^{26,34},Asp³⁸(1-(4- | (SEQ ID NO.650) |
| dodecylpiperazine)))hGLP-1(7-38)NH₂; | |
| (Ser⁸,Aib^{35,37},Arg^{26,34},Asp³⁸(1-(4- | (SEQ ID NO.651) |
| tetradecylpiperazine)))hGLP-1(7-38)NH₂; | |
| (Ser⁸,Aib^{35,37},Arg^{26,34},Asp³⁸(1-(4- | (SEQ ID NO.652) |
| hexadecylpiperazine)))hGLP-1(7-38)NH₂; | |
| (Ser⁸,Aib³⁵,Arg^{25,34},Asp²⁶(1-(4- | (SEQ ID NO.653) |
| decylpiperazine)))hGLP-1(7-36)NH₂; | |
| (Ser⁸,Aib³⁵,Arg^{25,34},Asp²⁶(1-(4- | (SEQ ID NO.654) |
| dodecylpiperazine)))hGLP-1(7-36)NH₂; | |
| (Ser⁸,Aib³⁵,Arg^{25,34},Asp²⁶(1-(4- | (SEQ ID NO.655) |
| tetradecylpiperazine)))hGLP-1(7-36)NH₂; | |
| (Ser⁸,Aib³⁵,Arg^{25,34},Asp²⁶(1-(4- | (SEQ ID NO.656) |
| hexadecylpiperazine)))hGLP-1(7-36)NH₂; | |
| (Ser⁸,Aib³⁵,Arg^{25,26},Asp³⁴(1-(4- | (SEQ ID NO.657) |
| decylpiperazine)))hGLP-1(7-36)NH₂; | |
| (Ser⁸,Aib³⁵,Arg^{25,26},Asp³⁴(1-(4- | (SEQ ID NO.658) |
| dodecylpiperazine)))hGLP-1(7-36)NH₂; | |
| (Ser⁸,Aib³⁵,Arg^{25,26},Asp³⁴(1-(4- | (SEQ ID NO.659) |
| tetradecylpiperazine)))hGLP-1(7-36)NH₂; | |
| (Ser⁸,Aib³⁵,Arg^{25,26},Asp³⁴(1-(4- | (SEQ ID NO.660) |
| hexadecylpiperazine)))hGLP-1(7-36)NH₂; | |
| (Ser⁸,Aib³⁵,Arg^{25,26,34},Asp³⁶(1-(4- | (SEQ ID NO.661) |
| decylpiperazine)))hGLP-1(7-36)NH₂; | |
| (Ser⁸,Aib³⁵,Arg^{25,26,34},Asp³⁶(1-(4- | (SEQ ID NO.662) |
| dodecylpiperazine)))hGLP-1(7-36)NH₂; | |
| (Ser⁸,Aib³⁵,Arg^{25,26,34},Asp³⁶(1-(4- | (SEQ ID NO.663) |
| tetradecylpiperazine)))hGLP-1(7-36)NH₂; | |
| (Ser⁸,Aib³⁵,Arg^{25,26,34},Asp³⁶(1-(4- | (SEQ ID NO.664) |
| hexadecylpiperazine)))hGLP-1(7-36)NH₂; | |
| (Ser⁸,Aib³⁵,Arg^{25,26,34},Asp³⁸(1-(4- | (SEQ ID NO.665) |
| decylpiperazine)))hGLP-1(7-38)NH₂; | |
| (Ser⁸,Aib³⁵,Arg^{25,26,34},Asp³⁸(1-(4- | (SEQ ID NO.666) |
| dodecylpiperazine)))hGLP-1(7-38)NH₂; | |
| (Ser⁸,Aib³⁵,Arg^{25,26,34},Asp³⁸(1-(4- | (SEQ ID NO.667) |
| tetradecylpiperazine)))hGLP-1(7-38)NH₂; | |
| (Ser⁸,Aib³⁵,Arg^{25,26,34},Asp³⁸(1-(4- | (SEQ ID NO.668) |
| hexadecylpiperazine)))hGLP-1(7-38)NH₂; | |
| (Ser⁸,Aib^{35,37},Arg^{25,26,34},Asp³⁸(1-(4- | (SEQ ID NO.669) |
| decylpiperazine)))hGLP-1(7-38)NH₂; | |
| (Ser⁸,Aib^{35,37},Arg^{25,26,34},Asp³⁸(1-(4- | (SEQ ID NO.670) |
| dodecyipiperazine)))hGLP-1(7-38)NH₂; | |
| (Ser⁸,Aib^{35,37},Arg^{25,26,34},Asp³⁸(1-(4- | (SEQ ID NO.671) |
| tetradecylpiperazine)))hGLP-1(7-38)NH₂; | |
| (Ser⁸,Aib^{35,37},Arg^{25,26,34},Asp³⁸(1-(4- | (SEQ ID NO.672) |
| hexadecylpiperazine)))hGLP-1(7-38)NH₂; | |
| (Ser⁸,Aib³⁵,Arg^{26,34},Glu³⁶(1-dodecylamino))hGLP-1(7-36)NH₂; | (SEQ ID NO.673) |
| (Ser⁸,Aib³⁵,Glu²⁶(1-dodecylamino),Arg³⁴)hGLP-1(7-36)NH₂; | (SEQ ID NO.674) |
| (Ser⁸,Aib³⁵,Arg²⁶,Glu³⁴(1-dodecylamino))hGLP-1(7-36)NH₂; | (SEQ ID NO.675) |
| (Ser⁸,Aib^{35,37},Arg^{26,34},Glu³⁸(1-dodecylamino))hGLP-1(7-38)NH₂; | (SEQ ID NO.676) |
| (Ser⁸,Aib³⁵,Arg³⁴,Lys²⁶(N^{ε}-(2-(4-decyl-1-piperazine)- | (SEQ ID NO.677) |
| acetyl)))hGLP-1(7-36)NH₂; | |
| (Ser⁸,Aib³⁵,Arg³⁴,Lys²⁶(N^{ε}-(2-(4-dodecyl-1-piperazine)- | (SEQ ID NO.678) |
| acetyl)))hGLP-1(7-36)NH₂; | |
| (Ser⁸,Aib³⁵,Arg³⁴,Lys²⁶(N^{ε}-(2-(4-tetradecyl-1-piperazine)- | (SEQ ID NO.679) |
| acetyl)))hGLP-1(7-36)NH₂; | |
| (Ser⁸,Aib³⁵,Arg³⁴,Lys²⁶(N^{ε}-(2-(4-hexadecyl-1-piperazine)- | (SEQ ID NO.680) |
| acetyl)))hGLP-1(7-36)NH₂; | |
| (Ser⁸,Aib³⁵,Arg²⁶,Lys³⁴(N^{ε}-(2-(4-decyl-1-piperazine)- | (SEQ ID NO.681) |
| acetyl)))hGLP-1(7-36)NH₂; | |
| (Ser⁸,Aib³⁵,Arg²⁶,Lys³⁴(N^{ε}-(2-(4-dodecyl-1-piperazine)- | (SEQ ID NO.682) |
| acetyl))hGLP-1(7-36)NH₂; | |
| (Ser⁸,Aib³⁵,Arg²⁶,Lys³⁴(N^{ε}-(2-(4-tetradecyl-1-piperazine)- | (SEQ ID NO.683) |
| acetyl)))hGLP-1(7-36)NH₂; | |
| (Ser⁸,Aib³⁵,Arg²⁶,Lys³⁴(N^{ε}-(2-(4-hexadecyl-1-piperazine) | (SEQ ID NO.684) |
| acetyl)))hGLP-1(7-36)NH₂; | |
| (Ser⁸,Aib³⁵,Arg^{26,34},Lys³⁶(N^{ε}-(2-(4-hexadecyl-1-piperazine)- | (SEQ ID NO.685) |
| acetyl)))hGLP-1(7-36)NH₂; | |
| (Ser⁸,Aib³⁵,Arg^{26,34},Lys³⁶(N^{ε}-(2-(4-dodecyl-1-piperazine)- | (SEQ ID NO.686) |
| acetyl)))hGLP-1(7-36)NH₂; | |
| (Ser⁸,Aib³⁵,Arg^{26,34},Lys³⁶(N^{ε}-(2-(4-hexadecyl-1-piperazine)- | (SEQ ID NO.687) |
| acetyl)))hGLP-1(7-36)NH₂; | |
| (Ser⁸,Aib³⁵,Arg^{26,34},Lys³⁸(N^{ε}-(2-(4-decyl-1-piperazine)- | (SEQ ID NO.688) |
| acetyl)))hGLP-1(7-38)NH₂; | |
| (Ser⁸,Aib³⁵,Arg^{26,34},Lys³⁸(N^{ε}-(2-(4-dodecyl-1-piperazine) | (SEQ ID NO.689) |
| acetyl)))hGLP-1(7-38)NH₂; | |
| (Ser⁸,Aib³⁵,Arg^{26,34},Lys³⁸(N^{ε}-(2-(4-tetradecyl-1-piperazine)- | (SEQ ID NO.690) |
| acetyl)))hGLP-1(7-38)NH₂; | |
| (Ser⁸,Aib³⁵,Arg^{26,34},Lys³⁸(N^{ε}-(2-(4-hexadecyl-1-piperazine)- | (SEQ ID NO.691) |
| acetyl)))hGLP-1(7-38)NH₂; | |
| (Ser⁸,Aib^{35,37},Arg^{26,34},Lys³⁸(N^{ε}-(2-(4-decyl-1-piperazine) | (SEQ ID NO.692) |
| acetyl)))hGLP-1(7-38)NH₂; | |
| (Ser⁸,Aib^{35,37},Arg^{26,34},Lys³⁸(N^{ε}-(2-(4-dodecyl-1-piperazine)- | (SEQ ID NO.693) |
| acetyl)))hGLP-1(7-38)NH₂; | |
| (Ser⁸,Aib^{35,37},Arg^{26,34},Lys³⁸(N^{ε}-(2-(4-tetradecyl-1-piperazine)- | (SEQ ID NO.694) |
| acetyl)))hGLP-1(7-38)NH₂; | |
| (Ser⁸,Aib^{35,37},Arg^{26,34},Lys³⁸(N^{ε}-(2-(4-hexadecyl-1-piperazine) | (SEQ ID NO.695) |
| acetyl)))hGLP-1(7-38)NH₂; | |
| (Ser⁸,Aib³⁵,Arg^{25,34},Lys²⁶(N^{ε}-(2-(4-decyl-1-piperazine)- | (SEQ ID NO.696) |
| acetyl)))hGLP-1(7-36)NH₂; | |
| (Ser⁸,Aib³⁵,Arg^{25,34},Lys²⁶(N^{ε}-(2-(4-dodecyl-1-piperazine)- | (SEQ ID NO.697) |
| acetyl)))hGLP-1(7-36)NH₂; | |
| (Ser⁸,Aib³⁵,Arg^{25,34},Lys²⁶(N^{ε}-(2-(4-tetradecyl-1-piperazine) | (SEQ ID NO.698) |
| acetyl)))hGLP-1(7-36)NH₂; | |
| (Ser⁸,Aib³⁵,Arg^{25,34},Lys²⁶(N^{ε}-(2-(4-hexadecyl-1-piperazine) | (SEQ ID NO.699) |
| acetyl)))hGLP-1(7-36)NH₂; | |
| (Ser⁸,Aib³⁵,Arg^{25,26},Lys³⁴(N^{ε}-(2-(4-decyl-1-piperazine)- | (SEQ ID NO.700) |
| acetyl)))hGLP-1(7-36)NH₂; | |
| (Ser⁸,Aib³⁵,Arg^{25,26},Lys³⁴(N^{ε}-(2-(4-dodecyl-1-piperazine)- | (SEQ ID NO.701) |
| acetyl)))hGLP-1(7-36)NH₂; | |
| (Ser⁸,Aib³⁵,Arg^{25,26},Lys³⁴(N^{ε}-(2-(4-tetradecyl-1-piperazine) | (SEQ ID NO.702) |
| acetyl)))hGLP-1(7-36)NH₂; | |
| (Ser⁸,Aib³⁵,Arg^{25,26},Lys³⁴(N^{ε}-(2-(4-hexadecyl-1-piperazine) | (SEQ ID NO.703) |
| acetyl)))hGLP-1(7-36)NH₂; | |
| (Ser⁸,Aib³⁵,Arg^{25,26,34},Lys³⁶(N^{ε}-(2-(4-decyl-1-piperazine) | (SEQ ID NO.704) |
| acetyl)))hGLP-1(7-36)NH₂; | |
| (Ser⁸,Aib³⁵,Arg^{25,26,34},Lys³⁶(N^{ε}-(2-(4-dodecyl-1-piperazine) | (SEQ ID NO.705) |
| acetyt)))hGLP-1(7-36)NH₂; | |
| (Ser⁸,Aib³⁵,Arg^{25,26,34},Lys³⁶(N^{ε}-(2-(4-tetradecyl-1-piperazine) | (SEQ ID NO.706) |
| acetyl)))hGLP-1(7-36)NH₂; | |
| (Ser⁸,Aib³⁵,Arg^{25,26,34},Lys³⁶(N^{ε}-(2-(4-hexadecyl-1-piperazine) | (SEQ ID NO.707) |
| acetyl)))hGLP-1(7-36)NH₂; | |
| (Ser⁸,Aib³⁵,Arg^{25,26,34},Lys³⁸(N^{ε}-(2-(4-decyl-1-piperazine)- | (SEQ ID NO.708) |
| acetyl)))hGLP-1(7-38)NH₂; | |
| (Ser⁸,Aib³⁵,Arg^{25,26,34},Lys³⁸(N^{ε}-(2-(4-dodecyl-1-piperazine)- | (SEQ ID NO.709) |
| acetyl)))hGLP-1(7-38)NH₂; | |
| (Ser⁸,Aib³⁵,Arg^{25,26,34},Lys³⁸(N^{ε}-(2-(4-tetradecyl-1-piperazine) | (SEQ ID NO.710) |
| acetyl)))hGLP-1(7-38)NH₂; | |
| (Ser⁸,Aib³⁵,Arg^{25,26,34},Lys³⁹(N^{ε}-(2-(4-hexadecyl-1-piperazine) | (SEQ ID NO.711) |
| acetyl)))hGLP-1(7-38)NH₂; | |
| (Ser⁸,Aib^{35,37},Arg^{25,26,34},Lys³⁸(N^{ε}-(2-(4-decyl-1-piperazine) | (SEQ ID NO.712) |
| acetyl)))hGLP-1(7-38)NH₂; | |
| (Ser⁸,Aib^{35,37},Arg^{25,26,34},Lys³⁸(N^{ε}-(2-(4-dodecyl-1-piperazine) | (SEQ ID NO.713) |
| acetyl)))hGLP-1(7-38)NH₂; | |
| (Ser⁸,Aib^{35,37},Arg^{25,26,34},Lys³⁸(N^{ε}-(2-(4-tetradecyl-1-piperazine)- | (SEQ ID NO.714) |
| acetyl)))hGLP-1(7-38)NH₂; | |
| (Ser⁸,Aib^{35,37},Arg^{25,26,34},Lys³⁸(N^{ε}-(2-(4-hexadecyl-1-piperazine) | (SEQ ID NO.715) |
| acetyl)))hGLP-1(7-38)NH₂; | |
| (Ser⁸,Aib³⁵,Arg^{26,34},Lys³⁶(N^{ε}-decanoyl))hGLP-1(7-36)OH; | (SEQ ID NO.716) |
| (Ser⁸,Aib³⁵,Lys²⁵,Arg^{26,34},Lys³⁶(N^{ε}-decanoyl))hGLP-1(7-36)OH; | (SEQ ID NO.717) |
| (Ser⁸,Aib³⁵,Arg^{26,34},Ava³⁷,Ado³⁸)hGLP-1(7-38)NH₂; | (SEQ ID NO.718) |
| (Ser⁸,Aib³⁵,Arg^{26,34},Asp³⁷,Ava³⁶,Ado³⁹)hGLP-1(7-39)NH₂; | (SEQ ID NO.719) |
| (Ser⁸,Aib³⁵,Arg^{26,34},Aun³⁷)hGLP-1(7-37)NH₂; | (SEQ ID NO.720) |
| (Ser⁸,Aib^{17,35})hGLP-1(7-36)NH₂; | (SEQ ID NO.721) |
| (Ser⁸,Arg^{26,34},β-Ala³⁵,D-Asp³⁷,Ava³⁸,Aun³⁹)hGLP-1(7-39)NH₂; | (SEQ ID NO.722) |
| (Ser⁸,Glu^{22,23},β-Ala³⁵)hGLP-1(7-36)NH₂; | (SEQ ID NO.723) |
| (Ser⁸,Lys¹⁸,β-Ala³⁵)hGLP-1(7-36)NH₂; | (SEQ ID NO.724) |
| (Ser⁸,Leu²⁷,β-Ala³⁵)hGLP-1(7-36)NH₂; | (SEQ ID NO.725) |
| (Ser⁸,Lys³³,β-Ala³⁵)hGLP-1(7-36)NH₂; | (SEQ ID NO.726) |
| (Ser⁸,Lys¹⁸,Leu²⁷,(β-Ala³⁵)hGLP-1(7-36)NH₂; | (SEQ ID NO.727) |
| (Ser⁸,D-Arg³⁶)hGLP-1(7-36)NH₂; | (SEQ ID NO.728) |
| (Ser⁸,β-Ala³⁵,D-Arg³⁷)hGLP-1(7-37)NH₂; | (SEQ ID NO.729) |
| (Ser⁸,Aib²⁷,β-Ala³⁵)hGLP-1(7-36)NH₂; | (SEQ ID NO.730) |
| (Ser⁸,Aib²⁷,β-Ala^{35,37},Arg³⁸)hGLP-1(7-38)NH₂; | (SEQ ID NO.731) |
| (Ser⁸,Aib²⁷,(β-Ala^{35,37},Arg^{38,39})hGLP-1(7-39)NH₂; | (SEQ ID NO.732) |
| (Ser⁸,Lys^{18,27},β-Ala³⁵)hGLP-1(7-36)NH₂; | (SEQ ID NO.733) |
| (Ser⁸,Lys²⁷,β-Ala³⁵)hGLP-1(7-36)NH₂; | (SEQ ID NO.734) |
| (Ser⁸,β-Ala³⁵,Arg³⁸)hGLP-1(7-38)NH₂; | (SEQ ID NO.735) |
| (Ser⁸,Arg^{26,34},β-Ala³⁵,)hGLP-1(7-36)NH₂; | (SEQ ID NO.736) |
| (Ser⁸,D-Arg³⁵)hGLP-1(7-36)NH₂; | (SEQ ID NO.737) |
| (Ser⁸,β-Ala³⁵,Arg³⁷)hGLP-1(7-37)NH₂; | (SEQ ID NO.738) |
| (Ser⁸,Phe³¹,β-Ala³⁵)hGLP-1(7-36)NH₂; | (SEQ ID NO.739) |
| (Ser⁸,Aib³⁵,Phe³¹)hGLP-1(7-36)NH₂; | (SEQ ID NO.740) |
| (Ser⁸,Aib³⁵,2-Nal³¹)hGLP-1(7-36)NH₂; | (SEQ ID NO.741) |
| (Ser⁸,Aib³⁵,2-Nal^{28,31})hGLP-1(7-36)NH₂; | (SEQ ID NO.742) |
| (Ser⁸,Aib³⁵,Arg^{26,34},2-Nal³¹)hGLP-1(7-36)NH₂; | (SEQ ID NO.743) |
| (Ser⁸,Aib³⁵,Arg^{26,34},Phe³¹)hGLP-1(7-36)NH₂; | (SEQ ID NO.744) |
| (Ser⁸,Aib³⁵,2-Nal^{19,31})hGLP-1(7-36)NH₂; | (SEQ ID NO.745) |
| (Ser⁸,Aib³⁵,2-Nal^{12,31})hGLP-1(7-36)NH₂; | (SEQ ID NO.746) |
| (Ser⁸,Aib³⁵,Lys³⁶(N^{ε}-decanoyl))hGLP-1(7-36)NH₂; | (SEQ ID NO.747) |
| (Ser⁸,Aib³⁵,Arg³⁴,Lys²⁶(N^{ε}-decanoyl))hGLP-1(7-36)NH₂; | (SEQ ID NO.748) |
| (Ser⁸,Aib³⁵,Arg^{26,34},Lys³⁶(N^{ε}-dodecanoyl))hGLP-1(7-36)NH₂; | (SEQ ID NO.749) |
| (Ser⁸,β-Ala³⁵,Ser³⁷(O-decanoyl))hGLP1(7-37)-NH₂; | (SEQ ID NO.750) |
| (Ser⁸,Aib²⁷,β-Ala^{35,37},Arg³⁸,Lys³⁹(N^{ε}-octanoyl))hGLP-1(7-39)NH₂; | (SEQ ID NO.751) |
| (Ser⁸,Arg^{26,34},β-Ala³⁵,Lys³⁷(N^{ε}-octanoyl))hGLP-1(7-37)NH₂; | (SEQ ID NO.752) |
| (Ser⁸,Arg^{26,34},β-Ala³⁵,Lys³⁷(N^{ε}-decanoyl))hGLP-1(7-37)NH₂; | (SEQ ID NO.753) |
| (Ser⁸,Arg^{26,34},β-Ala³⁵,Lys³⁷(N^{ε}-tetradecanoyl))hGLP-1(7-37)NH₂; | (SEQ ID NO.754) |
| (Ser⁸,Arg^{26,34},β-Ala³⁵,Lys³⁷(N^{ε}-dodecanoyl))hGLP-1(7-37)NH₂; | (SEQ ID NO.755) |
| (Ser⁸,Arg^{26,34},β-Ala³⁵,Lys³⁷(N^{ε}-dodecanoyl))hGLP-1(8-37)NH₂; | (SEQ ID NO.756) |
| (Ser⁸,Arg^{26,34},β-Ala³⁵,Lys³⁶(N^{ε}-Aec-decanoyl))hGLP-1(7-36)NH₂; | (SEQ ID NO.757) |
| (Ser⁸,Arg²⁶,Aib³⁵)hGLP-1(7-36)NH₂; | (SEQ ID NO.758) |
| (Ser⁸,Arg²⁶,Phe³¹,Aib³⁵)hGLP-1(7-36)NH₂; | (SEQ ID NO.759) |
| (Ser⁸,Arg²⁶,Phe³¹,β-Ala³⁵)hGLP-1(7-36)NH₂; | (SEQ ID NO.760) |
| (Ser⁸,Arg²⁶,β-Ala³⁵)hGLP-1(7-36)NH₂; | (SEQ ID NO.761) |
| (Ser⁸,Arg^{26,34},Phe³¹,β-Ala³⁵)hGLP-1(7-36)NH₂; | (SEQ ID NO.762) |
| (Ser⁸,Arg³⁴,Aib³⁵)hGLP-1(7-36)NH₂; | (SEQ ID NO.763) |
| (Ser⁸,Arg³⁴,β-Ala³⁵)hGLP-1(7-36)NH₂; | (SEQ ID NO.764) |
| (Ser⁸,Phe³¹,Arg³⁴,Aib³⁵)hGLP-1(7-36)NH₂; | (SEQ ID NO.765) |
| or | |
| (Ser⁸,Phe³¹,Arg³⁴,β-Ala³⁵)hGLP-1(7-36)NH₂; | (SEQ ID NO.766) |
| or | |
| a pharmaceutically acceptable salt thereof. | |

A preferred compound of the group of compounds described in paragraphs [068] through [0453] is a compound according to the formula:

| | |
|---|---|
| (Gly⁸,Aib³⁵,A6c³²)hGLP-1(7-36)NH₂; | (SEQ ID NO.8) |
| (Gly⁸,Aib³⁵,Glu²³)hGLP-1(7-36)NH₂; | (SEQ ID NO.10) |
| (Gly⁸,Aib^{24,35})hGLP-1(7-36)NH₂; | (SEQ ID NO.11) |
| (Gly⁸,Aib³⁵,Glu²³,A6c³²)hGLP-1(7-36)NH₂; | (SEQ ID NO.28) |
| (Gly⁸,Glu²³,β-Ala³⁵)hGLP-1(7-36)NH₂; | (SEQ ID NO.34) |
| (Gly⁸,Aib³⁵,Arg^{26,34})hGLP-1(7-36)NH₂; | (SEQ ID NO.59) |
| (Gly⁸,Aib³⁵,Arg^{26,34},Lys³⁶(N^{ε}-octanoyl))hGLP-1(7-36)NH₂; | (SEQ ID NO.117) |
| (Gly⁸,Aib³⁵,Arg^{26,34},Lys³⁶(N^{ε}-decanoyl))hGLP-1(7-36)OH; | (SEQ ID NO.332) |
| (Gly⁸,Aib³⁵,Lys²⁵,Arg^{26,34},Lys³⁶(N^{ε}-decanoyl))hGLP-1(7-36)OH; | (SEQ ID NO.333) |
| (Gly⁸,Arg^{26,34},β-Ala³⁵,Lys³⁶(N^{ε}-Aec-decanoyl))hGLP-1(7-36)NH₂; | (SEQ ID NO.383) |
| (Gly⁸,Aib³⁵,Arg25,34,Ava³⁷,Ado³⁸)hGLP-1(7-38)NH₂; | (SEQ ID NO.334) |
| (Gly⁸,Aib³⁵,Arg^{26,34},Asp³⁷,Ava³⁸,Ado³⁹)hGLP-1(7-39)NH₂; | (SEQ ID NO.335) |
| (Gly⁸,Aib³⁵,Arg^{26,34},Aun³⁷)hGLP-1(7-37)NH₂; | (SEQ ID NO.336) |
| (Gly⁸,Aib^{17,35})hGLP-1(7-36)NH₂; | (SEQ ID NO.337) |
| (Gly⁸,Arg^{26,34},β-Ala³⁵,D-Asp³⁷,Ava³⁸,Aun³⁹)hGLP-1(7-39)NH₂; | (SEQ ID NO.338) |
| (Gly⁸,Glu^{22,23},β-Ala³⁵)hGLP-1(7-36)NH₂; | (SEQ ID NO.339) |
| (Gly⁸.Lys¹⁸,β-Ala³⁵)hGLP-1(7-36)NH₂; | (SEQ ID NO.340) |
| (Gly⁸,Leu²⁷,β-Ala³⁵)hGLP-1(7-36)NH₂; | (SEQ ID NO.341) |
| (Gly⁸,Lys³³,β-Ala³⁵)hGLP-1(7-36)NH₂; | (SEQ ID NO.342) |
| (Gly⁸,Lys¹⁸,Leu²⁷,β-Ala³⁵)hGLP-1(7-36)NH₂; | (SEQ ID NO.343) |
| (Gly⁸,D-Arg³⁵)hGLP-1(7-36)NH₂; | (SEQ ID NO.344) |
| (Gly⁸,β-Ala³⁵,D-Arg³⁷)hGLP-1(7-37)NH₂; | (SEQ ID NO.345) |
| (Gly⁸,Aib²⁷,β-Ala³⁵)hGLP-1(7-36)NH₂; | (SEQ ID NO.346) |
| (Gly⁸,Aib²⁷,β-Ala^{35,37},Arg³⁸)hGLP-1(7-38)NH₂; | (SEQ ID NO.347) |
| (Gly⁸,Aib²⁷,β-Ala^{35,37},Arg^{38,39})hGLP-1(7-39)NH₂; | (SEQ ID NO.348) |
| (Gly⁸,Lys^{18,27},β-Ala³⁵)hGLP-1(7-36)NH₂; | (SEQ ID NO.349) |
| (Gly⁸,Lys²⁷,β-Ala³⁵)hGLP-1(7-36)NH₂; | (SEQ ID NO.350) |
| (Gly⁸,β-Ala³⁵,Arg³⁸)hGLP-1(7-38)NH₂; | (SEQ ID NO.351) |
| (Gly⁸,Arg^{26,34},β-Ala³⁵,)hGLP-1(7-36)NH₂; | (SEQ ID NO.352) |
| (Gly⁸,D-Arg³⁵)hGLP-1(7-36)NH₂; | (SEQ ID NO.353) |
| (Gly⁸,β-Ala³⁵,Arg³⁷)hGLP-1(7-37)NH₂; | (SEQ ID NO.354) |
| (Gly⁸,Phe³¹,β-Ala³⁵)hGLP-1(7-36)NH₂; | (SEQ ID NO.355) |
| (Gly⁸,Aib³⁵,Phe³¹)hGLP-1(7-36)NH₂; | (SEQ ID NO.356) |
| (Gly⁸,Aib³⁵,2-Nal³¹)hGLP-1(7-36)NH₂; | (SEQ ID NO.357) |
| (Gly⁸,Aib³⁵,2-Nal^{28,31})hGLP-1(7-36)NH₂; | (SEQ ID NO.358) |
| (Gly⁸,Aib³⁵,Arg^{26,34},2-Nal³¹)hGLP-1(7-36)NH₂; | (SEQ ID NO.359) |
| (Gly⁸,Aib³⁵,Arg^{26,34},Phe³¹)hGLP-1(7-36)NH₂; | (SEQ ID NO.360) |
| (Gly⁸,Aib³⁵,2-Nal^{19,31})hGLP-1(7-36)NH₂; | (SEQ ID NO.361) |
| (Gly⁸,Aib³⁵,2-Nal^{12,31})hGLP-1(7-36)NH₂; | (SEQ ID NO.362) |
| (Gly⁸,Aib³⁵,Lys³⁶(N^{ε}-decanoyl))hGLP-1(7-36)NH₂; | (SEQ ID NO.99) |
| (Gly⁸,Aib³⁵,Arg³⁴,Lys²⁶(N^{ε}-decanoyl))hGLP-1(7-36)NH₂; | (SEQ ID NO.364) |
| (Gly⁸,Aib³⁵,Arg^{26,34},Lys³⁶(N^{ε}-dodecanoyl))hGLP-1(7-36)NH₂; | (SEQ ID NO.365) |
| (Gly⁸,β-Ala³⁵,Ser³⁷(O-decanoyl))hGLP1(7-37)-NH₂; | (SEQ ID NO.384) |
| (Gly⁸,Aib²⁷,β-Ala^{35,37},Arg³⁸,Lys³⁹(N^{ε}-octanoyl))hGLP-1(7-39)NH₂; | (SEQ ID NO.367) |
| (Gly⁸,Arg^{26,34},β-Ala³⁵,Lys³⁷(N^{ε}-octanoyl))hGLP-1(7-37)NH₂; | (SEQ ID NO.368) |
| (Gly⁸,Arg^{26,34},β-Ala³⁵,Lys³⁷(N^{ε}-decanoyl))hGLP-1(7-37)NH₂; | (SEQ ID NO.369) |
| or | |
| (Gly⁸,Arg^{26,34},β-Ala³⁵,Lys³⁷(N^{ε}-tetradecanoyl))hGLP-1(7-37)NH₂; | (SEQ ID NO.370) |

or a pharmaceutically acceptable salt thereof.

A preferred compound of the group of compounds described in paragraphs [0455] through [0837] is a compound according to the formula:

| | |
|---|---|
| (Ser⁸,Aib³⁵,A6c³²)hGLP-1(7-36)NH₂; | (SEQ ID NO.392) |
| (Ser⁸,Aib³⁵,Glu²³)hGLP-1(7-36)NH₂; | (SEQ ID NO.394) |
| (Ser⁸,Aib^{24,35})hGLP-1(7-36)NH₂; | (SEQ ID NO.395) |
| (Ser⁸,Aib³⁵,Glu²³,A6c³²)hGLP-1(7-36)NH₂; | (SEQ ID NO.412) |
| (Ser⁸,Glu²³,β-Ala³⁵)hGLP-1(7-36)NH₂; | (SEQ ID NO.418) |
| (Ser⁸,Aib³⁵,Arg^{26,34})hGLP-1(7-36)NH₂; | (SEQ ID NO.443) |
| (Ser⁸,Aib³⁵,Arg^{26,34},Lys³⁶(N^{ε}-octanoyl))hGLP-1(7-36)NH₂; | (SEQ ID NO.501) |
| (Ser⁸,Aib³⁵,Arg^{26,34},Lys³⁶(N^{ε}-decanoyl))hGLP-1(7-36)OH: | (SEQ ID NO.716) |
| (Ser⁸,Aib³⁵,Lys²⁵,Arg^{26,34},Lys³⁶(N^{ε}-decanoyl))hGLP-1(7-36)OH; | (SEQ ID NO.717) |
| (Ser⁸,Arg^{26,34},β-Ala³⁵,Lys³⁶(NEAec-decanoyl))hGLP-1(7-36)NH₂; | (SEQ ID NO.757) |
| (Ser⁸,Aib³⁵,Arg^{26,34},Ava³⁷,Ado³⁸)hGLP-1(7-38)NH₂; | (SEQ ID NO.718) |
| (Ser⁸,Aib³⁵,Arg^{26,34},Asp³⁷,Ava³⁸,Ado³⁹)hGLP-1(7-39)NH₂; | (SEQ ID NO.719) |
| (Ser⁸,Aib³⁵,Arg^{26,34},Aun³⁷)hGLP-1(7-37)NH₂; | (SEQ ID NO.720) |
| (Ser⁸,Aib^{17,35})hGLP-1(7-36)NH₂; | (SEQ ID NO.389) |
| (Ser⁸,Arg^{26,34},β-Ala³⁵,D-Asp³⁷,Ava³⁸,Aun³⁹)hGLP-1(7-39)NH₂; | (SEQ ID NO.722) |
| (Ser⁸,Glu^{22,23},β-Ala³⁵)hGLP-1(7-36)NH₂; | (SEQ ID NO.723) |
| (Ser⁸,Lys¹⁸,β-Ala³⁵)hGLP-1(7-36)NH₂; | (SEQ ID NO.724) |
| (Ser⁸,Leu²⁷,β-Ala³⁵)hGLP-1(7-36)NH₂; | (SEQ ID NO.725) |
| (Ser⁸,Lys³³,β-Ala³⁵)hGLP-1(7-36)NH₂; | (SEQ ID NO.726) |
| (Ser⁸,Lys¹⁸,Leu²⁷,β-Ala³⁵)hGLP-1(7-36)NH₂; | (SEQ ID NO.727) |
| (Ser⁸,D-Arg³⁶)hGLP-1(7-36)NH₂; | (SEQ ID NO.728) |
| (Ser⁸,β-Ala³⁵,D-Arg³⁷)hGLP-1(7-37)NH₂; | (SEQ ID NO.729) |
| (Ser⁸,Aib²⁷,β-Ala³⁵)hGLP-1(7-36)NH₂; | (SEQ ID NO.730) |
| (Ser⁸,Aib²⁷,β-Ala^{35,37},Arg³⁸)hGLP-1(7-38)NH₂; | (SEQ ID NO.731) |
| (Ser⁸,Aib²⁷,β-Ala^{35,37},Arg^{38,39})hGLP-1(7-39)NH₂; | (SEQ ID NO.732) |
| (Ser⁸,Lys^{18,27},β-Ala³⁵)hGLP-1(7-36)NH₂; | (SEQ ID NO.733) |
| (Ser⁸,Lys²⁷,β-Ala³⁵)hGLP-1(7-36)NH₂; | (SEQ ID NO.734) |
| (Ser⁸,β-Ala³⁵,Arg³⁸)hGLP-1(7-38)NH₂; | (SEQ ID NO.735) |
| (Ser⁸,Arg^{26,34},β-Ala³⁵)hGLP-1(7-36)NH₂; | (SEQ ID NO.736) |
| (Ser⁸,D-Arg³⁵)hGLP-1(7-36)NH₂; | (SEQ ID NO.737) |
| (Ser⁸,β-Ala³⁵,Arg³⁷)hGLP-1(7-37)NH₂; | (SEQ ID NO.738) |
| (Ser⁸,Phe³¹,β-Ala³⁵)hGLP-1(7-36)NH₂; | (SEQ ID NO.739) |
| (Ser⁸,Aib³⁵,Phe³¹)hGLP-1(7-36)NH₂; | (SEQ ID NO.740) |
| (Ser⁸,Aib³⁵,2-Nal³¹)hGLP-1(7-36)NH₂; | (SEQ ID NO.741) |
| (Ser⁸,Aib³⁵,2-Nal^{28,31})hGLP-1(7-36)NH₂; | (SEQ ID NO.742) |
| (Ser⁸,Aib³⁵,Arg^{26,34},2-Nal1)hGLP-1(7-36)NH₂; | (SEQ ID NO.743) |
| (Ser⁸,Aib³⁵,Arg^{26,34},Phe³¹)hGLP-1(7-36)NH₂; | (SEQ ID NO.744) |
| (Ser⁸,Aib³⁵,2-Nal^{19,31})hGLP-1(7-36)NH₂; | (SEQ ID NO.745) |
| (Ser⁸,Aib³⁵,2-Nal^{12,31})hGLP-1(7-36)NH₂; | (SEQ ID NO.746) |
| (Ser⁸,Aib³⁵,Lys³⁶(N^{ε}-decanoyl))hGLP-1(7-36)NH₂; | (SEQ ID NO.483) |
| (Ser⁸,Aib³⁵,Arg³⁴,Lys²⁶(N^{ε}-decanoyl))hGLP-1(7-36)NH₂; | (SEQ ID NO.748) |
| (Ser⁸,Aib³⁵,Arg^{26,34},Lys³⁶(N^{ε}-dodecanoyl))hGLP-1(7-36)NH₂; | (SEQ ID NO.749) |
| (Ser⁸,β-Ala³⁵,Ser³⁷(O-decanoyl))hGLP1(7-37)-NH₂; | (SEQ ID NO.539) |
| (Ser⁸,Aib²⁷,(β-Ala^{35,37},Arg³⁸,Lys³⁹(N^{ε}- | (SEQ ID NO.751) |
| octanoyl))hGLP-1(7-39)NH₂; | |
| (Ser⁸,Arg^{26,34},β-Ala³⁵,Lys³⁷(N^{ε}-octanoyl))hGLP-1(7-37)NH₂; | (SEQ ID NO.752) |
| (Ser⁸,Arg^{26,34},β-Ala³⁵,Lys³⁷(N^{ε}-decanoyl))hGLP-1(7-37)NH₂; | (SEQ ID NO.753) |
| or | |
| (Ser⁸,Arg^{26,34},β-Ala³⁵,Lys³⁷(N^{ε}-tetradecanoyl)) | (SEQ ID NO.754) |
| hGLP-1(7-37)NH₂; | |

or a pharmaceutically acceptable salt thereof.

In a more preferred embodiment the invention features a compound according to formula (I), wherein said compound is:

| | |
|---|---|
| (Ser⁸,Aib³⁵)hGLP-1(7-36)NH₂; | (SEQ ID NO.767) |
| (Abu⁸,β-Ala³⁵)hGLP-1(7-36)NH₂; | (SEQ ID NO.768) |
| (Val⁸,β-Ala³⁵)hGLP-1(7-36)NH₂; | (SEQ ID NO.769) |
| (β-Ala^{8,35})hGLP-1(7-36)NH₂; | (SEQ ID NO.770) |
| (Abu⁸,Aib³⁵)hGLP-1(7-36)NH₂; | (SEQ ID NO.771) |
| (Val⁸,Aib³⁵)hGLP-1(7-36)NH₂; | (SEQ ID NO.772) |
| or | |
| (β-Ala⁸,Aib³⁵)hGLP-1(7-36)NH₂; | (SEQ ID NO.773) |

or a pharmaceutically acceptable salt thereof.

In a still more preferred embodiment the invention features a compound according to formula (I), wherein said compound is:

| | |
|---|---|
| (Ser⁸,Aib³⁵)hGLP-1(7-36)NH₂; | (SEQ ID NO.767) |
| (Abu⁸,β-Ala³⁵)hGLP-1(7-36)NH₂; | (SEQ ID NO.768) |
| or | |
| (Abu⁸,Aib³⁵)hGLP-1(7-36)NH₂; | (SEQ ID NO.771) |

or a pharmaceutically acceptable salt thereof.

In yet a still more preferred embodiment the invention features a compound according to formula (I), wherein said compound is:

| | |
|---|---|
| (Ser⁸,Aib³⁵)hGLP-1(7-36)NH₂; | (SEQ ID NO.767) |

or a pharmaceutically acceptable salt thereof.

In another aspect of the invention is featured a pharmaceutical composition comprising an effective amount of a compound according to formula (I), or a pharmaceutically acceptable salt thereof, and a pharmaceutically acceptable carrier or diluent.

In another aspect of the invention is featured a method of eliciting an agonist effect from a GLP-1 receptor in a subject in need thereof, said method comprising administering to said subject an effective amount of a compound according to formula (I) or a pharmaceutically acceptable salt thereof.

In another aspect of the invention is featured a method of treating a disease selected from the group consisting of Type I diabetes, Type II diabetes, obesity, glucagonomas, secretory disorders of the airway, metabolic disorder, arthritis, osteoporosis, central nervous system disease, restenosis and neurodegenerative disease, in a subject in need thereof, said method comprising administering to said subject an effective amount of a compound according to formula (I) or a pharmaceutically acceptable salt thereof. Preferably, said disease is Type I diabetes or Type II diabetes.

In a preferred embodiment of each of the aspects detailed in paragraphs [0953], [0955], and [09561 said compound according to formula (I) is a compound described in paragraph [060], [061], [062], [063], [063], [065], [066], [067], or [068], or a pharmaceutically acceptable salt thereof. More preferably said compound according to formula (I) is selected from among the compounds of Formula (I) that are specifically disclosed herein, or a pharmaceutically acceptable salt thereof. More preferably said compound according to formula (I) is selected from among the compounds disclosed in paragraphs [069] through [0452] or in paragraphs [0455] through [0836], or a pharmaceutically acceptable salt thereof. More preferably said compound according to formula (I) is selected from among the compounds disclosed in paragraphs [0839] through [0885] or in paragraphs [0888] through [0934], or a pharmaceutically acceptable salt thereof. Still more preferably said compound according to formula (I) is selected from among the compounds disclosed in paragraphs [0937] through [0943], or a pharmaceutically acceptable salt thereof. Still more preferably said compound according to formula (I) is selected from among the compounds disclosed in paragraphs [0946] through [0948], or a pharmaceutically acceptable salt thereof. In a most preferred embodiment said compound according to formula (I) is (Ser⁸, Aib³⁵)hGLP-1(7-36)NH₂ (SEQ ID NO.767), or a pharmaceutically acceptable salt thereof.

With the exception of the N-terminal amino acid, all abbreviations (e.g., Ala) of amino acids in this disclosure stand for the structure of —NH—CH(R)—CO—, wherein R is the side chain of an amino acid (e.g., CH₃ for Ala). For the N-terminal amino acid, the abbreviation stands for the structure of (R²R³)—N—CH(R)—CO—, wherein R is a side chain of an amino acid and R² and R³ are as defined above, except when A⁷ is Ura, Paa or Pta, in which case R² and R³ are not present since Ura, Paa and Pta are considered here as des-amino amino acids. Certain other abbreviations for amino acids are employed herein with the following meanings:
Abu α-aminobutyric acid;
Ado 12-aminododecanoic acid;
Aec 4-(2-aminoethyl)-1-carboxymethyl-piperazine, represented by the structure: 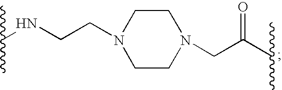
Aib α-aminoisobutyric acid;
Aic 2-aminoindane-2-carboxylic acid;
β-Ala β-alanine;
Amp 4-amino-phenylalanine;
Aun 11-aminoundecanoic acid;
Ava 5-aminovaleric acid;
Cha cyclohexylalanine;
Gaba γ-aminobutyric acid;
hArg homoarginine;
HEPA 4-(2-hydroxyethyl)-1-piperazimyl acetic acid;
HEPES 4-(2-hydroxyethyl)-1-piperazine-ethane sulfonic acid;
N-Me-Ala N-methyl-alanine;
N-Me-Asp N-methyl-aspartic acid;
N-Me-Glu N-methyl-glutamic acid;
N-Me-Gly N-methyl-glycine;
N^{α}-Me-His N^{α}-methyl-histidine;
1-Nal β-(1-naphthyl)alanine;
2-Nal β-(2-naphthyl)alanine;
Nie norleucine;
Orn ornithine;
Paa trans-3-(3-pyridyl) acrylic acid;
3-Pal β-(3-pyridinyl)alanine;
4-Pal β-(4-pyridinyl)alanine;
Pta (4-pyridylthio) acetic acid;
Tle tert-butylglycine;
Tma-His N,N-tetramethylamidino-histidine;
Tba tert-butylalanine; and
Ura urocanic acid.

What is meant by Acc is an amino acid selected from the group of 1-amino-1-cyclopropanecarboxylic acid (A3c); 1-amino-1-cyclobutanecarboxylic acid (A4c); 1-amino-1-cyclopentanecarboxylic acid (A5c); 1-amino-1-cyclohexanecarboxylic acid (A6c); 1-amino-1-cycloheptanecarboxylic acid (A7c); 1-amino-1-cyclooctanecarboxylic acid (A8c); and 1-amino-1-cyclononanecarboxylic acid (A9c).

The terms hydroxyalkyl, hydroxyphenylalkyl, and hydroxynaphthylalkyl each denotes a moiety containing 1, 2, 3, or 4 hydroxy substituents.

The term COX⁵ stands for —C═O.X⁵. Examples of —C═O.X⁵ Include, but are not limited to, acetyl and phenylpropionyl.

What is meant by Lys(N^{ε}-alkanoyl) is represented by the following structure:

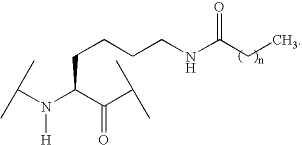

What is meant by Lys(N^{ε}-alkylsufonyl) is represented by the following structure:

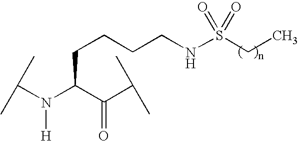

What is meant by Lys(N^{ε}-(2-(4-alkyl-1-piperazine)-acetyl)) is represented by the following structure:

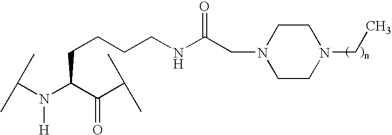

What is meant by Asp(1-(4-alkyl-piperazine)) is represented by the following structure:

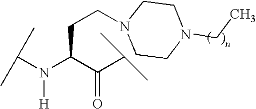

What is meant by Asp(1-alkylamino) is represented by the following structure:

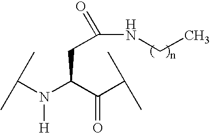

What is meant by Lys(N^{ε}-Aec-alkanoyl) is represented by the structure:

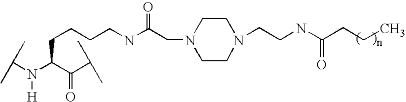

What is meant by Lys(N^{ε}-ace-alkanoyl) is represented by the structure:

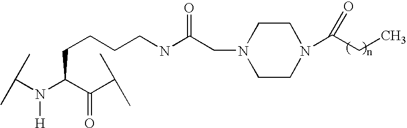

What is meant by Ser(O-alkanoyl) is represented by the structure:

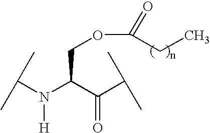

The variable n in the structure depicted for Lys(N^{ε}-alkanoyl), Lys(N^{ε}-alkylsulfonyl), Lys(N^{ε}-(2-(4-alkyl-1-piperazine)-acetyl)), Asp(1-(4-alkyl-piperazine)), Asp(1-alkylamino), Lys(N^{ε}-Aec-alkanoyl), (N^{ε}-ace-alkanoyl) and Ser(O-alkanoyl) is an integer from 1 through 30, inclusive.

The full names for other abbreviations used herein are as follows:
Boc t-butyloxycarbonyl;
2BrZ 2-bromobenzyloxycarbonyl;
Bzl benzyl;
2ClZ 2-chlorobenzyloxycarbonyl;
DCM dichloromethane;
DIEA diisopropylethylamine;
DMF dimethylformamide;
DNP 2,4-dinitrophenyl;
Fm formyl;
Fmoc 9-fluorenylmethoxycarbonyl;
HBTU 2-(1H-Benzotriazol-1-yl)-1,1,3,3-tetramethyl uronium hexafluorophosphate;
HF hydrogen fluoride;
HOAc acetic acid;
HOBt N-hydroxybenzotriazole;
OcHex O-cyclohexyl;
PAM resin 4-hydroxymethylphenylacetamidomethyl resin;
TFA trifluoroacetic acid;
Tos tosyl; and
Xan xanthyl.

The term “(C₁-C₃₀)hydrocarbon moiety” encompasses alkyl, alkenyl and alkynyl, and in the case of alkenyl and alkynyl there are C₂-C₃₀.

The term “halo” encompasses fluoro, chloro, bromo and iodo.

A peptide of this invention is also denoted herein by another format, e.g., (A5c⁸)hGLP-1(7-36)NH₂(SEQ ID NO.774), with the substituted amino acids from the natural sequence placed between the first set of parentheses (e.g., A5c⁸ for Ala⁸ in hGLP-1). The abbreviation GLP-1 means glucagon-like peptide-1; hGLP-1 means human glucagon-like peptide-1. The numbers between the parentheses following “hGLP-1” refer to the number of amino acids present in the peptide. For example, hGLP-1(7-36) (SEQ ID NO.775) denotes amino acids 7 through 36 of the peptide sequence for human GLP-1. The sequence for hGLP-1(7-37) (SEQ ID NO.776) is listed in Mojsov, S., Int. J. Peptide Protein Res,. 40, 1992, pp. 333-342. The designation “NH₂” in hGLP-1(7-36)NH₂ indicates that the C-terminus of the peptide is amidated. hGLP-1(7-36) (SEQ ID NO.775) means that the C-terminus is the free acid. In hGLP-1(7-38) (SEQ ID NO.777), residues in positions 37 and 38 are Gly and Arg, respectively.

### DETAILED DESCRIPTION

The peptides of this invention can be prepared by standard solid phase peptide synthesis. See, e.g., Stewart, J. M., et al., Solid Phase Synthesis (Pierce Chemical Co., 2d ed. 1984). The substituents R² and R³ of the above generic formula may be attached to the free amine of the N-terminal amino acid by standard methods known in the art. For example, alkyl groups, e.g., (C₁-C₃₀)alkyl, may be attached using reductive alkylation. Hydroxyalkyl groups, e.g., (C₁-C₃₀)hydroxyalkyl, may also be attached using reductive alkylation wherein the free hydroxy group is protected with a t-butyl ester. Acyl groups, e.g., COE¹, may be attached by coupling the free acid, e.g., E¹COOH, to the free amine of the N-terminal amino acid by mixing the completed resin with 3 molar equivalents of both the free acid and diisopropylcarbodiimide in methylene chloride for one hour. If the free acid contains a free hydroxy group, e.g., p-hydroxyphenylpropionic acid, then the coupling should be performed with an additional 3 molar equivalents of HOBT.

When R¹ is NH—X²—CH₂—CONH₂, (i.e., Z⁰=CONH₂), the synthesis of the peptide starts with BocHN—X²—CH₂—COOH which is coupled to the MBHA resin. If R¹ is NH—X²—CH₂—COOH, (i.e., Z⁰=COOH) the synthesis of the peptide starts with Boc-HN—X²—CH₂—COOH which is coupled to PAM resin. For this particular step, 4 molar equivalents of Boc-HN—X²—COOH, HBTU and HOBt and 10 molar equivalents of DIEA are used. The coupling time is about 8 hours.

The protected amino acid 1-(N-tert-butoxycarbonyl-amino)-1-cyclohexane-carboxylic acid (Boc-A6c-OH) was synthesized as follows. 19.1 g (0.133 mol) of 1-amino-1-cyclohexanecarboxylic acid (Acros Organics, Fisher Scientific, Pittsburgh, Pa.) was dissolved in 200 ml of dioxane and 100 ml of water. To this was added 67 ml of 2N NaOH and the solution was cooled in an ice-water bath. 32.0 g (0.147 mol) of di-tert-butyl-dicarbonate was added to the solution and the reaction mixture was stirred overnight at room temperature. Dioxane was then removed under reduced pressure and 200 ml of ethyl acetate was added to the remaining aqueous solution. The mixture was cooled in an ice-water bath and the pH of the aqueous layer was adjusted to about 3 by adding 4N HCl. The organic layer was separated and the aqueous layer was extracted with ethyl acetate (1×100 ml). The two organic layers were combined, washed with water (2×150 ml), dried over anhydrous MgSO₄, filtered, and concentrated to dryness under reduced pressure. The residue was recrystallized in ethyl acetate/hexanes to produce 9.2 g (0.0386 mol) of the purified product; i.e., a 29% yield from the starting material.

Boc-A5c-OH was synthesized in an analogous manner to that of Boc-A6c-OH. Other protected Acc amino acids can be prepared in an analogous manner by a person of ordinary skill in the art as enabled by the teachings herein.

In the synthesis of a GLP-1 analogue of this invention containing A5c, A6c, Aib, Boc-Lys(2CIZ)-OH and/or Boc-His(DNP)-OH, the coupling time is 2 hrs. for these residues and the residue immediately following them. Otherwise the coupling time is approximately 5 minutes. For the synthesis of (Tma-His⁷)hGLP-1(7-36)NH₂(SEQ ID NO.778), HBTU (2 mmol) and DIEA (1.0 ml) in 4 ml DMF are used to react with the N-terminal free amine of the peptide-resin in the last coupling reaction; the coupling time is about 2 hours.

The substituents R² and R³ of the above generic formula can be attached to the free amine of the N-terminal amino acid by standard methods known in the art. For example, alkyl groups, e.g., (C₁-C₃₀)alkyl, can be attached using reductive alkylation. Hydroxyalkyl groups, e.g., (C₁-C₃₀)hydroxyalkyl, can also be attached using reductive alkylation wherein the free hydroxy group is protected with a t-butyl ester. Acyl groups, e.g., COX¹, can be attached by coupling the free acid, e.g., X¹COOH, to the free amine of the N-terminal amino acid by mixing the completed resin with 3 molar equivalents of both the free acid and diisopropylcarbodiimide in methylene chloride for about one hour. If the free acid contains a free hydroxy group, e.g., p-hydroxyphenylpropionic acid, then the coupling should be performed with an additional 3 molar equivalents of HOBT.

A compound of the present invention can be tested for activity as a GLP-1 binding compound according to the following procedure.

Cell Culture:

RIN 5F rat insulinoma cells (ATCC-# CRL-2058, American Type Culture Collection, Manassas, Va.), expressing the GLP-1 receptor, were cultured in Dulbecco's modified Eagle's medium (DMEM) containing 10% fetal calf serum, and maintained at about 37° C. in a humidifed atmosphere of 5% CO₂/95% air.

Radioligand Binding:

Membranes were prepared for radioligand binding studies by homogenization of the RIN cells in 20 ml of ice-cold 50 mM Tris-HCl with a Brinkman Polytron (Westbury, N.Y.) (setting 6, 15 sec). The homogenates were washed twice by centrifugation (39,000 g/10 min), and the final pellets were resuspended in 50 mM Tris-HCl, containing 2.5 mM MgCl₂, 0.1 mg/ml bacitracin (Sigma Chemical, St. Louis, Mo.), and 0.1% BSA. For assay, aliquots (0.4 ml) were incubated with 0.05 nM (¹²⁵I)GLP-1(7-36) (˜2200 Ci/mmol, New England Nuclear, Boston, Mass.), with and without 0.05 ml of unlabeled competing test peptides. After a 100 min incubation (25° C.), the bound (¹²⁵I)GLP-1(7-36) (SEQ ID NO: 775) was separated from the free by rapid filtration through GF/C filters (Brandel, Gaithersburg, Md.), which had been previously soaked in 0.5% polyethyleneimine. The filters were then washed three times with 5 ml aliquots of ice-cold 50 mM Tris-HCl, and the bound radioactivity trapped on the filters was counted by gamma spectrometry (Wallac LKB, Gaithersburg, Md.). Specific binding was defined as the total (¹²⁵I)GLP-1(7-36) (SEQ ID NO: 775) bound minus that bound in the presence of 1000 nM GLP1(7-36) (Bachem, Torrence, Calif.).

The peptides of this invention can be provided in the form of pharmaceutically acceptable salts. Examples of such salts include, but are not limited to, those formed with organic acids (e.g., acetic, lactic, maleic, citric, malic, ascorbic, succinic, benzoic, methanesulfonic, toluenesulfonic, or pamoic acid), inorganic acids (e.g., hydrochloric acid, sulfuric acid, or phosphoric acid), and polymeric acids (e.g., tannic acid, carboxymethyl cellulose, polylactic, polyglycolic, or copolymers of polylactic-glycolic acids). A typical method of making a salt of a peptide of the present invention is well known in the art and can be accomplished by standard methods of salt exchange. Accordingly, the TFA salt of a peptide of the present invention (the TFA salt results from the purification of the peptide by using preparative HPLC, eluting with TFA containing buffer solutions) can be converted into another salt, such as an acetate salt by dissolving the peptide in a small amount of 0.25 N acetic acid aqueous solution. The resulting solution is applied to a semi-prep HPLC column (Zorbax, 300 SB, C-8). The column is eluted with (1) 0.1N ammonium acetate aqueous solution for 0.5 hrs., (2) 0.25N acetic acid aqueous solution for 0.5 hrs. and (3) a linear gradient (20% to 100% of solution B over 30 min.) at a flow rate of 4 m/min (solution A is 0.25N acetic acid aqueous solution; solution B is 0.25N acetic acid in acetonitrile/water, 80:20). The fractions containing the peptide are collected and lyophilized to dryness.

As is well known to those skilled in the art, the known and potential uses of GLP-1 is varied and multitudinous (See, Todd, J. F., et al., Clinical Science, 1998, 95, pp. 325-329; and Todd, J. F. et al., European Journal of Clinical Investigation, 1997, 27, pp.533-536). Thus, the administration of the compounds of this invention for purposes of eliciting an agonist effect can have the same effects and uses as GLP-1 itself. These varied uses of GLP-1 may be summarized as follows, treatment of: Type I diabetes, Type II diabetes, obesity, glucagonomas, secretory disorders of the airway, metabolic disorder, arthritis, osteoporosis, central nervous system diseases, restenosis, neurodegenerative diseases, renal failure, congestive heart failure, nephrotic syndrome, cirrhosis, pulmonary edema, hypertension, and disorders wherein the reduction of food intake is desired. GLP-1 analogues of the present invention that elicit an antagonist effect from a subject can be used for treating the following: hypoglycemia and malabsorption syndrome associated with gastroectomy or small bowel resection.

Accordingly, the present invention includes within its scope pharmaceutical compositions comprising, as an active ingredient, at least one of the compounds of formula (I) in association with a pharmaceutically acceptable carrier.

The dosage of active ingredient in the compositions of this invention may be varied; however, it is necessary that the amount of the active ingredient be such that a suitable dosage form is obtained. The selected dosage depends upon the desired therapeutic effect, on the route of administration, and on the duration of the treatment. In general, an effective dosage for the activities of this invention is in the range of 1×10⁻⁷ to 200 mg/kg/day, preferably 1×10⁻⁴ to 100 mg/kg/day, which can be administered as a single dose or divided into multiple doses.

The compounds of this invention can be administered by oral, parenteral (e.g., intramuscular, intraperitoneal, intravenous or subcutaneous injection, or implant), nasal, vaginal, rectal, sublingual or topical routes of administration and can be formulated with pharmaceutically acceptable carriers to provide dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Compositions for rectal or vaginal administration are preferably suppositories which may contain, in addition to the active substance, excipients such as coca butter or a suppository wax.

Compositions for nasal or sublingual administration are also prepared with standard excipients well known in the art.

Further, a compound of this invention can be administered in a sustained release composition such as those described in the following patents and patent applications. U.S. Pat. No. 5,672,659 teaches sustained release compositions comprising a bioactive agent and a polyester. U.S. Pat. No. 5,595,760 teaches sustained release compositions comprising a bioactive agent in a gelable form. U.S. application Ser. No. 08/929,363 filed Sep. 9, 1997, teaches polymeric sustained release compositions comprising a bioactive agent and chitosan. U.S. application Ser. No. 08/740,778 filed Nov. 1, 1996, teaches sustained release compositions comprising a bioactive agent and cyclodextrin. U.S. application Ser. No. 09/015,394 filed Jan. 29, 1998, teaches absorbable sustained release compositions of a bioactive agent. U.S. application Ser. No. 09/121,653 filed Jul. 23, 1998, teaches a process for making microparticles comprising a therapeutic agent such as a peptide in an oil-in-water process. U.S. application Ser. No. 09/131,472 filed Aug. 10, 1998, teaches complexes comprising a therapeutic agent such as a peptide and a phosphorylated polymer. U.S. application Ser. No. 09/184,413 filed Nov. 2, 1998, teaches complexes comprising a therapeutic agent such as a peptide and a polymer bearing a non-polymerizable lactone. The teachings of the foregoing patents and applications are incorporated herein by reference.

Unless defined otherwise, all technical and scientific terms used herein have the same meaning as commonly understood by one of ordinary skill in the art to which this invention belongs. Also, all publications, patent applications, patents and other references mentioned herein are incorporated by reference.

Synthesis:

The following example describes a synthetic method for making a peptide of the invention, which method is well-known to those skilled in the art of peptide synthesis. Where provided, the amounts of reagents are approximately those employed to carry out the synthesis on a 0.20 mmol scale. Other methods are also well known to those skilled in the art, thus the example is provided for the purpose of illustration only and is not meant to limit the scope of the present Invention in any manner.

A great variety of amino acids, with and without side-chain protection, are available from a number of commercial sources. For example, the following amino acids, wherein parentheses designate the side-chain protecting group, if present, are variously available from Bachem, Torrance, Calif.; Nova Biochem., LaJolla, Calif.; Chem-Impex International; Wood Dale, Ill.; and Synthetech, Inc. Albany, Oreg.: Boc-Ado-OH, Boc-Aib-OH, Boc-Ala-OH, Boc-βAla-OH, Boc-Arg(Tos)-OH, Boc-D-Arg(Tos)-OH, Boc-Asp(OcHex)-OH, Boc-D-Asp(OcHex)-OH, Boc-Aun-OH, Boc-Ava-OH, Boc-Gln-OH, Boc-Glu(OcHex)-OH, Boc-Gly-OH, Boc-His(DNP)-OH, Boc-Ile-OH, Boc-Leu-OH, Boc-Lys(2CIZ)-OH, Boc-Nal-OH, Boc-Phe-OH, Boc-Ser(Bzl)-OH, Boc-Thr(Bzl)-OH, Boc-Trp(Fm)-OH, Boc-Tyr(2BrZ)-OH, and Boc-Val-OH.

### EXAMPLE 1

### Synthesis of (Ser⁸, Aib³⁵)hGLP-1(7-36)NH₂

The title peptide may be synthesized on an Applied Biosystems (Foster City, Calif.) model 430A peptide synthesizer modified to do accelerated Boc-chemistry solid phase peptide synthesis. (See Schnolzer, et al., Int. J. Peptide Protein Res., 90:180 (1992).) 4-Methylbenzhydrylamine (MBHA) resin (Peninsula Laboratories, Belmont, Calif.) with the substitution of 0.91 mmol/g may be used. Boc amino acids (Bachem, Calif., Torrance, Calif.; Nova Biochem., LaJolla, Calif.) may be used, e.g., with the following side chain protection: Boc-Ala-OH, Boc-Arg(Tos)-OH, Boc-Asp(OcHex)-OH, Boc-Tyr(2BrZ)-OH, Boc-His(DNP)-OH, Boc-Val-OH, Boc-Leu-OH, Boc-Gly-OH, Boc-Gln-OH, Boc-Ile-OH, Boc-Lys(2CIZ)-OH, Boc-Thr(Bzl)-OH, Boc-Ser(Bzl)-OH, Boc-Phe-OH, Boc-Glu(OcHex)-OH and Boc-Trp(Fm)-OH. The Boc groups are removed by treatment with 100% TFA for 2×1 min. Boc amino acids (2.5 mmol) are pre-activated with HBTU (2.0 mmol) and DIEA (1.0 mL) in 4 mL of DMF and are coupled without prior neutralization of the peptide-resin TFA salt.

At the end of the assembly of the peptide chain, the resin is treated with a solution of 20% mercaptoethanol/10% DIEA in DMF for 2×30 min. to remove the DNP group on the His side chain. The N-terminal Boc group is then removed by treatment with 100% TFA for 2×2 min. After neutralization of the peptide-resin with 10% DIEA in DMF (1×1 min), the formyl group on the side chain of Trp is removed by treatment with a solution of 15% ethanolamine/15% water/70% DMF for 2×30 min. The peptide-resin is then washed with DMF and DCM and dried under reduced pressure. The final cleavage is performed by stirring the peptide-resin in 10 mL of HF containing 1 mL of anisole and dithiothreitol (24 mg) at 0° C. for approximately 75 min. HF is removed by a flow of nitrogen and the residue is then washed with ether (6×10 mL) and extracted with 4N HOAc (6×10 mL).

The peptide mixture in the aqueous extract is purified on reverse-phase preparative high pressure liquid chromatography (HPLC) using a reverse phase VYDAC® C₁₈ column (Nest Group, Southborough, Mass.). The column is eluted with a linear gradient (20% to 50% of solution B over 105 min.) at a flow rate of 10 mL/min (Solution A=water containing 0.1% TFA; Solution B=acetonitrile containing 0.1% of TFA). Fractions are collected and checked on analytical HPLC. Those fractions containing purified product are then combined and lyophilized to dryness. Electro-spray mass spectrometer (MS(ES)) analysis may be used to check the molecular weight of the final product.

### EXAMPLES 2-7

The following peptides may also be synthesized according to the procedure detailed in Example 1, substituting appropriate amino acids during peptide assembly.

| | |
|---|---|
| (Abu⁸,β-Ala³⁵)hGLP-1(7-36)NH₂; | (SEQ ID NO.768) |
| (Val⁸,β-Ala³⁵)hGLP-1(7-36)NH₂; | (SEQ ID NO.769) |
| (β-Ala^{8,35})hGLP-1(7-36)NH₂; | (SEQ ID NO.770) |
| (Abu⁸,Aib³⁵)hGLP-1(7-36)NH₂; | (SEQ ID NO.771) |
| (Val⁸,Aib³⁵)hGLP-1(7-36)NH₂; | (SEQ ID NO.772) |
| and | |
| (β-Ala⁸,Aib³⁵)hGLP-1(7-36)NH₂; | (SEQ ID NO.773) |

Each of the foregoing examples can be made according to the procedures described hereinabove, with appropriate substitutions in starting materials during peptide assembly.

### Other Embodiments

From the above description, one skilled in the art can easily ascertain the essential characteristics of the present invention, and without departing from the spirit and scope thereof, can make various changes and modifications of the invention to adapt it to various uses and conditions. Thus, other embodiments are also within the claims.

What is claimed is:

1. A compound of formula (I),
(R²R³)-A⁷⁻A⁸-A⁹-A¹⁰-A¹¹-A¹²-A¹³-A¹⁴-A¹⁵-A¹⁶-A¹⁷⁻A¹⁸-A¹⁹-A²⁰-A²¹-A²²-A²³-A²⁴A²⁵-A²⁶-A²⁷⁻A²⁸-A²⁹-A³⁰-A³¹-A³²-A³³-A³⁴-A³⁵-A³⁶-A³⁷⁻A³⁸-A³⁹-R¹, (I)
wherein:
A⁷ is L-His, Ura, Paa, Pta, Amp, Tma-His, des-amino-His, or deleted;
A³ is Abu, β-Ala, Ser, Gly or Val;
A⁹ is Glu, N-Me-Glu, N-Me-Asp or Asp;
A¹⁰ is Gly, Acc, β-Ala or Aib;
A¹¹ is Thr or Ser;
A¹² is Phe, Acc, Aic, Aib, 3-Pal, 4-Pal, 1-Nal, 2-Nal, Cha, Trp or X¹-Phe;
A¹³ is Thr or Ser;
A¹⁴ is Ser or Aib;
A¹⁵ is Asp or Glu;
A¹⁶ is Val, Acc, Aib, Leu, Ile, Tle, Nle, Abu, Ala or Cha;
A¹⁷ is Ser or Thr;
A¹⁸ is Ser or Thr;
A¹⁹ is Tyr, Cha, Phe, 3-Pal, 4-Pal, Acc, 1-Nal, 2-Nal, or X¹-Phe;
A²⁰ is Leu, Acc, Aib, Nle, Ile, Cha, Tle, Val, Phe or X¹-Phe;
A²¹ is Glu or Asp;
A²² is Gly, Acc, β-Ala, Glu or Aib;
A²³ is Gin, Asp, Asn or Glu;
A²⁴ is Ala, Aib, Val, Abu, Tle or Acc;
A²⁵ is Ala, Aib, Val, Abu, Tle, Acc, Lys, Arg, hArg, Orn, HN—CH((CH₂)ₙ—N(R¹⁰R¹¹))—C(O) or HN—CH((CH₂)ₑ—X³)—C(O);
A²⁶ is Lys, Arg, hArg, Orn, HN—CH((CH₂)ₙ—N(R¹⁰R¹¹))—C(O) or HN—CH((CH₂)ₑ—X³C(O);
A²⁷ is Glu, Asp, Leu, Aib or Lys;
A²⁸ is Phe, 3-Pal, 4-Pal, 1-Nal, 2-Nal, X¹-Phe, Aic, Acc, Aib, Cha or Trp;
A²⁹ is Ile, Acc, Aib, Leu, Nle, Cha, Tle, Val, Abu, Ala or Phe;
A³⁰ is Ala, Aib or Acc;
A³¹ is Trp, 1-Nal, 2-Nal, 3-Pal, 4-Pal, Phe, Acc, Aib or Cha;
A³² is Leu, Acc, Aib, Nle, Ile, Cha, Tle, Phe, X¹-Phe or Ala;
A³³ is Val, Acc, Aib, Leu, Ile, Tle, Nle, Cha, Ala, Phe, Abu, Lys or X¹-Phe;
A³⁴ is Lys, Arg, hArg, Orn, HN—CH((CH₂)ₙ—N(R¹⁰R¹¹))—C(O) or HN—CH((CH₂)ₑ—X³)—C(O);
A³⁵ is Gly, β-Ala, D-Ala, Gaba, Ava, HN—(CH₂)ₘ—C(O), Aib, Acc or a D-amino acid;
A³⁶ is L- or D-Arg, D- or L-Lys, D- or L-hArg, D- or L-Orn, HN—CH((CH₂)ₙ—N(R¹⁰R¹¹))—C(O), HN—CH((CH₂)ₑ—X³)—C(O) or deleted;
A³⁷ is Gly, β-Ala, Gaba, Ava, Aib, Acc, Ado, Arg, Asp, Aun, Aec, HN—(CH₂)ₘ—C(O), HN—CH((CH₂)ₙ—N(R¹⁰R¹¹))—C(O), HN—CH((CH₂)ₙ—O(R¹⁰))—C(O), a D-amino acid, or deleted;
A³⁸ is D- or L-Lys, D- or L-Arg, D- or L-hArg, D- or L-Orn, HN—CH((CH₂)ₙ—N(R¹⁰R¹¹))—C(O), HN—CH((CH₂)ₑ—X³)—C(O) Ava, Ado, Aec or deleted;
A³⁹ is D- or L-Lys, D- or L-Arg, HN—CH((CH₂)ₙ—N(R¹⁰R¹¹))—C(O), Ava, Ado, Aec, or Aun, or is deleted;
X¹ is, independently for each occurrence, selected from the group consisting of (C₁-C₆)alkyl, OH and halo;
R¹ is OH, NH₂, (C₁-C₃₀)alkoxy, or NH—X²—CH₂-Z⁰, wherein X² is a (C₁-C₁₂)hydrocarbon moiety, and Z⁰ is H, OH, CO₂H or CONH₂; 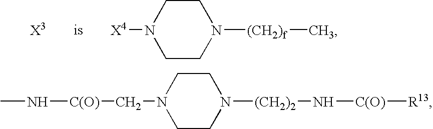
or —C(O)—NHR¹², wherein X⁴ is, independently for each occurrence, —C(O)—, —NH—C(O)— or —CH₂—, and wherein f is, independently for each occurrence, an integer from 1 to 29 inclusive;
each of R² and R³ is independently selected from the group consisting of H, (C₁-C₃₀)alkyl, (C₂-C₃₀)alkenyl, phenyl(C₁-C₃₀)alkyl, naphthyl(C₁-C₃₀)alkyl, hydroxy(C₁-C₃₀)alkyl, hydroxy(C₂-C₃₀)alkenyl, hydroxyphenyl(C₁-C₃₀)alkyl, and hydroxynaphthyl(C₁-C₃₀)alkyl; or one of R² and R³ is (CH₃)₂—N—C═N(CH₃)₂, (C₁-C₃₀)acyl, (C₁-C₃₀)alkylsulfonyl, C(O)X⁵, 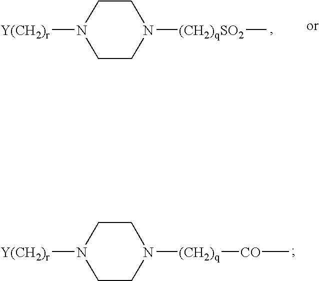
wherein Y is H, OH or NH₂; r is 0 to 4; q is 0 to 4; and X⁵ is (C₁-C₃₀)alkyl, (C₂-C30)alkenyl, phenyl(C₁-C₃₀)alkyl, naphthyl(C₁-C₃₀)alkyl, hydroxy(C₁-C₃₀)alkyl, hydroxy(C₂-C₃₀)alkenyl, hydroxyphenyl(C₁-C₃₀)alkyl or hydroxynaphthyl(C₁-C₃₀)alkyl;
e is, independently for each occurrence, an integer from 1 to 4 inclusive;
m is, independently for each occurrence, an integer from 5 to 24 inclusive;
n is, independently for each occurrence, an integer from 1 to 5, inclusive;
each of R¹⁰ and R¹¹ is, independently for each occurrence, H, (C₁-C₃₀)alkyl, (C₁-C₃₀)acyl, (C₁-C₃₀)alkylsulfonyl, —C((NH)(NH₂)) or 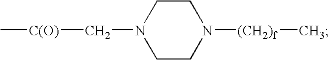
and R¹² and R¹³ each is, independently for each occurrence, (C₁-C₃₀)alkyl; provided that:
(i) said compound is not:
(Ser^{8,}β-Ala³⁵)hGLP-1(7-36)NH₂(SEQ ID NO: 779);
(Gly⁸, Aib³⁵)hGLP-1(7-36)NH₂(SEQ ID NO: 780); or
(Gly³, β-Ala³⁵)hGLP-1(7-36)NH₂(SEQ ID NO: 781);
(ii) when A⁷ is Ura, Paa or Pta, then R² and R³ are deleted; and
(iii) when R¹⁰ is (C₁-C₃₀)acyl, (C₁-C₃₀)alkylsulfonyl, —C((NH)(NH₂)) or 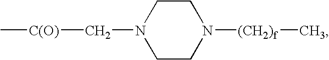
then R¹¹ is H or (C₁-C₃₀)alkyl; or a pharmaceutically acceptable salt thereof.

2. A compound according to claim 1, wherein A¹¹ is Thr; A¹³ is Thr; A¹⁵ is Asp; A¹⁷ is Ser; A¹⁸ is Ser; A²¹ is Glu; A²³ is Gln or Glu; A²⁷ is Glu; and A³¹ is Trp; or a pharmaceutically acceptable salt thereof.

3. A compound according to claim 2, wherein A⁹ is Glu, N-Me-Glu or N-Me-Asp; A¹² is Phe, Acc or Aic; A¹⁶ is Val, Acc or Aib; A¹⁹ is Tyr; A²⁰ is Leu, Acc or Cha; A²⁴ is Ala, Aib or Acc; A² is Ala, Aib, Acc, Lys, Arg, hArg, Orn, HN—CH((CH₂)ₙ—N(R¹⁰R¹¹))—C(O) or HN—CH((CH₂)ₑ—X³)—C(O); A²⁸ is Phe; A²⁹ is Ile or Acc; A³⁰ is Ala or Aib; A³² is Leu, Acc or Cha; and A³³ is Val or Acc; or a pharmaceutically acceptable salt thereof.

4. A compound according to claim 3, wherein A¹⁰ is Gly; A¹² is Phe, A6c or A5c; A¹⁶ is Val, A6c or A5c; A²⁰ is Leu, A6c, A5c or Cha; A²² is Gly, β-Ala or Aib; A²⁴ is Ala or Aib; A²⁹ is Ile, A6c or A5c; A³² is Leu, A6c, A5c or Cha; A³³ is Val, A6c or A5c; A³⁵ is Aib, β-Ala, Ado, A6c, A5c or Gly; and A³⁷ is Gly, Aib, β-Ala, Ado, D-Ala or deleted; or a pharmaceutically acceptable salt thereof.

5. A compound according to claim 4, wherein X⁴ for each occurrence is —C(O)—; e for each occurrence is independently 1 or 2; and R¹ is OH or NH₂; or a pharmaceutically acceptable salt thereof.

6. A compound according to claim 5, wherein R² is H and R³ is (C₁-C₃₀)alkyl, (C₂-C₃₀)alkenyl, (C₁-C₃₀)acyl, (C₁-C₃₀)alkylsulfonyl, 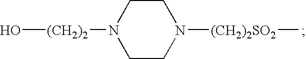 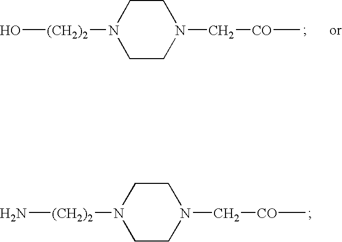
or a pharmaceutically acceptable salt thereof.

7. A compound according to claim 5, wherein R¹⁰ is (C₁-C₃₀)acyl, (C₁-C₃₀)alkylsulfonyl or 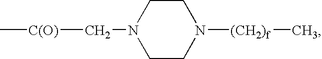
and R¹¹ is H; or a pharmaceutically acceptable salt thereof

8. A compound according to claim 7, wherein R¹⁰ is (C₄-C₂₀)acyl, (C₄-C₂₀)alkylsulfonyl or 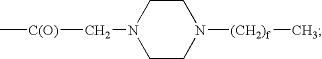
or a pharmaceutically acceptable salt thereof.

9. A compound according to claim 5, wherein A⁸ is Gly, Ser, or Val; or a pharmaceutically acceptable salt thereof.

10. A compound according to claim 9 wherein A⁸ is Gly or Ser; or a pharmaceutically acceptable salt thereof.

11. A compound according to claim 10, wherein said compound is according to the formula:

| | |
|---|---|
| ((N^{α}-Me-His)⁷,Gly⁸,Aib³⁵)hGLP-1(7-36)NH₂; | (SEQ ID NO.1) |
| ((N^{α}-Me-His)⁷,Gly⁸,β-Ala³⁵)hGLP-1(7-36)NH₂; | (SEQ ID NO.2) |
| ((N^{α}-Me-His)⁷,Gly⁸,Aib³⁵,Arg^{26,34})hGLP-1(7-36)NH₂; | (SEQ ID NO.3) |
| ((N^{α}-Me-His)⁷,Gly⁸,Arg^{26,34},β-Ala³⁵)hGLP-1(7-36)NH₂; | (SEQ ID NO.4) |
| (Gly⁸,A6c³⁵)hGLP-1(7-36)NH₂; | (SEQ ID NO.5) |
| (Gly⁸,A5c³⁵)hGLP-1(7-36)NH₂: | (SEQ ID NO.6) |
| (Gly⁸,D-Ala³⁵)hGLP-1(7-36)NH₂; (SEQ ID NO.7) | |
| (Gly⁸,Aib³⁵,A6c³²)hGLP-1(7-36)NH₂; | (SEQ ID NO.8) |
| (Gly⁸,Aib³⁵,A5c³²)hGLP-1(7-36)NH₂; | (SEQ ID NO.9) |
| (Gly⁸,Aib³⁵,Glu²³)hGLP-1(7-36)NH₂; | (SEQ ID NO.10) |
| (Gly⁸,Aib^{24,35})hGLP-1(7-36)NH₂; | (SEQ ID NO.11) |
| (Gly⁸,Aib^{30,35})hGLP-1(7-36)NH₂; | (SEQ ID NO.12) |
| (Gly⁸,Aib^{25,35})hGLP-1(7-36)NH₂; | (SEQ ID NO.13) |
| (Gly⁸,Aib³⁵,A6c^{16,20})hGLP-1(7-36)NH₂; | (SEQ ID NO.14) |
| (Gly⁸,Aib³⁵,A6c^{16,29,32})hGLP-1(7-36)NH₂; | (SEQ ID NO.15) |
| (Gly⁸,Aib³⁵,A6c^{20,32})hGLP-1(7-36)NH₂; | (SEQ ID NO.16) |
| (Gly⁸,Aib³⁵,A6c²⁰)hGLP-1(7-36)NH₂; | (SEQ ID NO.17) |
| (Gly⁸,Aib³⁵,Lys²⁵)hGLP-1(7-36)NH₂; | (SEQ ID NO.18) |
| (Gly⁸,Aib^{24,35},A6c²⁰)hGLP-1(7-36)NH₂; | (SEQ ID NO.19) |
| (Gly⁸,Aib³⁵,A6c^{29,32})hGLP-1(7-36)NH₂; | (SEQ ID NO.20) |
| (Gly⁸,Aib^{24,35},A6c^{29,32})hGLP-1(7-36)NH₂; | (SEQ ID NO.21) |
| (Gly⁸,Aib³⁵,A6c¹²)hGLP-1(7-36)NH₂; | (SEQ ID NO.22) |
| (Gly⁸,Aib³⁵,Cha²⁰)hGLP-1(7-36)NH₂; | (SEQ ID NO.23) |
| (Gly⁸,Aib³⁵,A6c³³)hGLP-1(7-36)NH₂; | (SEQ ID NO.24) |
| (Gly⁸,A6c^{16,20},β-Ala³⁵)hGLP-1(7-36)NH₂; | (SEQ ID NO.25) |
| (Gly⁸,Aib³⁵,β-Ala²²)hGLP-1(7-36)NH₂; | (SEQ ID NO.26) |
| (Gly⁸,Aib^{22,35})hGLP-1(7-36)NH₂; | (SEQ ID NO.27) |
| (Gly⁸,Aib³⁵,Glu²³,A6c³²)hGLP-1(7-36)NH₂; | (SEQ ID NO.28) |
| (Gly⁸,Aib^{24,35},Glu²³,A6c³²)hGLP-1(7-36)NH₂; | (SEQ ID NO.29) |
| (Gly⁸,Aib^{24,25,35},Glu²³,A6c³²)hGLP-1(7-36)NH₂; | (SEQ ID NO.30) |
| (Gly⁸,Aib^{24,25,35},A6c^{16,20,32},Glu²³)hGLP-1(7-36)NH₂; | (SEQ ID NO.31) |
| (Gly⁸,A6c³²,β-Ala³⁵)hGLP-1(7-36)NH₂; | (SEQ ID NO.32) |
| (Gly⁸,A5c³²,β-Ala³⁵)hGLP-1(7-36)NH₂; | (SEQ ID NO.33) |
| (Gly⁸,Glu²³,β-Ala³⁵)hGLP-1(7-36)NH₂; | (SEQ ID NO.34) |
| (Gly⁸,Aib²⁴,β-Ala³⁵)hGLP-1(7-36)NH₂; | (SEQ ID NO.35) |
| (Gly⁸,Aib³⁰,β-Ala³⁵)hGLP-1(7-36)NH₂; | (SEQ ID NO.36) |
| (Gly⁸,Aib²⁵,β-Ala³⁵)hGLP-1(7-36)NH₂; | (SEQ ID NO.37) |
| (Gly⁸,A6c^{16,29,32},β-Ala³⁵)hGLP-1(7-36)NH₂; | (SEQ ID NO.38) |
| (Gly⁸,A6c^{20,32},β-Ala³⁵)hGLP-1(7-36)NH₂; | (SEQ ID NO.39) |
| (Gly⁸,A6c²⁰,β-Ala³⁵)hGLP-1(7-36)NH₂; | (SEQ ID NO.40) |
| (Gly⁸,Lys²⁵,β-Ala³⁵)hGLP-1(7-36)NH₂; | (SEQ ID NO.41) |
| (Gly⁸,Aib²⁴,A6c²⁰,β-Ala³⁵)hGLP-1(7-36)NH₂; | (SEQ ID NO.42) |
| (Gly⁸,A6c^{29,32},β-Ala³⁵)hGLP-1(7-36)NH₂; | (SEQ ID NO.43) |
| (Gly⁸,Aib²⁴,A6c^{29,32},β-Ala³⁵)hGLP-1(7-36)NH₂; | (SEQ ID NO.44) |
| (Gly⁸,A6c¹²,β-Ala³⁵)hGLP-1(7-36)NH₂; | (SEQ ID NO.45) |
| (Gly⁸,Cha²⁰,β-Ala³⁵)hGLP-1(7-36)NH₂; | (SEQ ID NO.46) |
| (Gly⁸,A6c³³,β-Ala³⁵)hGLP-1(7-36)NH₂; | (SEQ ID NO.47) |
| (Gly⁸,β-Ala^{22,35})hGLP-1(7-36)NH₂; | (SEQ ID NO.48) |
| (Gly⁸,Aib²²,β-Ala³⁵)hGLP-1(7-36)NH₂; | (SEQ ID NO.49) |
| (Gly⁸,Glu²³,A6c³²,β-Ala³⁵)hGLP-1(7-36)NH₂; | (SEQ ID NO.50) |
| (Gly⁸,Aib²⁴,Glu²³,A6c³²,β-Ala³⁵)hGLP-1(7-36)NH₂; | (SEQ ID NO.51) |
| (Gly⁸,Aib²⁴,Glu²³,A6c³²,Lys³⁴(N^{e}-octanoyl),β-Ala³⁵) | (SEQ ID NO.52) |
| hGLP-1(7-36)NH₂; | |
| (Gly⁸,Aib^{24,25},Glu²³,A6c³²,β-Ala³⁵)hGLP-1(7-36)NH₂; | (SEQ ID NO.53) |
| (Gly⁸,Aib^{24,25},A6c^{16,20,32},Glu²³,β-Ala³⁵)hGLP-1(7-36)NH₂; | (SEQ ID NO.54) |
| (Gly⁸,Aib³⁵,D-Arg³⁶)hGLP-1(7-36)NH₂; | (SEQ ID NO.55) |
| (Gly⁸,Aib³⁵,D-Lys³⁶)hGLP-1(7-36)NH₂; | (SEQ ID NO.56) |
| (Gly⁸,β-Ala³⁵,D-Arg³⁶)hGLP-1(7-36)NH₂; | (SEQ ID NO.57) |
| (Gly⁸,β-Ala³⁵,D-Lys³⁶)hGLP-1(7-36)NH₂; | (SEQ ID NO.58) |
| (Gly⁸,Aib³⁵,Arg^{26,34})hGLP-1(7-36)NH₂; | (SEQ ID NO.59) |
| (Gly⁸,Arg^{26,34},β-Ala³⁵)hGLP-1(7-36)NH₂; | (SEQ ID NO.60) |
| (Gly⁸,Aib³⁵,Arg^{25,26,34})hGLP-1(7-36)NH₂; | (SEQ ID NO.61) |
| (Gly⁸,Arg^{25,26,34},β-Ala³⁵)hGLP-1(7-36)NH₂; | (SEQ ID NO.62) |
| (Gly⁸,Arg^{26,34},β-Ala³⁵,Lys³⁶(N^{ε}-tetradecanoyl))hGLP-1(7-36)OH; | (SEQ ID NO.63) |
| (Gly⁸,Aib³⁵,Arg^{26,34},Lys³⁶(N^{ε}-tetradecanoyl))hGLP-1(7-37)OH; | (SEQ ID NO.64) |
| (Gly⁸,Aib^{35,37},Arg^{26,34},Lys³⁶(N^{ε}-tetradecanoyl))hGLP-1(7-37)OH; | (SEQ ID NO.65) |
| (Gly⁸,Aib³⁵,Arg^{26,34},Lys³⁸(N^{ε}-tetradecanoyl),D-Ala³⁷) | (SEQ ID NO.66) |
| hGLP-1(7-37)OH; | |
| (Gly⁸,Aib^{35,37},Arg^{26,34},Lys³⁸(N^{ε}-tetradecanoyl))hGLP-1(7-38)OH; | (SEQ ID NO.67) |
| (Gly⁸,Aib³⁵,Arg^{26,34},β-Ala³⁷,Lys³⁸(N^{ε}-tetradecanoyl)) | (SEQ ID NO.68) |
| hGLP-1(7-38)OH; | |
| (Gly⁸,Aib³⁵,Arg^{26,34},Lys³⁹(N^{ε}-tetradecanoyl))hGLP-1(7-38)OH; | (SEQ ID NO.69) |
| (Gly⁸,Arg^{26,34},Lys³⁶(N^{ε}-tetradecanoyl),β-Ala³⁷)hGLP-1(7-37)OH; | (SEQ ID NO.70) |
| (Gly⁸,Aib³⁷,Arg^{26,34},Lys³⁶(N^{ε}-tetradecanoyl))hGLP-1(7-37)OH; | (SEQ ID NO.71) |
| (Gly⁸,Aib³⁵,Arg^{26,34},Ado³⁷)hGLP-1(7-37)OH; | (SEQ ID NO.72) |
| (Gly⁸,Aib³⁵,Arg^{26,34},Ado³⁷)hGLP-1(7-37)NH₂; | (SEQ ID NO.73) |
| (Gly⁸,Arg^{26,34},Lys³⁶(N^{ε}-tetradecanoyl),D-Ala³⁷)hGLP-1(7-37)OH; | (SEQ ID NO.74) |
| (Gly⁸,Aib³⁷,Arg^{26,34},Lys³⁸(N^{ε}-tetradecanoyl))hGLP-1(7-38)OH; | (SEQ ID NO.75) |
| (Gly⁸,Arg^{26,34},β-Ala³⁷,Lys³⁸(N^{ε}-tetradecanoyl))hGLP-1(7-38)OH; | (SEQ ID NO.76) |
| (Gly⁸,Aib³⁵,Lys²⁶(N^{ε}-octanoyl))hGLP-1(7-36)NH₂; | (SEQ ID NO.77) |
| (Gly⁸,Aib³⁵,Lys²⁶(N^{ε}-tetradecanoyl))hGLP-1(7-36)NH₂; | (SEQ ID NO.78) |
| (Gly⁸,Aib³⁵,Lys²⁶(N^{ε}-hexadecanoyl))hGLP-1(7-36)NH₂; | (SEQ ID NO.79) |
| (Gly⁸,Lys²⁶(N^{ε}-octanoyl),β-Ala³⁵)hGLP-1(7-36)NH₂; | (SEQ ID NO.80) |
| (Gly⁸,Lys²⁶(N^{ε}-tetradecanoyl),β-Ala³⁵)hGLP-1(7-36)NH₂; | (SEQ ID NO.81) |
| (Gly⁸,Lys²⁶(N^{ε}-hexadecanoyl),β-Ala³⁵)hGLP-1(7-36)NH₂; | (SEQ ID NO.82) |
| (Gly⁸,Aib³⁵,Lys²⁶(N^{ε}-octanoyl),Arg³⁴)hGLP-1(7-36)NH₂; | (SEQ ID NO.83) |
| (Gly⁸,Aib³⁵,Lys²⁶(N^{ε}-tetradecanoyl),Arg³⁴)hGLP-1(7-36)NH₂; | (SEQ ID NO.84) |
| (Gly⁸,Aib³⁵,Lys²⁶(N^{ε}-hexadecanoyl),Arg³⁴)hGLP-1(7-36)NH₂; | (SEQ ID NO.85) |
| (Gly⁸,Aib³⁵,Lys²⁶(N^{ε}-decanoyl),Arg³⁴)hGLP-1(7-36)NH₂; | (SEQ ID NO.86) |
| (Gly⁸,Aib³⁵,Lys²⁵,Lys²⁶(N^{ε}-octanoyl),Arg³⁴)hGLP-1(7-36)NH₂; | (SEQ ID NO.87) |
| (Gly⁸,Aib³⁵,Lys²⁵,Lys²⁶(N^{ε}-tetradecanoyl),Arg³⁴)hGLP-1(7-36)NH₂; | (SEQ ID NO.88) |
| (Gly⁸,Aib³⁵,Lys²⁵,Lys²⁶(N^{ε}-hexadecanoyl),Arg³⁴)hGLP-1(7-36)NH₂; | (SEQ ID NO.89) |
| (Gly⁸,Aib³⁵,Arg^{25,34},Lys²⁶(N^{ε}-octanoyl))hGLP-1(7-36)NH₂; | (SEQ ID NO.90) |
| (Gly⁸,Aib³⁵,Arg^{25,34},Lys²⁶(N^{ε}-tetradecanoyl))hGLP-1(7-36)NH₂; | (SEQ ID NO.91) |
| (Gly⁸,Aib³⁵,Arg^{25,34},Lys²⁶(N^{ε}-hexadecanoyl))hGLP-1(7-36)NH₂; | (SEQ ID NO.92) |
| (Gly⁸,Aib³⁵,Arg^{25,34},Lys²⁶(N^{ε}-decanoyl))hGLP-1(7-36)NH₂; | (SEQ ID NO.93) |
| (Gly⁸,Lys²⁶(N^{ε}-octanoyl),Arg³⁴,β-Ala³⁵)hGLP-1(7-36)NH₂; | (SEQ ID NO.94) |
| (Gly⁸,Lys²⁶(N^{ε}-tetradecanoyl),Arg³⁴,β-Ala³⁵)hGLP-1(7-36)NH₂; | (SEQ ID NO.95) |
| (Gly⁸,Lys²⁶(N^{ε}-hexadecanoyl),Arg³⁴,β-Ala³⁵)hGLP-1(7-36)NH₂; | (SEQ ID NO.96) |
| (Gly⁸,Lys²⁶(N^{ε}-decanoyl),Arg³⁴,β-Ala³⁵)hGLP-1(7-36)NH₂; | (SEQ ID NO.97) |
| (Gly⁸,Aib³⁵,Lys³⁴(N^{ε}-octanoyl))hGLP-1(7-36)NH₂; | (SEQ ID NO.98) |
| (Gly⁸,Aib³⁵,Lys³⁴(N^{ε}-tetradecanoyl))hGLP-1(7-36)NH₂; | (SEQ ID NO.99) |
| (Gly⁸,Aib³⁵,Lys³⁴(N^{ε}-hexadecanoyl))hGLP-1(7-36)NH₂; | (SEQ ID NO.100) |
| (Gly⁸,Aib³⁵,Arg²⁶,Lys³⁴(N^{ε}-octanoyl))hGLP-1(7-36)NH₂; | (SEQ ID NO.101) |
| (Gly⁸,Aib³⁵,Arg²⁶,Lys³⁴(N^{ε}-hexadecanoyl))hGLP-1(7-36)NH₂; | (SEQ ID NO.102) |
| (Gly⁸,Aib³⁵,Arg²⁶,Lys³⁴(N^{ε}-decanoyl))hGLP-1(7-36)NH₂; | (SEQ ID NO.103) |
| (Gly⁸,Aib³⁵,Arg^{25,26},Lys³⁴(N^{ε}-octanoyl))hGLP-1(7-36)NH₂; | (SEQ ID NO.104) |
| (Gly⁸,Aib³⁵,Arg^{25,26},Lys³⁴(N^{ε}-tetradecanoyl))hGLP-1(7-36)NH₂; | (SEQ ID NO.105) |
| (Gly⁸,Aib³⁵,Arg^{25,26},Lys³⁴(N^{ε}-hexadecanoyl))hGLP-1(7-36)NH₂; | (SEQ ID NO.106) |
| (Gly⁸,Aib³⁵,Arg^{25,26},Lys³⁴(N^{ε}-decanoyl))hGLP-1(7-36)NH₂; | (SEQ ID NO.107) |
| (Gly⁸,Aib³⁵,Lys²⁵,Arg²⁶,Lys³⁴(N^{ε}-octanoyl))hGLP-1(7-36)NH₂; | (SEQ ID NO.108) |
| (Gly⁸,Aib³⁵,Lys²⁵,Arg²⁶,Lys³⁴(N^{ε}-tetradecanoyl))hGLP-1(7-36)NH₂; | (SEQ ID NO.109) |
| (Gly⁸,Aib³⁵,Lys²⁵,Arg²⁶,Lys³⁴(N^{ε}-hexadecanoyl))hGLP-1(7-36)NH₂; | (SEQ ID NO.110) |
| (Gly⁸,Aib³⁵,Lys³⁶(N^{ε}-octanoyl))hGLP-1(7-36)NH₂; | (SEQ ID NO.111) |
| (Gly⁸,Aib³⁵,Lys³⁶(N^{ε}-tetradecanoyl))hGLP-1(7-36)NH₂; | (SEQ ID NO.112) |
| (Gly⁸,Aib³⁵,Lys³⁶(N^{ε}-hexadecanoyl))hGLP-1(7-36)NH₂; | (SEQ ID NO.113) |
| (Gly⁸,Aib³⁵,Arg²⁶,Lys³⁶(N^{ε}-octanoyl))hGLP-1(7-36)NH₂; | (SEQ ID NO.114) |
| (Gly⁸,Aib³⁵,Arg²⁶,Lys³⁶(N^{ε}-tetradecanoyl))hGLP-1(7-36)NH₂; | (SEQ ID NO.115) |
| (Gly⁸,Aib³⁵,Arg²⁶,Lys³⁶(N^{ε}-hexadecanoyl))hGLP-1(7-36)NH₂; | (SEQ ID NO.116) |
| (Gly⁸,Aib³⁵,Arg^{26,34},Lys³⁶(N^{ε}-octanoyl))hGLP-1(7-36)NH₂; | (SEQ ID NO.117) |
| (Gly⁸,Aib³⁵,Arg^{26,34},Lys³⁶(N^{ε}-hexadecanoyl))hGLP-1(7-36)NH₂; | (SEQ ID NO.118) |
| (Gly⁸,Aib³⁵,Arg^{26,34},Lys³⁸(N^{ε}-octanoyl))hGLP-1(7-38)NH₂; | (SEQ ID NO.119) |
| (Gly⁸,Aib³⁵,Arg^{26,34},Lys³⁸(N^{ε}-decanoyl))hGLP-1(7-38)NH₂; | (SEQ ID NO.120) |
| (Gly⁸,Aib³⁵,Arg^{26,34},Lys³⁸(N^{ε}-tetradecanoyl))hGLP-1(7-38)NH₂; | (SEQ ID NO.121) |
| (Gly⁸,Aib³⁵,Arg^{26,34},Lys³⁸(N^{ε}-hexadecanoyl))hGLP-1(7-38)NH₂; | (SEQ ID NO.122) |
| (Gly⁸,Aib^{35,37},Arg^{25,26,34},Lys³⁸(N^{ε}-octanoyl))hGLP-1(7-38)NH₂; | (SEQ ID NO.123) |
| (Gly⁸,Aib^{35,37},Arg^{25,26,34},Lys³⁸(N^{ε}-decanoyl))hGLP-1(7-38)NH₂; | (SEQ ID NO.124) |
| (Gly⁸,Aib^{35,37},Arg^{25,26,34},Lys³⁸(N^{ε}-tetradecanoyl))hGLP-1(7-38)NH₂; | (SEQ ID NO.125) |
| (Gly⁸,Aib^{35,37},Arg^{25,26,34},Lys³⁸(N^{ε}-hexadecanoyl))hGLP-1(7-38)NH₂; | (SEQ ID NO.126) |
| (Gly⁸,Aib^{35,37},Arg^{26,34},Lys³⁸(N^{ε}-octanoyl))hGLP-1(7-38)NH₂; | (SEQ ID NO.127) |
| (Gly⁸,Aib^{35,37},Arg^{26,34},Lys³⁸(N^{ε}-decanoyl))hGLP-1(7-38)NH₂; | (SEQ ID NO.128) |
| (Gly⁸,Aib^{35,37},Arg^{26,34},Lys³⁸(N^{ε}-hexadecanoyl))hGLP-1(7-38)NH₂; | (SEQ ID NO.129) |
| (Gly⁸,Aib³⁵,Lys²⁵,Arg^{26,34},Lys³⁶(N^{ε}-octanoyl))hGLP-1(7-36)NH₂; | (SEQ ID NO.130) |
| (Gly⁸,Aib³⁵,Lys²⁵,Arg^{26,34},Lys³⁶(N^{ε}-tetradecanoyl)) | (SEQ ID NO.131) |
| hGLP-1(7-36)NH₂; | |
| (Gly⁸,Aib³⁵,Lys²⁵,Arg^{26,34},Lys³⁶(N^{ε}-hexadecanoyl)) | (SEQ ID NO.132) |
| hGLP-1(7-36)NH₂; | |
| (Gly⁸,Aib³⁵,Arg^{25,26,34},Lys³⁶(N^{ε}-octanoyl))hGLP-1(7-36)NH₂; | (SEQ ID NO.133) |
| (Gly⁸,Aib³⁵,Arg^{25,26,34},Lys³⁶(N^{ε}-tetradecanoyl))hGLP-1(7-36)NH₂; | (SEQ ID NO.134) |
| (Gly⁸,Aib³⁵,Arg^{25,26,34},Lys³⁶(N^{ε}-hexadecanoyl))hGLP-1(7-36)NH₂; | (SEQ ID NO.135) |
| (Gly⁸,Aib³⁵,Arg^{25,26,34},Lys³⁵(N^{ε}-decanoyl))hGLP-1(7-36)NH₂; | (SEQ ID NO.136) |
| (Gly⁸,Lys³⁴(N^{ε}-octanoyl),β-Ala³⁵)hGLP-1(7-36)NH₂; | (SEQ ID NO.137) |
| (Gly⁸,Lys³⁴(N^{ε}-tetradecanoyl),β-Ala³⁵)hGLP-1(7-36)NH₂; | (SEQ ID NO.138) |
| (Gly⁸,Lys³⁴(N^{ε}-hexadecanoyl),β-Ala³⁵)hGLP-1(7-36)NH₂; | (SEQ ID NO.139) |
| (Gly⁸,A6c³²,Lys³⁴(N^{e}-octanoyl),β-Ala³⁵)hGLP-1(7-36)NH₂; | (SEQ ID NO.140) |
| (Gly⁸,Glu²³,Lys³⁴(N^{e}-octanoyl),β-Ala³⁵)hGLP-1(7-36)NH₂; | (SEQ ID NO.141) |
| (Gly⁸,Glu²³,A6c³²,Lys³⁴(N^{e}-octanoyl),β-Ala³⁵)hGLP-1(7-36)NH₂; | (SEQ ID NO.142) |
| (Gly⁸,Arg²⁶,Lys³⁴(N^{ε}-octanoyl),β-Ala³⁵)hGLP-1(7-36)NH₂; | (SEQ ID NO.143) |
| (Gly⁸,Arg²⁶,Lys³⁴(N^{ε}-tetradecanoyl),(β-Ala³⁵)hGLP-1(7-36)NH₂; | (SEQ ID NO.144) |
| (Gly⁸,Arg²⁶,Lys³⁴(N^{ε}-hexadecanoyl),β-Ala³⁵)hGLP-1(7-36)NH₂; | (SEQ ID NO.145) |
| (Gly⁸,Arg²⁶,Lys³⁴(N^{ε}-decanoyl),β-Ala³⁵)hGLP-1(7-36)NH₂; | (SEQ ID NO.146) |
| (Gly⁸,Arg^{25,26},Lys³⁴(N^{ε}-octanoyl),β-Ala³⁵)hGLP-1(7-36)NH₂; | (SEQ ID NO.147) |
| (Gly⁸,Arg^{25,26},Lys³⁴(N^{ε}-tetradecanoyl),β-Ala³⁵)hGLP-1(7-36)NH₂; | (SEQ ID NO.148) |
| (Gly⁸,Arg^{25,26},Lys³⁴(N^{ε}-hexadecanoyl),β-Ala³⁵)hGLP-1(7-36)NH₂; | (SEQ ID NO.149) |
| (Gly⁸,Arg^{25,26},Lys³⁴(N^{ε}-decanoyl),β-Ala³⁵)hGLP-1(7-36)NH₂; | (SEQ ID NO.150) |
| (Gly⁸,Lys²⁵,Arg²⁶,Lys³⁴(N^{ε}-octanoyl),β-Ala³⁵)hGLP-1(7-36)NH₂; | (SEQ ID NO.151) |
| (Gly⁸,Lys²⁵,Arg²⁶,Lys³⁴(N^{ε}-tetradecanoyl),β-Ala³⁵) | (SEQ ID NO.152) |
| hGLP-1(7-36)NH₂; | |
| (Gly⁸,Lys²⁵,Arg²⁶,Lys³⁴(N^{ε}-hexadecanoyl),β-Ala³⁵) | (SEQ ID NO.153) |
| hGLP-1(7-36)NH₂; | |
| (Gly⁸,β-Ala³⁵,Lys³⁶(N^{ε}-octanoyl))hGLP-1(7-36)NH₂; | (SEQ ID NO.154) |
| (Gly⁸,β-Ala³⁵,Lys³⁶(N^{ε}-tetradecanoyl))hGLP-1(7-36)NH₂; | (SEQ ID NO.155) |
| (Gly⁸,β-Ala³⁵,Lys³⁶(N^{ε}-hexadecanoyl))hGLP-1(7-36)NH₂; | (SEQ ID NO.156) |
| (Gly⁸,Arg²⁶,β-Ala³⁵,Lys³⁶(N^{ε}-octanoyl))hGLP-1(7-36)NH₂; | (SEQ ID NO.157) |
| (Gly⁸,Arg²⁶,β-Ala³⁵,Lys³⁶(N^{ε}-tetradecanoyl))hGLP-1(7-36)NH₂; | (SEQ ID NO.158) |
| (Gly⁸,Arg²⁶,β-Ala³⁵,Lys³⁶(N^{ε}-hexadecanoyl))hGLP-1(7-36)NH₂; | (SEQ ID NO.159) |
| (Gly⁸,Arg^{26,34},β-Ala³⁵,Lys³⁶(N^{ε}-octanoyl))hGLP-1(7-36)NH₂; | (SEQ ID NO.160) |
| (Gly⁸,Arg^{26,34},β-Ala³⁵,Lys³⁶(N^{ε}-tetradecanoyl))hGLP-1(7-36)NH₂; | (SEQ ID NO.161) |
| (Gly⁸,Arg^{26,34},β-Ala³⁵,Lys³⁶(N^{ε}-hexadecanoyl))hGLP-1(7-36)NH₂; | (SEQ ID NO.162) |
| (Gly⁸,Arg^{26,34},β-Ala³⁵,Lys³⁶(N^{ε}-decanoyl))hGLP-1(7-36)NH₂; | (SEQ ID NO.163) |
| (Gly⁸,Lys²⁵,Arg^{26,34},β-Ala³⁵,Lys³⁶(N^{ε}-octanoyl))hGLP-1(7-36)NH₂; | (SEQ ID NO.164) |
| (Gly⁸,Lys²⁵,Arg^{26,34},Lys³⁶(N^{ε}-tetradecanoyl),β-Ala³⁵) | (SEQ ID NO.165) |
| hGLP-1(7-36)NH₂; | |
| (Gly⁸,Lys²⁵,Arg^{26,34},β-Ala³⁵,Lys³⁶(N^{ε}-hexadecanoyl)) | (SEQ ID NO.166) |
| hGLP-1(7-36)NH₂; | |
| (Gly⁸,Arg^{25,26,34},β-Ala³⁵,Lys³⁶(N^{ε}-octanoyl))hGLP-1(7-36)NH₂; | (SEQ ID NO.167) |
| (Gly⁸,Arg^{25,26,34},β-Ala³⁵,Lys³⁶(N^{ε}-tetradecanoyl))hGLP-1(7-36)NH₂; | (SEQ ID NO.168) |
| (Gly⁸,Arg^{25,26,34},β-Ala³⁵,Lys³⁶(N^{ε}-hexadecanoyl))hGLP-1(7-36)NH₂; | (SEQ ID NO.169) |
| (Gly⁸,Arg^{25,26,34},β-Ala³⁵,Lys³⁶(N^{ε}-decanoyl))hGLP-1(7-36)NH₂; | (SEQ ID NO.170) |
| (Gly⁸,Aib³⁵,Lys²⁶(N^{ε}-octanoyl),A6c³²,Arg³⁴)hGLP-1(7-36)NH₂; | (SEQ ID NO.171) |
| (Gly⁸,Aib³⁵,Lys²⁶(N^{ε}-tetradecanoyl),A6c³²,Arg³⁴) | (SEQ ID NO.172) |
| hGLP-1(7-36)NH₂; | |
| (Gly⁸,Aib³⁵,Lys²⁶(N^{ε}-hexadecanoyl),A6c³²,Arg³⁴) | (SEQ ID NO.173) |
| hGLP-1(7-36)NH₂; | |
| (Gly⁸,Aib³⁵,A6c³²,Lys³⁴(N^{ε}-octanoyl))hGLP-1(7-36)NH₂; | (SEQ ID NO.174) |
| (Gly⁸,Aib³⁵,A6c³²,Lys³⁴(N^{ε}-tetradecanoyl))hGLP-1(7-36)NH₂; | (SEQ ID NO.175) |
| (Gly⁸,Aib³⁵,A6c³²,Lys³⁴(N^{ε}-hexadecanoyl))hGLP-1(7-36)NH₂; | (SEQ ID NO.176) |
| (Gly⁸,Aib³⁵,Arg²⁶,A6c³²,Lys³⁴(N^{ε}-octanoyl))hGLP-1(7-36)NH₂; | (SEQ ID NO.177) |
| (Gly⁸,Aib³⁵,Arg²⁶,A6c³²,Lys³⁴(N^{ε}-tetradecanoyl))hGLP-1(7-36)NH₂; | (SEQ ID NO.178) |
| (Gly⁸,Aib³⁵,A6c³²,Lys³⁶(N^{ε}-octanoyl))hGLP-1(7-36)NH₂; | (SEQ ID NO.179) |
| (Gly⁸,Aib³⁵,A6c³²,Lys³⁶(N^{ε}-tetradecanoyl))hGLP-1(7-36)NH₂; | (SEQ ID NO.180) |
| (Gly⁸,Aib³⁵,A6c³²,Lys³⁶(N^{ε}-hexadecanoyl))hGLP-1(7-36)NH₂; | (SEQ ID NO.181) |
| (Gly⁸,Aib³⁵,Arg²⁶,A6c³²,Lys³⁶(N^{ε}-octanoyl))hGLP-1(7-36)NH₂; | (SEQ ID NO.182) |
| (Gly⁸,Aib³⁵,Arg²⁶,A6c³²,Lys³⁶(N^{ε}-tetradecanoyl))hGLP-1(7-36)NH₂; | (SEQ ID NO.183) |
| (Gly⁸,Aib³⁵,Arg²⁶,A6c³²,Lys³⁶(N^{ε}-hexadecanoyl))hGLP-1(7-36)NH₂; | (SEQ ID NO.184) |
| (Gly⁸,Aib³⁵,Arg^{26,34},A6c³²,Lys³⁶(N^{ε}-octanoyl))hGLP-1(7-36)NH₂; | (SEQ ID NO.185) |
| (Gly⁸,Aib³⁵,Arg^{26,34},A6c³²,Lys³⁶(N^{ε}-decanoyl))hGLP-1(7-36)NH₂; | (SEQ ID NO.186) |
| (Gly⁸,Aib³⁵,Arg^{26,34},A6c³²,Lys³⁶(N^{ε}-tetradecanoyl)) | (SEQ ID NO.187) |
| hGLP-1(7-36)NH₂; | |
| (Gly⁸,Aib³⁵,Arg^{26,34},A6c³²,Lys³⁶(N^{ε}-hexadecanoyl)) | (SEQ ID NO.188) |
| hGLP-1(7-36)NH₂; | |
| (Gly⁸,Aib^{24,35},Lys²⁶(N^{ε}-octanoyl),Arg³⁴)hGLP-1(7-36)NH₂; | (SEQ ID NO.189) |
| (Gly⁸,Aib^{24,35},Lys²⁶(N^{ε}-tetradecanoyl),Arg³⁴)hGLP-1(7-36)NH₂; | (SEQ ID NO.190) |
| (Gly⁸,Aib^{24,35},Lys²⁶(N^{ε}-hexadecanoyl),Arg³⁴)hGLP-1(7-36)NH₂; | (SEQ ID NO.191) |
| (Gly⁸,Aib^{24,35},Arg²⁶,Lys³⁴(N^{ε}-octanoyl))hGLP-1(7-36)NH₂; | (SEQ ID NO.192) |
| (Gly⁸,Aib^{24,35},Arg²⁶,Lys³⁴(N^{ε}-tetradecanoyl))hGLP-1(7-36)NH₂; | (SEQ ID NO.193) |
| (Gly⁸,Aib^{24,35},Arg²⁶,Lys³⁴(N^{ε}-hexadecanoyl))hGLP-1(7-36)NH₂; | (SEQ ID NO.194) |
| (Gly⁸,Aib^{24,35},Arg^{26,34},Lys³⁶(N^{ε}-octanoyl))hGLP-1(7-36)NH₂; | (SEQ ID NO.195) |
| (Gly⁸,Aib^{24,35},Arg^{26,34},Lys³⁶(N^{ε}-tetradecanoyl))hGLP-1(7-36)NH₂; | (SEQ ID NO.196) |
| (Gly⁸,Aib^{24,35},Arg^{26,34},Lys³⁶(N^{ε}-hexadecanoyl))hGLP-1(7-36)NH₂; | (SEQ ID NO.197) |
| (Gly⁸,Aib^{24,35},Glu²³,A6c³²,Lys³⁴(N^{e}-octanoyl))hGLP-1(7-36)NH₂; | (SEQ ID NO.198) |
| (Gly⁸,Aib³⁵,Glu²³,Lys²⁶(N^{ε}-octanoyl),Arg³⁴)hGLP-1(7-36)NH₂; | (SEQ ID NO.199) |
| (Gly⁸,Aib³⁵,Glu²³,Lys²⁶(N^{ε}-tetradecanoyl),Arg³⁴)hGLP-1(7-36)NH₂; | (SEQ ID NO.200) |
| (Gly⁸,Aib³⁵,Glu²³,Lys²⁶(N^{ε}-hexadecanoyl),Arg³⁴)hGLP-1(7-36)NH₂; | (SEQ ID NO.201) |
| (Gly⁸,Aib³⁵,Glu²³,Lys³⁴(N^{e}-octanoyl))hGLP-1(7-36)NH₂; | (SEQ ID NO.202) |
| (Gly⁸,Aib³⁵,Glu²³,A6c³²,Lys³⁴(N^{e}-octanoyl))hGLP-1(7-36)NH₂; | (SEQ ID NO.203) |
| (Gly⁸,Aib³⁵,Glu²³,Arg²⁶,Lys³⁴(N^{ε}-octanoyl))hGLP-1(7-36)NH₂; | (SEQ ID NO.204) |
| (Gly⁸,Aib³⁵,Glu²³,Arg²⁶,Lys³⁴(N^{ε}-tetradecanoyl))hGLP-1(7-36)NH₂; | (SEQ ID NO.205) |
| (Gly⁸,Aib³⁵,Glu²³,Arg²⁶,Lys³⁴(N^{ε}-hexadecanoyl))hGLP-1(7-36)NH₂; | (SEQ ID NO.206) |
| (Gly⁸,Aib³⁵,Glu²³,Lys³⁶(N^{ε}-octanoyl))hGLP-1(7-36)NH₂; | (SEQ ID NO.207) |
| (Gly⁸,Aib³⁵,Glu²³,Lys³⁶(N^{ε}-tetradecanoyl))hGLP-1(7-36)NH₂; | (SEQ ID NO.208) |
| (Gly⁸,Aib³⁵,Glu²³,Lys³⁶(N^{ε}-hexadecanoyl))hGLP-1(7-36)NH₂; | (SEQ ID NO.209) |
| (Gly⁸,Aib³⁵,Glu²³,Arg^{26,34},Lys³⁶(N^{ε}-octanoyl))hGLP-1(7-36)NH₂; | (SEQ ID NO.210) |
| (Gly⁸,Aib³⁵,Glu²³,Arg^{26,34},Lys³⁶(N^{ε}-tetradecanoyl)) | (SEQ ID NO.211) |
| hGLP-1(7-36)NH₂; | |
| (Gly⁸,Aib³⁵,Glu²³,Arg^{26,34},Lys³⁶(N^{ε}-hexadecanoyl)) | (SEQ ID NO.212) |
| hGLP-1(7-36)NH₂; | |
| (Gly⁸,Aib^{30,35},Lys²⁶(N^{ε}-octanoyl),Arg³⁴)hGLP-1(7-36)NH₂; | (SEQ ID NO.213) |
| (Gly⁸,Aib^{30,35},Lys²⁶(N^{ε}-tetradecanoyl),Arg³⁴)hGLP-1(7-36)NH₂; | (SEQ ID NO.214) |
| (Gly⁸,Aib^{30,35},Lys²⁶(N^{ε}-hexadecanoyl),Arg³⁴)hGLP-1(7-36)NH₂; | (SEQ ID NO.215) |
| (Gly⁸,Aib^{30,35},Arg²⁶,Lys³⁴(N^{ε}-octanoyl))hGLP-1(7-36)NH₂; | (SEQ ID NO.216) |
| (Gly⁸,Aib^{30,35},Arg²⁶,Lys³⁴(N^{ε}-tetradecanoyl))hGLP-1(7-36)NH₂; | (SEQ ID NO.217) |
| (Gly⁸,Aib^{30,35},Arg²⁶,Lys³⁴(N^{ε}-hexadecanoyl))hGLP-1(7-36)NH₂; | (SEQ ID NO.218) |
| (Gly⁸,Aib^{30,35},Arg^{26,34},Lys³⁶(N^{ε}-octanoyl))hGLP-1(7-36)NH₂; | (SEQ ID NO.219) |
| (Gly⁸,Aib^{30,35},Arg^{26,34},Lys³⁶(N^{ε}-tetradecanoyl))hGLP-1(7-36)NH₂; | (SEQ ID NO.220) |
| (Gly⁸,Aib^{30,35},Arg^{26,34},Lys³⁶(N^{ε}-hexadecanoyl))hGLP-1(7-36)NH₂; | (SEQ ID NO.221) |
| (Gly⁸,Aib³⁵,Glu²³,A6c³²,Lys³⁶(N^{ε}-octanoyl))hGLP-1(7-36)NH₂; | (SEQ ID NO.222) |
| (Gly⁸,Aib³⁵,Glu²³,A6c³²,Lys³⁶(N^{ε}-tetradecanoyl))hGLP-1(7-36)NH₂; | (SEQ ID NO.223) |
| (Gly⁸,Aib³⁵,Glu²³,A6c³²,Lys³⁶(N^{ε}-hexadecanoyl))hGLP-1(7-36)NH₂; | (SEQ ID NO.224) |
| (Gly⁸,Aib³⁵,Glu²³,Arg^{26,34},A6c³²,Lys³⁶(N^{ε}-octanoyl)) | (SEQ ID NO.225) |
| hGLP-1(7-36)NH₂; | |
| (Gly⁸,Aib³⁵,Glu²³,Arg^{26,34},A6c³²,Lys³⁶(N^{ε}-tetradecanoyl)) | (SEQ ID NO.226) |
| hGLP-1(7-36)NH₂; | |
| (Gly⁸,Aib³⁵,Glu²³,Arg^{26,34},A6c³²,Lys³⁶(N^{ε}-hexadecanoyl)) | (SEQ ID NO.227) |
| hGLP-1(7-36)NH₂; | |
| (Gly⁸,Aib^{24,35},Glu²³,Arg^{26,34},A6c³²,Lys³⁶(N^{ε}-octanoyl)) | (SEQ ID NO.228) |
| hGLP-1(7-36)NH₂; | |
| (Gly⁸,Aib²⁴,34,Glu²³,Arg^{26,34},A6c³²,Lys³⁶(N^{ε}-tetradecanoyl)) | (SEQ ID NO.229) |
| hGLP-1(7-36)NH₂; | |
| (Gly⁸,Aib^{24,35},Glu²³,Arg^{26,34},A6c³²,Lys³⁶(N^{ε}-hexadecanoyl)) | (SEQ ID NO.230) |
| hGLP-1(7-36)NH₂; | |
| (Gly⁸,Aib^{24,30,35},Glu²³,Arg^{26,34},A6c³²,Lys³⁶(N^{ε}-octanoyl)) | (SEQ ID NO.231) |
| hGLP-1(7-36)NH₂; | |
| (Gly⁸,Aib^{24,30,35},Glu²³,Arg^{26,34},A6c³²,Lys³⁶(N^{ε}-tetradecanoyl)) | (SEQ ID NO.232) |
| hGLP-1(7-36)NH₂; | |
| (Gly⁸,Aib^{24,30,35},Glu²³,Arg^{26,34},A6c³²,Lys³⁶(N^{ε}-hexadecanoyl)) | (SEQ ID NO.233) |
| hGLP-1(7-36)NH₂; | |
| ((N^{α}-HEPES-His)⁷,Gly⁸,β-Ala³⁵)hGLP-1(7-36)NH₂; | (SEQ ID NO.234) |
| ((N^{α}-HEPA-His)⁷,Gly⁸,β-Ala³⁵)hGLP-1(7-36)NH₂; | (SEQ ID NO.235) |
| ((N^{α}-tetradecanoyl-His)⁷,Gly⁸,Aib³⁵)hGLP-1(7-36)NH₂; | (SEQ ID NO.236) |
| ((N^{α}-tetradecanoyl-His)⁷,Gly⁸,β-Ala³⁵)hGLP-1(7-36)NH₂; | (SEQ ID NO.237) |
| ((N^{α}-tetradecanoyl-His)⁷,Gly⁸,Aib³⁵,Arg^{26,34}) | (SEQ ID NO.238) |
| hGLP-1(7-36)NH₂; | |
| ((N^{α}-tetradecanoyl-His)⁷,Gly⁸,Arg^{26,34},β-Ala³⁵) | (SEQ ID NO.239) |
| hGLP-1(7-36)NH₂; | |
| ((N^{α}-tetradecanoyl-His)⁷,Gly⁸,Aib³⁵,Arg^{25,26,34}) | (SEQ ID NO.240) |
| hGLP-1(7-36)NH₂; | |
| ((N^{α}-tetradecanoyl-His)⁷,Gly⁸,Arg^{25,26,34},β-Ala³⁵) | (SEQ ID NO.241) |
| hGLP-1(7-36)NH₂; | |
| (Gly⁸,Aib³⁵,Lys²⁶(N^{ε}-octanesulfonyl),Arg³⁴) | (SEQ ID NO.242) |
| hGLP-1(7-36)NH₂; | |
| (Gly⁸,Aib³⁵,Lys²⁶(N^{ε}-dodecanesulfonyl),Arg³⁴) | (SEQ ID NO.243) |
| hGLP-1(7-36)NH₂; | |
| (Gly⁸,Aib³⁵,Lys²⁶(N^{ε}-hexadecanesulfonyl),Arg³⁴) | (SEQ ID NO.244) |
| hGLP-1(7-36)NH₂; | |
| (Gly⁸,Aib³⁵,Arg²⁶,Lys³⁴(N^{ε}-octanesulfonyl)) | (SEQ ID NO.245) |
| hGLP-1(7-36)NH₂; | |
| (Gly⁸,Aib³⁵,Arg²⁶,Lys³⁴(N^{ε}-dodecanesulfonyl)) | (SEQ ID NO.246) |
| hGLP-1(7-36)NH₂; | |
| (Gly⁸,Aib³⁵,Arg²⁶,Lys³⁴(N^{ε}-hexadecanesulfonyl)) | (SEQ ID NO.247) |
| hGLP-1(7-36)NH₂; | |
| (Gly⁸,Aib³⁵,Arg^{26,34},Lys³⁶(N^{ε}-octanesulfonyl)) | (SEQ ID NO.248) |
| hGLP-1(7-36)NH₂; | |
| (Gly⁸,Aib³⁵,Arg^{26,34},Lys³⁶(N^{ε}-hexadecanesulfonyl)) | (SEQ ID NO.249) |
| hGLP-1(7-36)NH₂; | |
| (Gly⁸,Aib³⁵,Asp²⁶(1-(4-decylpiperazine)),Arg³⁴) | (SEQ ID NO.250) |
| hGLP-1(7-36)NH₂; | |
| (Gly⁸,Aib³⁵,Asp²⁶(1-(4-dodecylpiperazine)),Arg³⁴) | (SEQ ID NO.251) |
| hGLP-1(7-36)NH₂; | |
| (Gly⁸,Aib³⁵,Asp²⁶(1-(4-tetradecylpiperazine)),Arg³⁴) | (SEQ ID NO.252) |
| hGLP-1(7-36)NH₂; | |
| (Gly⁸,Aib³⁵,Asp²⁶(1-(4-hexadecylpiperazine)),Arg³⁴) | (SEQ ID NO.253) |
| hGLP-1(7-36)NH₂; | |
| (Gly⁸,Aib³⁵,Arg²⁶,Asp³⁴(1-(4-decylpiperazine))) | (SEQ ID NO.254) |
| hGLP-1(7-36)NH₂; | |
| (Gly⁸,Aib³⁵,Arg²⁶,Asp³⁴(1-(4-dodecylpiperazine))) | (SEQ ID NO.255) |
| hGLP-1(7-36)NH₂; | |
| (Gly⁸,Aib³⁵,Arg²⁶,Asp³⁴(1-(4-tetradecylpiperazine))) | (SEQ ID NO.256) |
| hGLP-1(7-36)NH₂; | |
| (Gly⁸,Aib³⁵,Arg²⁶,Asp³⁴(1-(4-hexadecylpiperazine))) | (SEQ ID NO.257) |
| hGLP-1(7-36)NH₂; | |
| (Gly⁸,Aib³⁵,Arg^{26,34},Asp³⁶(1-(4-decylpiperazine))) | (SEQ ID NO.258) |
| hGLP-1(7-36)NH₂; | |
| (Gly⁸,Aib³⁵,Arg^{26,34},Asp³⁶(1-(4-dodecylpiperazine))) | (SEQ ID NO.259) |
| hGLP-1(7-36)NH₂; | |
| (Gly⁸,Aib³⁵,Arg^{26,34},Asp³⁶(1-(4-hexadecylpiperazine))) | (SEQ ID NO.260) |
| hGLP-1(7-36)NH₂; | |
| (Gly⁸,Aib³⁵,Arg^{26,34},Asp³⁸(1-(4-decylpiperazine))) | (SEQ ID NO.261) |
| hGLP-1(7-38)NH₂; | |
| (Gly⁸,Aib³⁵,Arg^{26,34},Asp³⁸(1-(4-dodecylpiperazine))) | (SEQ ID NO.262) |
| hGLP-1(7-38)NH₂; | |
| (Gly⁸,Aib³⁵,Arg^{26,34},Asp³⁸(1-(4-tetradecylpiperazine))) | (SEQ ID NO.263) |
| hGLP-1(7-38)NH₂; | |
| (Gly⁸,Aib³⁵,Arg^{26,34},Asp³⁸(1-(4-hexadecylpiperazine))) | (SEQ ID NO.264) |
| hGLP-1(7-38)NH₂; | |
| (Gly⁸,Aib^{35,37},Arg^{26,34},Asp³⁸(1-(4-decylpiperazine))) | (SEQ ID NO.265) |
| hGLP-1(7-38)NH₂; | |
| (Gly⁸,Aib^{35,37},Arg^{26,34},Asp³⁸(1-(4-dodecylpiperazine))) | (SEQ ID NO.266) |
| hGLP-1(7-38)NH₂; | |
| (Gly⁸,Aib^{35,37},Arg^{26,34},Asp³⁸(1-(4-tetradecylpiperazine))) | (SEQ ID NO.267) |
| hGLP-1(7-38)NH₂; | |
| (Gly⁸,Aib^{35,37},Arg^{26,34},Asp³⁸(1-(4-hexadecylpiperazine))) | (SEQ ID NO.268) |
| hGLP-1(7-38)NH₂; | |
| (Gly⁸,Aib³⁵,Arg^{25,34},Asp²⁶(1-(4-decylpiperazine))) | (SEQ ID NO.269) |
| hGLP-1(7-36)NH₂; | |
| (Gly⁸,Aib³⁵,Arg^{25,34},Asp²⁶(1-(4-dodecylpiperazine))) | (SEQ ID NO.270) |
| hGLP-1(7-36)NH₂; | |
| (Gly⁸,Aib³⁵,Arg^{25,34},Asp²⁶(1-(4-tetradecylpiperazine))) | (SEQ ID NO.271) |
| hGLP-1(7-36)NH₂; | |
| (Gly⁸,Aib³⁵,Arg^{25,34},Asp²⁶(1-(4-hexadecylpiperazine))) | (SEQ ID NO.272) |
| hGLP-1(7-36)NH₂; | |
| (Gly⁸,Aib³⁵,Arg^{25,26},Asp³⁴(1-(4-decylpiperazine))) | (SEQ ID NO.273) |
| hGLP-1(7-36)NH₂; | |
| (Gly⁸,Aib³⁵,Arg^{25,26},Asp³⁴(1-(4-dodecylpiperazine))) | (SEQ ID NO.274) |
| hGLP-1(7-36)NH₂; | |
| (Gly⁸,Aib³⁵,Arg^{25,26},Asp³⁴(1-(4-tetradecylpiperazine))) | (SEQ ID NO.275) |
| hGLP-1(7-36)NH₂; | |
| (Gly⁸,Aib³⁵,Arg^{25,26},Asp³⁴(1-(4-hexadecylpiperazine))) | (SEQ ID NO.276) |
| hGLP-1(7-36)NH₂; | |
| (Gly⁸,Aib³⁵,Arg^{25,26,34},Asp³⁶(1-(4-decylpiperazine))) | (SEQ ID NO.277) |
| hGLP-1(7-36)NH₂; | |
| (Gly⁸,Aib³⁵,Arg^{25,26,34},Asp³⁶(1-(4-dodecylpiperazine))) | (SEQ ID NO.278) |
| hGLP-1(7-36)NH₂; | |
| (Gly⁸,Aib³⁵,Arg^{25,26,34},Asp³⁶(1-(4-tetradecylpiperazine))) | (SEQ ID NO.279) |
| hGLP-1(7-36)NH₂; | |
| (Gly⁸,Aib³⁵,Arg^{25,26,34},Asp³⁶(1-(4-hexadecylpiperazine))) | (SEQ ID NO.280) |
| hGLP-1(7-36)NH₂; | |
| (Gly⁸,Aib³⁵,Arg^{25,26,34},Asp³⁸(1-(4-decylpiperazine))) | (SEQ ID NO.281) |
| hGLP-1(7-38)NH₂; | |
| (Gly⁸,Aib³⁵,Arg^{25,26,34},Asp³⁸(1-(4-dodecylpiperazine))) | (SEQ ID NO.282) |
| hGLP-1(7-38)NH₂; | |
| (Gly⁸,Aib³⁵,Arg^{25,26,34},Asp³⁸(1-(4-tetradecylpiperazine))) | (SEQ ID NO.283) |
| hGLP-1(7-38)NH₂; | |
| (Gly⁸,Aib³⁵,Arg^{25,26,34},Asp³⁸(1-(4-hexadecylpiperazine))) | (SEQ ID NO.284) |
| hGLP-1(7-38)NH₂; | |
| (Gly⁸,Aib^{35,37},Arg^{25,26,34},Asp³⁸(1-(4-decylpiperazine))) | (SEQ ID NO.285) |
| hGLP-1(7-38)NH₂; | |
| (Gly⁸,Aib^{35,37},Arg^{25,26,34},Asp³⁸(1-(4-dodecylpiperazine))) | (SEQ ID NO.286) |
| hGLP-1(7-38)NH₂; | |
| (Gly⁸,Aib^{35,37},Arg^{25,26,34},Asp³⁸(1-(4-tetradecylpiperazine))) | (SEQ ID NO.287) |
| hGLP-1(7-38)NH₂; | |
| (Gly⁸,Aib^{35,37},Arg^{25,26,34},Asp³⁸(1-(4-hexadecylpiperazine))) | (SEQ ID NO.288) |
| hGLP-1(7-38)NH₂; | |
| Gly⁸,Aib³⁵,Arg^{26,34},Glu³⁶(1-dodecylamino)) | (SEQ ID NO.289) |
| hGLP-1(7-36)NH₂; | |
| (Gly⁸,Aib³⁵,Glu²⁶(1-dodecylamino),Arg³⁴) | (SEQ ID NO.290) |
| hGLP-1(7-36)NH₂; | |
| (Gly⁸,Aib³⁵,Arg²⁶,Glu³⁴(1-dodecylamino)) | (SEQ ID NO.291) |
| hGLP-1(7-36)NH₂; | |
| (Gly⁸,Aib^{35,37},Arg^{26,34},Glu³⁸(1-dodecylamino)) | (SEQ ID NO.292) |
| hGLP-1(7-38)NH₂; | |
| (Gly⁸,Aib³⁵,Arg³⁴,Lys²⁶(N^{ε}-(2-(4-decyl-1-piperazine)-acetyl))) | (SEQ ID NO.293) |
| hGLP-1(7-36)NH₂; | |
| (Gly⁸,Aib³⁵,Arg³⁴,Lys²⁶(N^{ε}-(2-(4-dodecyl-1-piperazine)-acetyl))) | (SEQ ID NO.294) |
| hGLP-1(7-36)NH₂; | |
| (Gly⁸,Aib³⁵,Arg³⁴,Lys²⁶(N^{ε}-(2-(4-tetradecyl-1-piperazine)-acetyl))) | (SEQ ID NO.295) |
| hGLP-1(7-36)NH₂; | |
| (Gly⁸,Aib³⁵,Arg³⁴,Lys²⁶(N^{ε}-(2-(4-hexadecyl-1-piperazine)-acetyl))) | (SEQ ID NO.296) |
| hGLP-1(7-36)NH₂; | |
| (Gly⁸,Aib³⁵,Arg²⁶,Lys³⁴(N^{ε}-(2-(4-decyl-1-piperazine)-acetyl))) | (SEQ ID NO.297) |
| hGLP-1(7-36)NH₂; | |
| (Gly⁸,Aib³⁵,Arg²⁶,Lys³⁴(N^{ε}-(2-(4-dodecyl-1-piperazine)-acetyl)) | (SEQ ID NO.298) |
| hGLP-1(7-36)NH₂; | |
| (Gly⁸,Aib³⁵,Arg²⁶,Lys³⁴(N^{ε}-(2-(4-tetradecyl-1-piperazine)-acetyl))) | (SEQ ID NO.299) |
| hGLP-1(7-36)NH₂; | |
| (Gly⁸,Aib³⁵,Arg²⁶,Lys³⁴(N^{ε}-(2-(4-hexadecyl-1-piperazine)-acetyl))) | (SEQ ID NO.300) |
| hGLP-1(7-36)NH₂; | |
| (Gly⁸,Aib³⁵,Arg^{26,34},Lys³⁶(N^{ε}-(2-(4-decyl-1-piperazine)-acetyl))) | (SEQ ID NO.301) |
| hGLP-1(7-36)NH₂; | |
| (Gly⁸,Aib³⁵,Arg^{26,34},Lys³⁶(N^{ε}-(2-(4-dodecyl-1-piperazine)-acetyl))) | (SEQ ID NO.302) |
| hGLP-1(7-36)NH₂; | |
| (Gly⁸,Aib³⁵,Arg^{26,34},Lys³⁶(N^{ε}-(2-(4-hexadecyl-1-piperazine)-acetyl))) | (SEQ ID NO.303) |
| hGLP-1(7-36)NH₂; | |
| (Gly⁸,Aib³⁵,Arg^{26,34},Lys³⁸(N^{ε}-(2-(4-decyl-1-piperazine)-acetyl))) | (SEQ ID NO.304) |
| hGLP-1(7-38)NH₂; | |
| (Gly⁸,Aib³⁵,Arg^{26,34},Lys³⁸(N^{ε}-(2-(4-dodecyl-1-piperazine)-acetyl))) | (SEQ ID NO.305) |
| hGLP-1(7-38)NH₂; | |
| (Gly⁸,Aib³⁵,Arg^{26,34},Lys³⁸(N^{ε}-(2-(4-tetradecyl-1-piperazine)-acetyl))) | (SEQ ID NO.306) |
| hGLP-1(7-38)NH₂; | |
| (Gly⁸,Aib³⁵,Arg^{26,34},Lys³⁸(N^{ε}-(2-(4-hexadecyl-1-piperazine)-acetyl))) | (SEQ ID NO.307) |
| hGLP-1(7-38)NH₂; | |
| (Gly⁸,Aib^{35,37},Arg^{26,34},Lys³⁸(N^{ε}-(2-(4-decyl-1-piperazine)-acetyl))) | (SEQ ID NO.308) |
| hGLP-1(7-38)NH₂; | |
| (Gly⁸,Aib^{35,37},Arg^{26,34},Lys³⁸(N^{ε}-(2-(4-dodecyl-1-piperazine)-acetyl))) | (SEQ ID NO.309) |
| hGLP-1(7-38)NH₂; | |
| (Gly⁸,Aib^{35,37},Arg^{26,34},Lys³⁸(N^{ε}-(2-(4-tetradecyl-1-piperazine)-acetyl))) | (SEQ ID NO.310) |
| hGLP-1(7-38)NH₂; | |
| (Gly⁸,Aib^{35,37},Arg^{26,34},Lys³⁸(N^{ε}-(2-(4-hexadecyl-1-piperazine)-acetyl))) | (SEQ ID NO.311) |
| hGLP-1(7-38)NH₂; | |
| (Gly⁸,Aib³⁵,Arg^{25,34},Lys²⁶(N^{ε}-(2-(4-decyl-1-piperazine)-acetyl))) | (SEQ ID NO.312) |
| hGLP-1(7-36)NH₂; | |
| (Gly⁸,Aib³⁵,Arg^{25,34},Lys²⁶(N^{ε}-(2-(4-dodecyl-1-piperazine)-acetyl))) | (SEQ ID NO.313) |
| hGLP-1(7-36)NH₂; | |
| (Gly⁸,Aib³⁵,Arg^{25,34},Lys²⁶(N^{ε}-(2-(4-tetradecyl-1-piperazine)-acetyl))) | (SEQ ID NO.314) |
| hGLP-1(7-36)NH₂; | |
| (Gly⁸,Aib³⁵,Arg^{25,34},Lys²⁶(N^{ε}-(2-(4-hexadecyl-1-piperazine)-acetyl))) | (SEQ ID NO.315) |
| hGLP-1(7-36)NH₂; | |
| (Gly⁸,Aib³⁵,Arg^{25,26},Lys³⁴(N^{ε}-(2-(4-decyl-1-piperazine)-acetyl))) | (SEQ ID NO.316) |
| hGLP-1(7-36)NH₂; | |
| (Gly⁸,Aib³⁵,Arg^{25,26},Lys³⁴(N^{ε}-(2-(4-dodecyl-1-piperazine)-acetyl))) | (SEQ ID NO.317) |
| hGLP-1(7-36)NH₂; | |
| (Gly⁸,Aib³⁵,Arg^{25,26},Lys³⁴(N^{ε}-(2-(4-tetradecyl-1-piperazine)-acetyl))) | (SEQ ID NO.318) |
| hGLP-1(7-36)NH₂; | |
| (Gly⁸,Aib³⁵,Arg^{25,26},Lys³⁴(N^{ε}-(2-(4-hexadecyl-1-piperazine)-acetyl))) | (SEQ ID NO.319) |
| hGLP-1(7-36)NH₂; | |
| (Gly⁸,Aib³⁵,Arg^{25,26,34},Lys³⁶(N^{ε}-(2-(4-decyl-1-piperazine)-acetyl))) | (SEQ ID NO.320) |
| hGLP-1(7-36)NH₂; | |
| (Gly⁸,Aib³⁵,Arg2526,34,Lys³⁶(N^{ε}-(2-(4-dodecyl-1-piperazine)-acetyl))) | (SEQ ID NO.321) |
| hGLP-1(7-36)NH₂; | |
| (Gly⁸,Aib³⁵,Arg^{25,26,34},Lys³⁶(N^{ε}-(2-(4-tetradecyl-1-piperazine)-acetyl))) | (SEQ ID NO.322) |
| hGLP-1(7-36)NH₂; | |
| (Gly⁸,Aib³⁵,Arg^{25,26,34},Lys³⁶(N^{ε}-(2-(4-hexadecyl-1-piperazine)-acetyl))) | (SEQ ID NO.323) |
| hGLP-1(7-36)NH₂; | |
| (Gly⁸,Aib³⁵,Arg^{25,26,34},Lys³⁸(N^{ε}-(2-(4-decyl-1-piperazine)-acetyl))) | (SEQ ID NO.324) |
| hGLP-1(7-38)NH₂; | |
| (Gly⁸,Aib³⁵,Arg^{25,26,34},Lys³⁸(N^{ε}-(2-(4-dodecyl-1-piperazine)-acetyl))) | (SEQ ID NO.325) |
| hGLP-1(7-38)NH₂; | |
| (Gly⁸,Aib³⁵,Arg^{25,26,34},Lys³⁸(N^{ε}-(2-(4-tetradecyl-1-piperazine)-acetyl))) | (SEQ ID NO.326) |
| hGLP-1(7-38)NH₂; | |
| (Gly⁸,Aib³⁵,Arg^{25,26,34},Lys³⁸(N^{ε}-(2-(4-hexadecyl-1-piperazine)-acetyl))) | (SEQ ID NO.327) |
| hGLP-1(7-38)NH₂; | |
| (Gly⁸,Aib^{35,37},Arg^{25,26,34},Lys³⁸(N^{ε}-(2-(4-decyl-1-piperazine)-acetyl))) | (SEQ ID NO.328) |
| hGLP-1(7-38)NH₂; | |
| (Gly⁸,Aib^{35,37},Arg^{25,26,34},Lys³⁸(N^{ε}-(2-(4-dodecyl-1-piperazine)-acetyl))) | (SEQ ID NO.329) |
| hGLP-1(7-38)NH₂; | |
| (Gly⁸,Aib^{35,37},Arg^{25,26,34},Lys³⁸(N^{ε}-(2-(4-tetradecyl-1-piperazine)-acetyl))) | (SEQ ID NO.330) |
| hGLP-1(7-38)NH₂; | |
| (Gly⁸,Aib^{35,37},Arg^{25,26,34},Lys³⁸(N^{ε}-(2-(4-hexadecyl-1-piperazine)-acetyl))) | (SEQ ID NO.331) |
| hGLP-1(7-38)NH₂; | |
| (Gly⁸,Aib³⁵,Arg^{26,34},Lys³⁶(N^{ε}-decanoyl))hGLP-1(7-36)OH; | (SEQ ID NO.332) |
| (Gly⁸,Aib³⁵,Lys²⁵,Arg^{26,34},Lys³⁶(N^{ε}-decanoyl))hGLP-1(7-36)OH; | (SEQ ID NO.333) |
| (Gly⁸,Aib³⁵,Arg^{26,34},Ava³⁷,Ado³⁸)hGLP-1(7-38)NH₂; | (SEQ ID NO.334) |
| (Gly⁸,Aib³⁵,Arg^{26,34},Asp³⁷,Ava³⁸,Ado³⁹)hGLP-1(7-39)NH₂; | (SEQ ID NO.335) |
| (Gly⁸,Aib³⁵,Arg^{26,34},Aun³⁷)hGLP-1(7-37)NH₂; | (SEQ ID NO.336) |
| (Gly⁸,Aib^{17,35})hGLP-1(7-36)NH₂; | (SEQ ID NO.337) |
| (Gly⁸,Arg^{26,34},β-Ala³⁵,D-Asp³⁷,Ava³⁸,Aun³⁹)hGLP-1(7-39)NH₂; | (SEQ ID NO.338) |
| (Gly⁸,Glu^{22,23},β-Ala³⁵)hGLP-1(7-36)NH₂; | (SEQ ID NO.339) |
| (Gly⁸,Lys¹⁸,β-Ala³⁵)hGLP-1(7-36)NH₂; | (SEQ ID NO.340) |
| (Gly⁸,Leu²⁷,β-Ala³⁵)hGLP-1(7-36)NH₂; | (SEQ ID NO.341) |
| (Gly⁸,Lys³³,β-Ala³⁵)hGLP-1(7-36)NH₂; | (SEQ ID NO.342) |
| (Gly⁸,Lys¹⁸,Leu²⁷,β-Ala³⁵)hGLP-1(7-36)NH₂; | (SEQ ID NO.343) |
| (Gly⁸,D-Arg³⁶)hGLP-1(7-36)NH₂; | (SEQ ID NO.344) |
| (Gly⁸,β-Ala³⁵,D-Arg³⁷)hGLP-1(7-37)NH₂; | (SEQ ID NO.345) |
| (Gly⁸,Aib²⁷,β-Ala³⁵)hGLP-1(7-36)NH₂; | (SEQ ID NO.346) |
| (Gly⁸,Aib²⁷,β-Ala^{35,37},Arg³⁸)hGLP-1(7-38)NH₂; | (SEQ ID NO.347) |
| (Gly⁸,Aib²⁷,β-Ala^{35,37},Arg^{38,39})hGLP-1(7-39)NH₂; | (SEQ ID NO.348) |
| (Gly⁸,Lys¹⁸,27,β-Ala³⁵)hGLP-1(7-36)NH₂; | (SEQ ID NO.349) |
| (Gly⁸,Lys²⁷,β-Ala³⁵)hGLP-1(7-36)NH₂; | (SEQ ID NO.350) |
| (Gly⁸,β-Ala³⁵,Arg³⁸)hGLP-1(7-38)NH₂; | (SEQ ID NO.351) |
| (Gly⁸,Arg^{26,34},β-Ala³⁵)hGLP-1(7-36)NH₂; | (SEQ ID NO.352) |
| (Gly⁸,D-Arg³⁵)hGLP-1(7-36)NH₂; | (SEQ ID NO.353) |
| (Gly⁸,β-Ala³⁵,Arg³⁷)hGLP-1(7-37)NH₂; | (SEQ ID NO.354) |
| (Gly⁸,Phe³¹,β-Ala³⁵)hGLP-1(7-36)NH₂; | (SEQ ID NO.355) |
| (Gly⁸,Aib³⁵,Phe³¹)hGLP-1(7-36)NH₂; | (SEQ ID NO.356) |
| (Gly⁸,Aib³⁵,2-Nal³¹)hGLP-1(7-36)NH₂; | (SEQ ID NO.357) |
| (Gly⁸,Aib³⁵,2-Nal^{28,31})hGLP-1(7-36)NH₂; | (SEQ ID NO.358) |
| (Gly⁸,Aib³⁵,Arg^{26,34},2-Nal³¹)hGLP-1(7-36)NH₂; | (SEQ ID NO.359) |
| (Gly⁸,Aib³⁵,Arg^{26,34},Phe³¹)hGLP-1(7-36)NH₂; | (SEQ ID NO.360) |
| (Gly⁸,Aib³⁵,2-Nal^{19,31})hGLP-1(7-36)NH₂; | (SEQ ID NO.361) |
| (Gly⁸,Aib³⁵,2-Nal^{12,31})hGLP-1(7-36)NH₂; | (SEQ ID NO.362) |
| (Gly⁸,Aib³⁵,Lys³⁶(N^{ε}-decanoyl))hGLP-1(7-36)NH₂; | (SEQ ID NO.363) |
| (Gly⁸,Aib³⁵,Arg³⁴,Lys²⁶(N^{ε}-decanoyl))hGLP-1(7-36)NH₂; | (SEQ ID NO.364) |
| (Gly⁸,Aib³⁵,Arg^{26,34},Lys³⁶(N^{ε}-dodecanoyl))hGLP-1(7-36)NH₂; | (SEQ ID NO.365) |
| (Gly⁸,β-Ala³⁵,Ser³⁷(O-decanoyl))hGLP1(7-37)-NH₂; | (SEQ ID NO.366) |
| (Gly⁸,Aib²⁷,β-Ala^{35,37},Arg³⁸,Lys³⁹(N^{ε}-octanoyl))hGLP-1(7-39)NH₂; | (SEQ ID NO.367) |
| (Gly⁸,Arg^{26,34},β-Ala³⁵,Lys³⁷(N^{ε}-octanoyl))hGLP-1(7-37)NH₂; | (SEQ ID NO.368) |
| (Gly⁸,Arg^{26,34},β-Ala³⁵,Lys³⁷(N^{ε}-decanoyl))hGLP-1(7-37)NH₂; | (SEQ ID NO.369) |
| (Gly⁸,Arg^{26,34},β-Ala³⁵,Lys³⁷(N^{ε}-tetradecanoyl))hGLP-1(7-37)NH₂; | (SEQ ID NO.370) |
| (Gly⁸,Arg^{26,34},β-Ala³⁵,Lys³⁷(N^{ε}-dodecanoyl))hGLP-1(7-37)NH₂; | (SEQ ID NO.371) |
| (Gly⁸,Arg^{26,34},β-Ala³⁵,Lys³⁷(N^{ε}-dodecanoyl))hGLP-1(8-37)NH₂; | (SEQ ID NO.372) |
| (Gly⁸,Arg²⁶,Aib³⁵)hGLP-1(7-36)NH₂; | (SEQ ID NO.373) |
| (Gly⁸,Arg²⁶,Phe³¹,Aib³⁵)hGLP-1(7-36)NH₂; | (SEQ ID NO.374) |
| (Gly⁸,Arg²⁶,Phe³¹,β-Ala³⁵)hGLP-1(7-36)NH₂; | (SEQ ID NO.375) |
| (Gly⁸,Arg²⁶,β-Ala³⁵)hGLP-1(7-36)NH₂; | (SEQ ID NO.376) |
| (Gly⁸,Arg^{26,34},Phe³¹,Aib³⁵)hGLP-1(7-36)NH₂; | (SEQ ID NO.377) |
| (Gly⁸,Arg^{26,34},Phe³¹,β-Ala³⁵)hGLP-1(7-36)NH₂; | (SEQ ID NO.378) |
| (Gly⁸,Arg³⁴,Aib³⁵)hGLP-1(7-36)NH₂; | (SEQ ID NO.379) |
| (Gly⁸,Arg³⁴,β-Ala³⁵)hGLP-1(7-36)NH₂; | (SEQ ID NO.380) |
| (Gly⁸,Phe³¹,Arg³⁴,Aib³⁵)hGLP-1(7-36)NH₂; | (SEQ ID NO.381) |
| (Gly⁸,Phe³¹,Arg³⁴,β-Ala³⁵)hGLP-1(7-36)NH₂; | (SEQ ID NO.382) |
| (Gly⁸,Arg^{26,34},β-Ala³⁵,Lys³⁶(N^{ε}-Aec-decanoyl))hGLP-1(7-36)NH₂; | (SEQ ID NO.383) |
| or | |
| (Gly⁸,β-Ala³⁵,Ser³⁷(O-decanoyl))hGLP1(7-37)-NH₂; | (SEQ ID NO.384) |

or a pharmaceutically acceptable salt thereof.

12. A compound according to claim 10, wherein said compound is according to the formula:

| | |
|---|---|
| ((N^{α}-Me-His)⁷,Ser⁸,Aib³⁵)hGLP-1(7-36)NH₂; | (SEQ ID NO.385) |
| ((N^{α}-Me-His)⁷,Ser⁸,β-Ala³⁵)hGLP-1(7-36)NH₂; | (SEQ ID NO.386) |
| ((N^{α}-Me-His)⁷,Ser⁸,Aib³⁵,Arg^{26,34})hGLP-1(7-36)NH₂; | (SEQ ID NO.387) |
| ((N^{α}-Me-His)⁷,Ser⁸,Arg^{26,34},β-Ala³⁵)hGLP-1(7-36)NH₂; | (SEQ ID NO.388) |
| (Ser⁸,A6c³⁵)hGLP-1(7-36)NH₂; | (SEQ ID NO.389) |
| (Ser⁸,A5c³⁵)hGLP-1(7-36)NH₂: | (SEQ ID NO.390) |
| (Ser⁸,D-Ala³⁵)hGLP-1(7-36)NH₂; (SEQ ID NO.391) | |
| (Ser⁸,Aib³⁵,A6c³²)hGLP-1(7-36)NH₂; | (SEQ ID NO.392) |
| (Ser⁸,Aib³⁵,A5c³²)hGLP-1(7-36)NH₂; | (SEQ ID NO.393) |
| (Ser⁸,Aib³⁵,Glu²³)hGLP-1(7-36)NH₂; | (SEQ ID NO.394) |
| (Ser⁸,Aib^{24,35})hGLP-1(7-36)NH₂; | (SEQ ID NO.395) |
| (Ser⁸,Aib^{30,35})hGLP-1(7-36)NH₂; | (SEQ ID NO.396) |
| (Ser⁸,Aib^{25,35})hGLP-1(7-36)NH₂; | (SEQ ID NO.397) |
| (Ser⁸,Aib³⁵,A6c^{16,20})hGLP-1(7-36)NH₂; | (SEQ ID NO.398) |
| (Ser⁸,Aib³⁵,A6c^{16,29,32})hGLP-1(7-36)NH₂; | (SEQ ID NO.399) |
| (Ser⁸,Aib³⁵,A6c^{20,32})hGLP-1(7-36)NH₂; | (SEQ ID NO.400) |
| (Ser⁸,Aib³⁵,A6c²⁰)hGLP-1(7-36)NH₂; | (SEQ ID NO.401) |
| (Ser⁸,Aib³⁵,Lys²⁵)hGLP-1(7-36)NH₂; | (SEQ ID NO.402) |
| (Ser⁸,Aib^{24,35},A6c²⁰)hGLP-1(7-36)NH₂; | (SEQ ID NO.403) |
| (Ser⁸,Aib³⁵,A6c^{29,32})hGLP-1(7-36)NH₂; | (SEQ ID NO.404) |
| (Ser⁸,Aib^{24,35},A6c^{29,32})hGLP-1(7-36)NH₂; | (SEQ ID NO.405) |
| (Ser⁸,Aib³⁵,A6c¹²)hGLP-1(7-36)NH₂; | (SEQ ID NO.406) |
| (Ser⁸,Aib³⁵,Cha²⁰)hGLP-1(7-36)NH₂; | (SEQ ID NO.407) |
| (Ser⁸,Aib³⁵,A6c³³)hGLP-1(7-36)NH₂; | (SEQ ID NO.408) |
| (Ser⁸,A6c^{16,20},β-Ala³⁵)hGLP-1(7-36)NH₂; | (SEQ ID NO.409) |
| (Ser⁸,Aib³⁵,β-Ala²²)hGLP-1(7-36)NH₂; | (SEQ ID NO.410) |
| (Ser⁸,Aib^{22,35})hGLP-1(7-36)NH₂; | (SEQ ID NO.411) |
| (Ser⁸,Aib³⁵,Glu²³,A6c³²)hGLP-1(7-36)NH₂; | (SEQ ID NO.412) |
| (Ser⁸,Aib^{24,35},Glu²³,A6c³²)hGLP-1(7-36)NH₂; | (SEQ ID NO.413) |
| (Ser⁸,Aib^{24,25,35},Glu²³,A6c³²)hGLP-1(7-36)NH₂; | (SEQ ID NO.414) |
| (Ser⁸,Aib^{24,25,35},A6c^{16,20,32},Glu²³)hGLP-1(7-36)NH₂; | (SEQ ID NO.415) |
| (Ser⁸,A6c³²,β-Ala³⁵)hGLP-1(7-36)NH₂; | (SEQ ID NO.416) |
| (Ser⁸,A5c³²,β-Ala³⁵)hGLP-1(7-36)NH₂; | (SEQ ID NO.417) |
| (Ser⁸,Glu²³,β-Ala³⁵)hGLP-1(7-36)NH₂; | (SEQ ID NO.418) |
| (Ser⁸,Aib²⁴,β-Ala³⁵)hGLP-1(7-36)NH₂; | (SEQ ID NO.419) |
| (Ser⁸,Aib³⁰,β-Ala³⁵)hGLP-1(7-36)NH₂; | (SEQ ID NO.420) |
| (Ser⁸,Aib²⁵,β-Ala³⁵)hGLP-1(7-36)NH₂; | (SEQ ID NO.421) |
| (Ser⁸,A6c^{16,29,32},β-Ala³⁵)hGLP-1(7-36)NH₂; | (SEQ ID NO.422) |
| (Ser⁸,A6c^{20,32},β-Ala³⁵)hGLP-1(7-36)NH₂; | (SEQ ID NO.423) |
| (Ser⁸,A6c²⁰,β-Ala³⁵)hGLP-1(7-36)NH₂; | (SEQ ID NO.424) |
| (Ser⁸,Lys²⁵,β-Ala³⁵)hGLP-1(7-36)NH₂; | (SEQ ID NO.425) |
| (Ser⁸,Aib²⁴,A6c²⁰,β-Ala³⁵)hGLP-1(7-36)NH₂; | (SEQ ID NO.426) |
| (Ser⁸,A6c^{29,32},β-Ala³⁵)hGLP-1(7-36)NH₂; | (SEQ ID NO.427) |
| (Ser⁸,Aib²⁴,A6c^{29,32},β-Ala³⁵)hGLP-1(7-36)NH₂; | (SEQ ID NO.428) |
| (Ser⁸,A6c¹²,β-Ala³⁵)hGLP-1(7-36)NH₂; | (SEQ ID NO.429) |
| (Ser⁸,Cha²⁰,β-Ala³⁵)hGLP-1(7-36)NH₂; | (SEQ ID NO.430) |
| (Ser⁸,A6c³³,β-Ala³⁵)hGLP-1(7-36)NH₂; | (SEQ ID NO.431) |
| (Ser⁸,β-Ala^{22,35})hGLP-1(7-36)NH₂; | (SEQ ID NO.432) |
| (Ser⁸,Aib²²,β-Ala³⁵)hGLP-1(7-36)NH₂; | (SEQ ID NO.433) |
| (Ser⁸,Glu²³,A6c³²,β-Ala³⁵)hGLP-1(7-36)NH₂; | (SEQ ID NO.434) |
| (Ser⁸,Aib²⁴,Glu²³,A6c³²,β-Ala³⁵)hGLP-1(7-36)NH₂; | (SEQ ID NO.435) |
| (Ser⁸,Aib²⁴,Glu²³,A6c³²,Lys³⁴(N^{e}-octanoyl),β-Ala³⁵) | (SEQ ID NO.436) |
| hGLP-1(7-36)NH₂; | |
| (Ser⁸,Aib^{24,25},Glu²³,A6c³²,β-Ala³⁵)hGLP-1(7-36)NH₂; | (SEQ ID NO.437) |
| (Ser⁸,Aib^{24,25},A6c^{16,20,32},Glu²³,β-Ala³⁵)hGLP-1(7-36)NH₂; | (SEQ ID NO.438) |
| (Ser⁸,Aib³⁵,D-Arg³⁶)hGLP-1(7-36)NH₂; | (SEQ ID NO.439) |
| (Ser⁸,Aib³⁵,D-Lys³⁶)hGLP-1(7-36)NH₂; | (SEQ ID NO.440) |
| (Ser⁸,β-Ala³⁵,D-Arg³⁶)hGLP-1(7-36)NH₂; | (SEQ ID NO.441) |
| (Ser⁸,β-Ala³⁵,D-Lys³⁶)hGLP-1(7-36)NH₂; | (SEQ ID NO.442) |
| (Ser⁸,Aib³⁵,Arg^{26,34})hGLP-1(7-36)NH₂; | (SEQ ID NO.443) |
| (Ser⁸,Arg^{26,34},β-Ala³⁵)hGLP-1(7-36)NH₂; | (SEQ ID NO.444) |
| (Ser⁸,Aib³⁵,Arg^{25,26,34})hGLP-1(7-36)NH₂; | (SEQ ID NO.445) |
| (Ser⁸,Arg^{25,26,34},β-Ala³⁵)hGLP-1(7-36)NH₂; | (SEQ ID NO.446) |
| (Ser⁸,Arg^{26,34},β-Ala³⁵,Lys³⁶(N^{ε}-tetradecanoyl))hGLP-1(7-36)OH; | (SEQ ID NO.447) |
| (Ser⁸,Aib³⁵,Arg^{26,34},Lys³⁶(N^{ε}-tetradecanoyl))hGLP-1(7-37)OH; | (SEQ ID NO.448) |
| (Ser⁸,Aib^{35,37},Arg^{26,34},Lys³⁶(N^{ε}-tetradecanoyl))hGLP-1(7-37)OH; | (SEQ ID NO.449) |
| (Ser⁸,Aib³⁵,Arg^{26,34},Lys³⁸(N^{ε}-tetradecanoyl),D-Ala³⁷) | (SEQ ID NO.450) |
| hGLP-1(7-37)OH; | |
| (Ser⁸,Aib^{35,37},Arg^{26,34},Lys³⁸(N^{ε}-tetradecanoyl))hGLP-1(7-38)OH; | (SEQ ID NO.451) |
| (Ser⁸,Aib³⁵,Arg^{26,34},β-Ala³⁷,Lys³⁸(N^{ε}-tetradecanoyl)) | (SEQ ID NO.452) |
| hGLP-1(7-38)OH; | |
| (Ser⁸,Aib³⁵,Arg^{26,34},Lys³⁹(N^{ε}-tetradecanoyl))hGLP-1(7-38)OH; | (SEQ ID NO.453) |
| (Ser⁸,Arg^{26,34},Lys³⁶(N^{ε}-tetradecanoyl),β-Ala³⁷)hGLP-1(7-37)OH; | (SEQ ID NO.454) |
| (Ser⁸,Aib³⁷,Arg^{26,34},Lys³⁶(N^{ε}-tetradecanoyl))hGLP-1(7-37)OH; | (SEQ ID NO.455) |
| (Ser⁸,Aib³⁵,Arg^{26,34},Ado³⁷)hGLP-1(7-37)OH; | (SEQ ID NO.456) |
| (Ser⁸,Aib³⁵,Arg^{26,34},Ado³⁷)hGLP-1(7-37)NH₂; | (SEQ ID NO.457) |
| (Ser⁸,Arg^{26,34},Lys³⁶(N^{ε}-tetradecanoyl),D-Ala³⁷)hGLP-1(7-37)OH; | (SEQ ID NO.458) |
| (Ser⁸,Aib³⁷,Arg^{26,34},Lys³⁸(N^{ε}-tetradecanoyl))hGLP-1(7-38)OH; | (SEQ ID NO.459) |
| (Ser⁸,Arg^{26,34},β-Ala³⁷,Lys³⁸(N^{ε}-tetradecanoyl))hGLP-1(7-38)OH; | (SEQ ID NO.460) |
| (Ser⁸,Aib³⁵,Lys²⁶(N^{ε}-octanoyl))hGLP-1(7-36)NH₂; | (SEQ ID NO.461) |
| (Ser⁸,Aib³⁵,Lys²⁶(N^{ε}-tetradecanoyl))hGLP-1(7-36)NH₂; | (SEQ ID NO.462) |
| (Ser⁸,Aib³⁵,Lys²⁶(N^{ε}-hexadecanoyl))hGLP-1(7-36)NH₂; | (SEQ ID NO.463) |
| (Ser⁸,Lys²⁶(N^{ε}-octanoyl),β-Ala³⁵)hGLP-1(7-36)NH₂; | (SEQ ID NO.464) |
| (Ser⁸,Lys²⁶(N^{ε}-tetradecanoyl),β-Ala³⁵)hGLP-1(7-36)NH₂; | (SEQ ID NO.465) |
| (Ser⁸,Lys²⁶(N^{ε}-hexadecanoyl),β-Ala³⁵)hGLP-1(7-36)NH₂; | (SEQ ID NO.466) |
| (Ser⁸,Aib³⁵,Lys²⁶(N^{ε}-octanoyl),Arg³⁴)hGLP-1(7-36)NH₂; | (SEQ ID NO.467) |
| (Ser⁸,Aib³⁵,Lys²⁶(N^{ε}-tetradecanoyl),Arg³⁴)hGLP-1(7-36)NH₂; | (SEQ ID NO.468) |
| (Ser⁸,Aib³⁵,Lys²⁶(N^{ε}-hexadecanoyl),Arg³⁴)hGLP-1(7-36)NH₂; | (SEQ ID NO.469) |
| (Ser⁸,Aib³⁵,Lys²⁶(N^{ε}-decanoyl),Arg³⁴)hGLP-1(7-36)NH₂; | (SEQ ID NO.470) |
| (Ser⁸,Aib³⁵,Lys²⁵,Lys²⁶(N^{ε}-octanoyl),Arg³⁴)hGLP-1(7-36)NH₂; | (SEQ ID NO.471) |
| (Ser⁸,Aib³⁵,Lys²⁵,Lys²⁶(N^{ε}-tetradecanoyl),Arg³⁴)hGLP-1(7-36)NH₂; | (SEQ ID NO.472) |
| (Ser⁸,Aib³⁵,Lys²⁵,Lys²⁶(N^{ε}-hexadecanoyl),Arg³⁴)hGLP-1(7-36)NH₂; | (SEQ ID NO.473) |
| (Ser⁸,Aib³⁵,Arg^{25,34},Lys²⁶(N^{ε}-octanoyl))hGLP-1(7-36)NH₂; | (SEQ ID NO.474) |
| (Ser⁸,Aib³⁵,Arg^{25,34},Lys²⁶(N^{ε}-tetradecanoyl))hGLP-1(7-36)NH₂; | (SEQ ID NO.475) |
| (Ser⁸,Aib³⁵,Arg^{25,34},Lys²⁶(N^{ε}-hexadecanoyl))hGLP-1(7-36)NH₂; | (SEQ ID NO.476) |
| (Ser⁸,Aib³⁵,Arg^{25,34},Lys²⁶(N^{ε}-decanoyl))hGLP-1(7-36)NH₂; | (SEQ ID NO.477) |
| (Ser⁸,Lys²⁶(N^{ε}-octanoyl),Arg³⁴,β-Ala³⁵)hGLP-1(7-36)NH₂; | (SEQ ID NO.478) |
| (Ser⁸,Lys²⁶(N^{ε}-tetradecanoyl),Arg³⁴,β-Ala³⁵)hGLP-1(7-36)NH₂; | (SEQ ID NO.479) |
| (Ser⁸,Lys²⁶(N^{ε}-hexadecanoyl),Arg³⁴,β-Ala³⁵)hGLP-1(7-36)NH₂; | (SEQ ID NO.480) |
| (Ser⁸,Lys²⁶(N^{ε}-decanoyl),Arg³⁴,β-Ala³⁵)hGLP-1(7-36)NH₂; | (SEQ ID NO.481) |
| (Ser⁸,Aib³⁵,Lys³⁴(N^{ε}-octanoyl))hGLP-1(7-36)NH₂; | (SEQ ID NO.482) |
| (Ser⁸,Aib³⁵,Lys³⁴(N^{ε}-tetradecanoyl))hGLP-1(7-36)NH₂; | (SEQ ID NO.483) |
| (Ser⁸,Aib³⁵,Lys³⁴(N^{ε}-hexadecanoyl))hGLP-1(7-36)NH₂; | (SEQ ID NO.484) |
| (Ser⁸,Aib³⁵,Arg²⁶,Lys³⁴(N^{ε}-octanoyl))hGLP-1(7-36)NH₂; | (SEQ ID NO.485) |
| (Ser⁸,Aib³⁵,Arg²⁶,Lys³⁴(N^{ε}-hexadecanoyl))hGLP-1(7-36)NH₂; | (SEQ ID NO.486) |
| (Ser⁸,Aib³⁵,Arg²⁶,Lys³⁴(N^{ε}-decanoyl))hGLP-1(7-36)NH₂; | (SEQ ID NO.487) |
| (Ser⁸,Aib³⁵,Arg^{25,26},Lys³⁴(N^{ε}-octanoyl))hGLP-1(7-36)NH₂; | (SEQ ID NO.488) |
| (Ser⁸,Aib³⁵,Arg^{25,26},Lys³⁴(N^{ε}-tetradecanoyl))hGLP-1(7-36)NH₂; | (SEQ ID NO.489) |
| (Ser⁸,Aib³⁵,Arg^{25,26},Lys³⁴(N^{ε}-hexadecanoyl))hGLP-1(7-36)NH₂; | (SEQ ID NO.490) |
| (Ser⁸,Aib³⁵,Arg^{25,26},Lys³⁴(N^{ε}-decanoyl))hGLP-1(7-36)NH₂; | (SEQ ID NO.491) |
| (Ser⁸,Aib³⁵,Lys²⁵,Arg²⁶,Lys³⁴(N^{ε}-octanoyl))hGLP-1(7-36)NH₂; | (SEQ ID NO.492) |
| (Ser⁸,Aib³⁵,Lys²⁵,Arg²⁶,Lys³⁴(N^{ε}-tetradecanoyl))hGLP-1(7-36)NH₂; | (SEQ ID NO.493) |
| (Ser⁸,Aib³⁵,Lys²⁵,Arg²⁶,Lys³⁴(N^{ε}-hexadecanoyl))hGLP-1(7-36)NH₂; | (SEQ ID NO.494) |
| (Ser⁸,Aib³⁵,Lys³⁶(N^{ε}-octanoyl))hGLP-1(7-36)NH₂; | (SEQ ID NO.495) |
| (Ser⁸,Aib³⁵,Lys³⁶(N^{ε}-tetradecanoyl))hGLP-1(7-36)NH₂; | (SEQ ID NO.496) |
| (Ser⁸,Aib³⁵,Lys³⁶(N^{ε}-hexadecanoyl))hGLP-1(7-36)NH₂; | (SEQ ID NO.497) |
| (Ser⁸,Aib³⁵,Arg²⁶,Lys³⁶(N^{ε}-octanoyl))hGLP-1(7-36)NH₂; | (SEQ ID NO.498) |
| (Ser⁸,Aib³⁵,Arg²⁶,Lys³⁶(N^{ε}-tetradecanoyl))hGLP-1(7-36)NH₂; | (SEQ ID NO.499) |
| (Ser⁸,Aib³⁵,Arg²⁶,Lys³⁶(N^{ε}-hexadecanoyl))hGLP-1(7-36)NH₂; | (SEQ ID NO.500) |
| (Ser⁸,Aib³⁵,Arg^{26,34},Lys³⁶(N^{ε}-octanoyl))hGLP-1(7-36)NH₂; | (SEQ ID NO.501) |
| (Ser⁸,Aib³⁵,Arg^{26,34},Lys³⁶(N^{ε}-hexadecanoyl))hGLP-1(7-36)NH₂; | (SEQ ID NO.502) |
| (Ser⁸,Aib³⁵,Arg^{26,34},Lys³⁸(N^{ε}-octanoyl))hGLP-1(7-38)NH₂; | (SEQ ID NO.503) |
| (Ser⁸,Aib³⁵,Arg^{26,34},Lys³⁸(N^{ε}-decanoyl))hGLP-1(7-38)NH₂; | (SEQ ID NO.504) |
| (Ser⁸,Aib³⁵,Arg^{26,34},Lys³⁸(N^{ε}-tetradecanoyl))hGLP-1(7-38)NH₂; | (SEQ ID NO.505) |
| (Ser⁸,Aib³⁵,Arg^{26,34},Lys³⁸(N^{ε}-hexadecanoyl))hGLP-1(7-38)NH₂; | (SEQ ID NO.506) |
| (Ser⁸,Aib^{35,37},Arg^{25,26,34},Lys³⁸(N^{ε}-octanoyl))hGLP-1(7-38)NH₂; | (SEQ ID NO.507) |
| (Ser⁸,Aib^{35,37},Arg^{25,26,34},Lys³⁸(N^{ε}-decanoyl))hGLP-1(7-38)NH₂; | (SEQ ID NO.508) |
| (Ser⁸,Aib^{35,37},Arg^{25,26,34},Lys³⁸(N^{ε}-tetradecanoyl))hGLP-1(7-38)NH₂; | (SEQ ID NO.509) |
| (Ser⁸,Aib^{35,37},Arg^{25,26,34},Lys³⁸(N^{ε}-hexadecanoyl))hGLP-1(7-38)NH₂; | (SEQ ID NO.510) |
| (Ser⁸,Aib^{35,37},Arg^{26,34},Lys³⁸(N^{ε}-octanoyl))hGLP-1(7-38)NH₂; | (SEQ ID NO.511) |
| (Ser⁸,Aib^{35,37},Arg^{26,34},Lys³⁸(N^{ε}-decanoyl))hGLP-1(7-38)NH₂; | (SEQ ID NO.512) |
| (Ser⁸,Aib^{35,37},Arg^{26,34},Lys³⁸(N^{ε}-hexadecanoyl))hGLP-1(7-38)NH₂; | (SEQ ID NO.513) |
| (Ser⁸,Aib³⁵,Lys²⁵,Arg^{26,34},Lys³⁶(N^{ε}-octanoyl))hGLP-1(7-36)NH₂; | (SEQ ID NO.514) |
| (Ser⁸,Aib³⁵,Lys²⁵,Arg^{26,34},Lys³⁶(N^{ε}-tetradecanoyl)) | (SEQ ID NO.515) |
| hGLP-1(7-36)NH₂; | |
| (Ser⁸,Aib³⁵,Lys²⁵,Arg^{26,34},Lys³⁶(N^{ε}-hexadecanoyl)) | (SEQ ID NO.516) |
| hGLP-1(7-36)NH₂; | |
| (Ser⁸,Aib³⁵,Arg^{25,26,34},Lys³⁶(N^{ε}-octanoyl))hGLP-1(7-36)NH₂; | (SEQ ID NO.517) |
| (Ser⁸,Aib³⁵,Arg^{25,26,34},Lys³⁶(N^{ε}-tetradecanoyl))hGLP-1(7-36)NH₂; | (SEQ ID NO.518) |
| (Ser⁸,Aib³⁵,Arg^{25,26,34},Lys³⁶(N^{ε}-hexadecanoyl))hGLP-1(7-36)NH₂; | (SEQ ID NO.519) |
| (Ser⁸,Aib³⁵,Arg^{25,26,34},Lys³⁵(N^{ε}-decanoyl))hGLP-1(7-36)NH₂; | (SEQ ID NO.520) |
| (Ser⁸,Lys³⁴(N^{ε}-octanoyl),β-Ala³⁵)hGLP-1(7-36)NH₂; | (SEQ ID NO.521) |
| (Ser⁸,Lys³⁴(N^{ε}-tetradecanoyl),β-Ala³⁵)hGLP-1(7-36)NH₂; | (SEQ ID NO.522) |
| (Ser⁸,Lys³⁴(N^{ε}-hexadecanoyl),β-Ala³⁵)hGLP-1(7-36)NH₂; | (SEQ ID NO.523) |
| (Ser⁸,A6c³²,Lys³⁴(N^{e}-octanoyl),β-Ala³⁵)hGLP-1(7-36)NH₂; | (SEQ ID NO.524) |
| (Ser⁸,Glu²³,Lys³⁴(N^{e}-octanoyl),β-Ala³⁵)hGLP-1(7-36)NH₂; | (SEQ ID NO.525) |
| (Ser⁸,Glu²³,A6c³²,Lys³⁴(N^{e}-octanoyl),β-Ala³⁵)hGLP-1(7-36)NH₂; | (SEQ ID NO.526) |
| (Ser⁸,Arg²⁶,Lys³⁴(N^{ε}-octanoyl),β-Ala³⁵)hGLP-1(7-36)NH₂; | (SEQ ID NO.527) |
| (Ser⁸,Arg²⁶,Lys³⁴(N^{ε}-tetradecanoyl),(β-Ala³⁵)hGLP-1(7-36)NH₂; | (SEQ ID NO.528) |
| (Ser⁸,Arg²⁶,Lys³⁴(N^{ε}-hexadecanoyl),β-Ala³⁵)hGLP-1(7-36)NH₂; | (SEQ ID NO.529) |
| (Ser⁸,Arg²⁶,Lys³⁴(N^{ε}-decanoyl),β-Ala³⁵)hGLP-1(7-36)NH₂; | (SEQ ID NO.530) |
| (Ser⁸,Arg^{25,26},Lys³⁴(N^{ε}-octanoyl),β-Ala³⁵)hGLP-1(7-36)NH₂; | (SEQ ID NO.531) |
| (Ser⁸,Arg^{25,26},Lys³⁴(N^{ε}-tetradecanoyl),β-Ala³⁵)hGLP-1(7-36)NH₂; | (SEQ ID NO.532) |
| (Ser⁸,Arg^{25,26},Lys³⁴(N^{ε}-hexadecanoyl),β-Ala³⁵)hGLP-1(7-36)NH₂; | (SEQ ID NO.533) |
| (Ser⁸,Arg^{25,26},Lys³⁴(N^{ε}-decanoyl),β-Ala³⁵)hGLP-1(7-36)NH₂; | (SEQ ID NO.534) |
| (Ser⁸,Lys²⁵,Arg²⁶,Lys³⁴(N^{ε}-octanoyl),β-Ala³⁵)hGLP-1(7-36)NH₂; | (SEQ ID NO.535) |
| (Ser⁸,Lys²⁵,Arg²⁶,Lys³⁴(N^{ε}-tetradecanoyl),β-Ala³⁵) | (SEQ ID NO.536) |
| hGLP-1(7-36)NH₂; | |
| (Ser⁸,Lys²⁵,Arg²⁶,Lys³⁴(N^{ε}-hexadecanoyl),β-Ala³⁵) | (SEQ ID NO.537) |
| hGLP-1(7-36)NH₂; | |
| (Ser⁸,β-Ala³⁵,Lys³⁶(N^{ε}-octanoyl))hGLP-1(7-36)NH₂; | (SEQ ID NO.538) |
| (Ser⁸,β-Ala³⁵,Lys³⁶(N^{ε}-tetradecanoyl))hGLP-1(7-36)NH₂; | (SEQ ID NO.539) |
| (Ser⁸,β-Ala³⁵,Lys³⁶(N^{ε}-hexadecanoyl))hGLP-1(7-36)NH₂; | (SEQ ID NO.540) |
| (Ser⁸,Arg²⁶,β-Ala³⁵,Lys³⁶(N^{ε}-octanoyl))hGLP-1(7-36)NH₂; | (SEQ ID NO.541) |
| (Ser⁸,Arg²⁶,β-Ala³⁵,Lys³⁶(N^{ε}-tetradecanoyl))hGLP-1(7-36)NH₂; | (SEQ ID NO.542) |
| (Ser⁸,Arg²⁶,β-Ala³⁵,Lys³⁶(N^{ε}-hexadecanoyl))hGLP-1(7-36)NH₂; | (SEQ ID NO.543) |
| (Ser⁸,Arg^{26,34},β-Ala³⁵,Lys³⁶(N^{ε}-octanoyl))hGLP-1(7-36)NH₂; | (SEQ ID NO.544) |
| (Ser⁸,Arg^{26,34},β-Ala³⁵,Lys³⁶(N^{ε}-tetradecanoyl))hGLP-1(7-36)NH₂; | (SEQ ID NO.545) |
| (Ser⁸,Arg^{26,34},β-Ala³⁵,Lys³⁶(N^{ε}-hexadecanoyl))hGLP-1(7-36)NH₂; | (SEQ ID NO.546) |
| (Ser⁸,Arg^{26,34},β-Ala³⁵,Lys³⁶(N^{ε}-decanoyl))hGLP-1(7-36)NH₂; | (SEQ ID NO.547) |
| (Ser⁸,Lys²⁵,Arg^{26,34},β-Ala³⁵,Lys³⁶(N^{ε}-octanoyl))hGLP-1(7-36)NH₂; | (SEQ ID NO.548) |
| (Ser⁸,Lys²⁵,Arg^{26,34},Lys³⁶(N^{ε}-tetradecanoyl),β-Ala³⁵) | (SEQ ID NO.549) |
| hGLP-1(7-36)NH₂; | |
| (Ser⁸,Lys²⁵,Arg^{26,34},β-Ala³⁵,Lys³⁶(N^{ε}-hexadecanoyl)) | (SEQ ID NO.550) |
| hGLP-1(7-36)NH₂; | |
| (Ser⁸,Arg^{25,26,34},β-Ala³⁵,Lys³⁶(N^{ε}-octanoyl))hGLP-1(7-36)NH₂; | (SEQ ID NO.551) |
| (Ser⁸,Arg^{25,26,34},β-Ala³⁵,Lys³⁶(N^{ε}-tetradecanoyl))hGLP-1(7-36)NH₂; | (SEQ ID NO.552) |
| (Ser⁸,Arg^{25,26,34},β-Ala³⁵,Lys³⁶(N^{ε}-hexadecanoyl))hGLP-1(7-36)NH₂; | (SEQ ID NO.553) |
| (Ser⁸,Arg^{25,26,34},β-Ala³⁵,Lys³⁶(N^{ε}-decanoyl))hGLP-1(7-36)NH₂; | (SEQ ID NO.554) |
| (Ser⁸,Aib³⁵,Lys²⁶(N^{ε}-octanoyl),A6c³²,Arg³⁴)hGLP-1(7-36)NH₂; | (SEQ ID NO.555) |
| (Ser⁸,Aib³⁵,Lys²⁶(N^{ε}-tetradecanoyl),A6c³²,Arg³⁴) | (SEQ ID NO.556) |
| hGLP-1(7-36)NH₂; | |
| (Ser⁸,Aib³⁵,Lys²⁶(N^{ε}-hexadecanoyl),A6c³²,Arg³⁴) | (SEQ ID NO.557) |
| hGLP-1(7-36)NH₂; | |
| (Ser⁸,Aib³⁵,A6c³²,Lys³⁴(N^{ε}-octanoyl))hGLP-1(7-36)NH₂; | (SEQ ID NO.558) |
| (Ser⁸,Aib³⁵,A6c³²,Lys³⁴(N^{ε}-tetradecanoyl))hGLP-1(7-36)NH₂; | (SEQ ID NO.559) |
| (Ser⁸,Aib³⁵,A6c³²,Lys³⁴(N^{ε}-hexadecanoyl))hGLP-1(7-36)NH₂; | (SEQ ID NO.560) |
| (Ser⁸,Aib³⁵,Arg²⁶,A6c³²,Lys³⁴(N^{ε}-octanoyl))hGLP-1(7-36)NH₂; | (SEQ ID NO.561) |
| (Ser⁸,Aib³⁵,Arg²⁶,A6c³²,Lys³⁴(N^{ε}-tetradecanoyl))hGLP-1(7-36)NH₂; | (SEQ ID NO.562) |
| (Ser⁸,Aib³⁵,A6c³²,Lys³⁶(N^{ε}-octanoyl))hGLP-1(7-36)NH₂; | (SEQ ID NO.563) |
| (Ser⁸,Aib³⁵,A6c³²,Lys³⁶(N^{ε}-tetradecanoyl))hGLP-1(7-36)NH₂; | (SEQ ID NO.564) |
| (Ser⁸,Aib³⁵,A6c³²,Lys³⁶(N^{ε}-hexadecanoyl))hGLP-1(7-36)NH₂; | (SEQ ID NO.565) |
| (Ser⁸,Aib³⁵,Arg²⁶,A6c³²,Lys³⁶(N^{ε}-octanoyl))hGLP-1(7-36)NH₂; | (SEQ ID NO.566) |
| (Ser⁸,Aib³⁵,Arg²⁶,A6c³²,Lys³⁶(N^{ε}-tetradecanoyl))hGLP-1(7-36)NH₂; | (SEQ ID NO.567) |
| (Ser⁸,Aib³⁵,Arg²⁶,A6c³²,Lys³⁶(N^{ε}-hexadecanoyl))hGLP-1(7-36)NH₂; | (SEQ ID NO.568) |
| (Ser⁸,Aib³⁵,Arg^{26,34},A6c³²,Lys³⁶(N^{ε}-octanoyl))hGLP-1(7-36)NH₂; | (SEQ ID NO.569) |
| (Ser⁸,Aib³⁵,Arg^{26,34},A6c³²,Lys³⁶(N^{ε}-decanoyl))hGLP-1(7-36)NH₂; | (SEQ ID NO.570) |
| (Ser⁸,Aib³⁵,Arg^{26,34},A6c³²,Lys³⁶(N^{ε}-tetradecanoyl)) | (SEQ ID NO.571) |
| hGLP-1(7-36)NH₂; | |
| (Ser⁸,Aib³⁵,Arg^{26,34},A6c³²,Lys³⁶(N^{ε}-hexadecanoyl)) | (SEQ ID NO.572) |
| hGLP-1(7-36)NH₂; | |
| (Ser⁸,Aib^{24,35},Lys²⁶(N^{ε}-octanoyl),Arg³⁴)hGLP-1(7-36)NH₂; | (SEQ ID NO.573) |
| (Ser⁸,Aib^{24,35},Lys²⁶(N^{ε}-tetradecanoyl),Arg³⁴)hGLP-1(7-36)NH₂; | (SEQ ID NO.574) |
| (Ser⁸,Aib^{24,35},Lys²⁶(N^{ε}-hexadecanoyl),Arg³⁴)hGLP-1(7-36)NH₂; | (SEQ ID NO.575) |
| (Ser⁸,Aib^{24,35},Arg²⁶,Lys³⁴(N^{ε}-octanoyl))hGLP-1(7-36)NH₂; | (SEQ ID NO.576) |
| (Ser⁸,Aib^{24,35},Arg²⁶,Lys³⁴(N^{ε}-tetradecanoyl))hGLP-1(7-36)NH₂; | (SEQ ID NO.577) |
| (Ser⁸,Aib^{24,35},Arg²⁶,Lys³⁴(N^{ε}-hexadecanoyl))hGLP-1(7-36)NH₂; | (SEQ ID NO.578) |
| (Ser⁸,Aib^{24,35},Arg^{26,34},Lys³⁶(N^{ε}-octanoyl))hGLP-1(7-36)NH₂; | (SEQ ID NO.579) |
| (Ser⁸,Aib^{24,35},Arg^{26,34},Lys³⁶(N^{ε}-tetradecanoyl))hGLP-1(7-36)NH₂; | (SEQ ID NO.580) |
| (Ser⁸,Aib^{24,35},Arg^{26,34},Lys³⁶(N^{ε}-hexadecanoyl))hGLP-1(7-36)NH₂; | (SEQ ID NO.581) |
| (Ser⁸,Aib^{24,35},Glu²³,A6c³²,Lys³⁴(N^{e}-octanoyl))hGLP-1(7-36)NH₂; | (SEQ ID NO.582) |
| (Ser⁸,Aib³⁵,Glu²³,Lys²⁶(N^{ε}-octanoyl),Arg³⁴)hGLP-1(7-36)NH₂; | (SEQ ID NO.583) |
| (Ser⁸,Aib³⁵,Glu²³,Lys²⁶(N^{ε}-tetradecanoyl),Arg³⁴)hGLP-1(7-36)NH₂; | (SEQ ID NO.584) |
| (Ser⁸,Aib³⁵,Glu²³,Lys²⁶(N^{ε}-hexadecanoyl),Arg³⁴)hGLP-1(7-36)NH₂; | (SEQ ID NO.585) |
| (Ser⁸,Aib³⁵,Glu²³,Lys³⁴(N^{e}-octanoyl))hGLP-1(7-36)NH₂; | (SEQ ID NO.586) |
| (Ser⁸,Aib³⁵,Glu²³,A6c³²,Lys³⁴(N^{e}-octanoyl))hGLP-1(7-36)NH₂; | (SEQ ID NO.587) |
| (Ser⁸,Aib³⁵,Glu²³,Arg²⁶,Lys³⁴(N^{ε}-octanoyl))hGLP-1(7-36)NH₂; | (SEQ ID NO.588) |
| (Ser⁸,Aib³⁵,Glu²³,Arg²⁶,Lys³⁴(N^{ε}-tetradecanoyl))hGLP-1(7-36)NH₂; | (SEQ ID NO.589) |
| (Ser⁸,Aib³⁵,Glu²³,Arg²⁶,Lys³⁴(N^{ε}-hexadecanoyl))hGLP-1(7-36)NH₂; | (SEQ ID NO.590) |
| (Ser⁸,Aib³⁵,Glu²³,Lys³⁶(N^{ε}-octanoyl))hGLP-1(7-36)NH₂; | (SEQ ID NO.591) |
| (Ser⁸,Aib³⁵,Glu²³,Lys³⁶(N^{ε}-tetradecanoyl))hGLP-1(7-36)NH₂; | (SEQ ID NO.592) |
| (Ser⁸,Aib³⁵,Glu²³,Lys³⁶(N^{ε}-hexadecanoyl))hGLP-1(7-36)NH₂; | (SEQ ID NO.593) |
| (Ser⁸,Aib³⁵,Glu²³,Arg^{26,34},Lys³⁶(N^{ε}-octanoyl))hGLP-1(7-36)NH₂; | (SEQ ID NO.594) |
| (Ser⁸,Aib³⁵,Glu²³,Arg^{26,34},Lys³⁶(N^{ε}-tetradecanoyl)) | (SEQ ID NO.595) |
| hGLP-1(7-36)NH₂; | |
| (Ser⁸,Aib³⁵,Glu²³,Arg^{26,34},Lys³⁶(N^{ε}-hexadecanoyl)) | (SEQ ID NO.596) |
| hGLP-1(7-36)NH₂; | |
| (Ser⁸,Aib^{30,35},Lys²⁶(N^{ε}-octanoyl),Arg³⁴)hGLP-1(7-36)NH₂; | (SEQ ID NO.597) |
| (Ser⁸,Aib^{30,35},Lys²⁶(N^{ε}-tetradecanoyl),Arg³⁴)hGLP-1(7-36)NH₂; | (SEQ ID NO.598) |
| (Ser⁸,Aib^{30,35},Lys²⁶(N^{ε}-hexadecanoyl),Arg³⁴)hGLP-1(7-36)NH₂; | (SEQ ID NO.599) |
| (Ser⁸,Aib^{30,35},Arg²⁶,Lys³⁴(N^{ε}-octanoyl))hGLP-1(7-36)NH₂; | (SEQ ID NO.600) |
| (Ser⁸,Aib^{30,35},Arg²⁶,Lys³⁴(N^{ε}-tetradecanoyl))hGLP-1(7-36)NH₂; | (SEQ ID NO.601) |
| (Ser⁸,Aib^{30,35},Arg²⁶,Lys³⁴(N^{ε}-hexadecanoyl))hGLP-1(7-36)NH₂; | (SEQ ID NO.602) |
| (Ser⁸,Aib^{30,35},Arg^{26,34},Lys³⁶(N^{ε}-octanoyl))hGLP-1(7-36)NH₂; | (SEQ ID NO.603) |
| (Ser⁸,Aib^{30,35},Arg^{26,34},Lys³⁶(N^{ε}-tetradecanoyl))hGLP-1(7-36)NH₂; | (SEQ ID NO.604) |
| (Ser⁸,Aib^{30,35},Arg^{26,34},Lys³⁶(N^{ε}-hexadecanoyl))hGLP-1(7-36)NH₂; | (SEQ ID NO.605) |
| (Ser⁸,Aib³⁵,Glu²³,A6c³²,Lys³⁶(N^{ε}-octanoyl))hGLP-1(7-36)NH₂; | (SEQ ID NO.606) |
| (Ser⁸,Aib³⁵,Glu²³,A6c³²,Lys³⁶(N^{ε}-tetradecanoyl))hGLP-1(7-36)NH₂; | (SEQ ID NO.607) |
| (Ser⁸,Aib³⁵,Glu²³,A6c³²,Lys³⁶(N^{ε}-hexadecanoyl))hGLP-1(7-36)NH₂; | (SEQ ID NO.608) |
| (Ser⁸,Aib³⁵,Glu²³,Arg^{26,34},A6c³²,Lys³⁶(N^{ε}-octanoyl)) | (SEQ ID NO.609) |
| hGLP-1(7-36)NH₂; | |
| (Ser⁸,Aib³⁵,Glu²³,Arg^{26,34},A6c³²,Lys³⁶(N^{ε}-tetradecanoyl)) | (SEQ ID NO.610) |
| hGLP-1(7-36)NH₂; | |
| (Ser⁸,Aib³⁵,Glu²³,Arg^{26,34},A6c³²,Lys³⁶(N^{ε}-hexadecanoyl)) | (SEQ ID NO.611) |
| hGLP-1(7-36)NH₂; | |
| (Ser⁸,Aib^{24,35},Glu²³,Arg^{26,34},A6c³²,Lys³⁶(N^{ε}-octanoyl)) | (SEQ ID NO.612) |
| hGLP-1(7-36)NH₂; | |
| (Ser⁸,Aib²⁴,34,Glu²³,Arg^{26,34},A6c³²,Lys³⁶(N^{ε}-tetradecanoyl)) | (SEQ ID NO.613) |
| hGLP-1(7-36)NH₂; | |
| (Ser⁸,Aib^{24,35},Glu²³,Arg^{26,34},A6c³²,Lys³⁶(N^{ε}-hexadecanoyl)) | (SEQ ID NO.614) |
| hGLP-1(7-36)NH₂; | |
| (Ser⁸,Aib^{24,30,35},Glu²³,Arg^{26,34},A6c³²,Lys³⁶(N^{ε}-octanoyl)) | (SEQ ID NO.615) |
| hGLP-1(7-36)NH₂; | |
| (Ser⁸,Aib^{24,30,35},Glu²³,Arg^{26,34},A6c³²,Lys³⁶(N^{ε}-tetradecanoyl)) | (SEQ ID NO.616) |
| hGLP-1(7-36)NH₂; | |
| (Ser⁸,Aib^{24,30,35},Glu²³,Arg^{26,34},A6c³²,Lys³⁶(N^{ε}-hexadecanoyl)) | (SEQ ID NO.617) |
| hGLP-1(7-36)NH₂; | |
| ((N^{α}-HEPES-His)⁷,Ser⁸,β-Ala³⁵)hGLP-1(7-36)NH₂; | (SEQ ID NO.618) |
| ((N^{α}-HEPA-His)⁷,Ser⁸,β-Ala³⁵)hGLP-1(7-36)NH₂; | (SEQ ID NO.619) |
| ((N^{α}-tetradecanoyl-His)⁷,Ser⁸,Aib³⁵)hGLP-1(7-36)NH₂; | (SEQ ID NO.620) |
| ((N^{α}-tetradecanoyl-His)⁷,Ser⁸,β-Ala³⁵)hGLP-1(7-36)NH₂; | (SEQ ID NO.621) |
| ((N^{α}-tetradecanoyl-His)⁷,Ser⁸,Aib³⁵,Arg^{26,34}) | (SEQ ID NO.622) |
| hGLP-1(7-36)NH₂; | |
| ((N^{α}-tetradecanoyl-His)⁷,Ser⁸,Arg^{26,34},β-Ala³⁵) | (SEQ ID NO.623) |
| hGLP-1(7-36)NH₂; | |
| ((N^{α}-tetradecanoyl-His)⁷,Ser⁸,Aib³⁵,Arg^{25,26,34}) | (SEQ ID NO.624) |
| hGLP-1(7-36)NH₂; | |
| ((N^{α}-tetradecanoyl-His)⁷,Ser⁸,Arg^{25,26,34},β-Ala³⁵) | (SEQ ID NO.625) |
| hGLP-1(7-36)NH₂; | |
| (Ser⁸,Aib³⁵,Lys²⁶(N^{ε}-octanesulfonyl),Arg³⁴) | (SEQ ID NO.626) |
| hGLP-1(7-36)NH₂; | |
| (Ser⁸,Aib³⁵,Lys²⁶(N^{ε}-dodecanesulfonyl),Arg³⁴) | (SEQ ID NO.627) |
| hGLP-1(7-36)NH₂; | |
| (Ser⁸,Aib³⁵,Lys²⁶(N^{ε}-hexadecanesulfonyl),Arg³⁴) | (SEQ ID NO.628) |
| hGLP-1(7-36)NH₂; | |
| (Ser⁸,Aib³⁵,Arg²⁶,Lys³⁴(N^{ε}-octanesulfonyl)) | (SEQ ID NO.629) |
| hGLP-1(7-36)NH₂; | |
| (Ser⁸,Aib³⁵,Arg²⁶,Lys³⁴(N^{ε}-dodecanesulfonyl)) | (SEQ ID NO.630) |
| hGLP-1(7-36)NH₂; | |
| (Ser⁸,Aib³⁵,Arg²⁶,Lys³⁴(N^{ε}-hexadecanesulfonyl)) | (SEQ ID NO.631) |
| hGLP-1(7-36)NH₂; | |
| (Ser⁸,Aib³⁵,Arg^{26,34},Lys³⁶(N^{ε}-octanesulfonyl)) | (SEQ ID NO.632) |
| hGLP-1(7-36)NH₂; | |
| (Ser⁸,Aib³⁵,Arg^{26,34},Lys³⁶(N^{ε}-hexadecanesulfonyl)) | (SEQ ID NO.633) |
| hGLP-1(7-36)NH₂; | |
| (Ser⁸,Aib³⁵,Asp²⁶(1-(4-decylpiperazine)),Arg³⁴) | (SEQ ID NO.634) |
| hGLP-1(7-36)NH₂; | |
| (Ser⁸,Aib³⁵,Asp²⁶(1-(4-dodecylpiperazine)),Arg³⁴) | (SEQ ID NO.635) |
| hGLP-1(7-36)NH₂; | |
| (Ser⁸,Aib³⁵,Asp²⁶(1-(4-tetradecylpiperazine)),Arg³⁴) | (SEQ ID NO.636) |
| hGLP-1(7-36)NH₂; | |
| (Ser⁸,Aib³⁵,Asp²⁶(1-(4-hexadecylpiperazine)),Arg³⁴) | (SEQ ID NO.637) |
| hGLP-1(7-36)NH₂; | |
| (Ser⁸,Aib³⁵,Arg²⁶,Asp³⁴(1-(4-decylpiperazine))) | (SEQ ID NO.638) |
| hGLP-1(7-36)NH₂; | |
| (Ser⁸,Aib³⁵,Arg²⁶,Asp³⁴(1-(4-dodecylpiperazine))) | (SEQ ID NO.639) |
| hGLP-1(7-36)NH₂; | |
| (Ser⁸,Aib³⁵,Arg²⁶,Asp³⁴(1-(4-tetradecylpiperazine))) | (SEQ ID NO.640) |
| hGLP-1(7-36)NH₂; | |
| (Ser⁸,Aib³⁵,Arg²⁶,Asp³⁴(1-(4-hexadecylpiperazine))) | (SEQ ID NO.641) |
| hGLP-1(7-36)NH₂; | |
| (Ser⁸,Aib³⁵,Arg^{26,34},Asp³⁶(1-(4-decylpiperazine))) | (SEQ ID NO.642) |
| hGLP-1(7-36)NH₂; | |
| (Ser⁸,Aib³⁵,Arg^{26,34},Asp³⁶(1-(4-dodecylpiperazine))) | (SEQ ID NO.643) |
| hGLP-1(7-36)NH₂; | |
| (Ser⁸,Aib³⁵,Arg^{26,34},Asp³⁶(1-(4-hexadecylpiperazine))) | (SEQ ID NO.644) |
| hGLP-1(7-36)NH₂; | |
| (Ser⁸,Aib³⁵,Arg^{26,34},Asp³⁸(1-(4-decylpiperazine))) | (SEQ ID NO.645) |
| hGLP-1(7-38)NH₂; | |
| (Ser⁸,Aib³⁵,Arg^{26,34},Asp³⁸(1-(4-dodecylpiperazine))) | (SEQ ID NO.646) |
| hGLP-1(7-38)NH₂; | |
| (Ser⁸,Aib³⁵,Arg^{26,34},Asp³⁸(1-(4-tetradecylpiperazine))) | (SEQ ID NO.647) |
| hGLP-1(7-38)NH₂; | |
| (Ser⁸,Aib³⁵,Arg^{26,34},Asp³⁸(1-(4-hexadecylpiperazine))) | (SEQ ID NO.648) |
| hGLP-1(7-38)NH₂; | |
| (Ser⁸,Aib^{35,37},Arg^{26,34},Asp³⁸(1-(4-decylpiperazine))) | (SEQ ID NO.649) |
| hGLP-1(7-38)NH₂; | |
| (Ser⁸,Aib^{35,37},Arg^{26,34},Asp³⁸(1-(4-dodecylpiperazine))) | (SEQ ID NO.650) |
| hGLP-1(7-38)NH₂; | |
| (Ser⁸,Aib^{35,37},Arg^{26,34},Asp³⁸(1-(4-tetradecylpiperazine))) | (SEQ ID NO.651) |
| hGLP-1(7-38)NH₂; | |
| (Ser⁸,Aib^{35,37},Arg^{26,34},Asp³⁸(1-(4-hexadecylpiperazine))) | (SEQ ID NO.652) |
| hGLP-1(7-38)NH₂; | |
| (Ser⁸,Aib³⁵,Arg^{25,34},Asp²⁶(1-(4-decylpiperazine))) | (SEQ ID NO.653) |
| hGLP-1(7-36)NH₂; | |
| (Ser⁸,Aib³⁵,Arg^{25,34},Asp²⁶(1-(4-dodecylpiperazine))) | (SEQ ID NO.654) |
| hGLP-1(7-36)NH₂; | |
| (Ser⁸,Aib³⁵,Arg^{25,34},Asp²⁶(1-(4-tetradecylpiperazine))) | (SEQ ID NO.655) |
| hGLP-1(7-36)NH₂; | |
| (Ser⁸,Aib³⁵,Arg^{25,34},Asp²⁶(1-(4-hexadecylpiperazine))) | (SEQ ID NO.656) |
| hGLP-1(7-36)NH₂; | |
| (Ser⁸,Aib³⁵,Arg^{25,26},Asp³⁴(1-(4-decylpiperazine))) | (SEQ ID NO.657) |
| hGLP-1(7-36)NH₂; | |
| (Ser⁸,Aib³⁵,Arg^{25,26},Asp³⁴(1-(4-dodecylpiperazine))) | (SEQ ID NO.658) |
| hGLP-1(7-36)NH₂; | |
| (Ser⁸,Aib³⁵,Arg^{25,26},Asp³⁴(1-(4-tetradecylpiperazine))) | (SEQ ID NO.659) |
| hGLP-1(7-36)NH₂; | |
| (Ser⁸,Aib³⁵,Arg^{25,26},Asp³⁴(1-(4-hexadecylpiperazine))) | (SEQ ID NO.660) |
| hGLP-1(7-36)NH₂; | |
| (Ser⁸,Aib³⁵,Arg^{25,26,34},Asp³⁶(1-(4-decylpiperazine))) | (SEQ ID NO.661) |
| hGLP-1(7-36)NH₂; | |
| (Ser⁸,Aib³⁵,Arg^{25,26,34},Asp³⁶(1-(4-dodecylpiperazine))) | (SEQ ID NO.662) |
| hGLP-1(7-36)NH₂; | |
| (Ser⁸,Aib³⁵,Arg^{25,26,34},Asp³⁶(1-(4-tetradecylpiperazine))) | (SEQ ID NO.663) |
| hGLP-1(7-36)NH₂; | |
| (Ser⁸,Aib³⁵,Arg^{25,26,34},Asp³⁶(1-(4-hexadecylpiperazine))) | (SEQ ID NO.664) |
| hGLP-1(7-36)NH₂; | |
| (Ser⁸,Aib³⁵,Arg^{25,26,34},Asp³⁸(1-(4-decylpiperazine))) | (SEQ ID NO.665) |
| hGLP-1(7-38)NH₂; | |
| (Ser⁸,Aib³⁵,Arg^{25,26,34},Asp³⁸(1-(4-dodecylpiperazine))) | (SEQ ID NO.666) |
| hGLP-1(7-38)NH₂; | |
| (Ser⁸,Aib³⁵,Arg^{25,26,34},Asp³⁸(1-(4-tetradecylpiperazine))) | (SEQ ID NO.667) |
| hGLP-1(7-38)NH₂; | |
| (Ser⁸,Aib³⁵,Arg^{25,26,34},Asp³⁸(1-(4-hexadecylpiperazine))) | (SEQ ID NO.668) |
| hGLP-1(7-38)NH₂; | |
| (Ser⁸,Aib^{35,37},Arg^{25,26,34},Asp³⁸(1-(4-decylpiperazine))) | (SEQ ID NO.669) |
| hGLP-1(7-38)NH₂; | |
| (Ser⁸,Aib^{35,37},Arg^{25,26,34},Asp³⁸(1-(4-dodecylpiperazine))) | (SEQ ID NO.670) |
| hGLP-1(7-38)NH₂; | |
| (Ser⁸,Aib^{35,37},Arg^{25,26,34},Asp³⁸(1-(4-tetradecylpiperazine))) | (SEQ ID NO.671) |
| hGLP-1(7-38)NH₂; | |
| (Ser⁸,Aib^{35,37},Arg^{25,26,34},Asp³⁸(1-(4-hexadecylpiperazine))) | (SEQ ID NO.672) |
| hGLP-1(7-38)NH₂; | |
| Ser⁸,Aib³⁵,Arg^{26,34},Glu³⁶(1-dodecylamino)) | (SEQ ID NO.673) |
| hGLP-1(7-36)NH₂; | |
| (Ser⁸,Aib³⁵,Glu²⁶(1-dodecylamino),Arg³⁴) | (SEQ ID NO.674) |
| hGLP-1(7-36)NH₂; | |
| (Ser⁸,Aib³⁵,Arg²⁶,Glu³⁴(1-dodecylamino)) | (SEQ ID NO.675) |
| hGLP-1(7-36)NH₂; | |
| (Ser⁸,Aib^{35,37},Arg^{26,34},Glu³⁸(1-dodecylamino)) | (SEQ ID NO.676) |
| hGLP-1(7-38)NH₂; | |
| (Ser⁸,Aib³⁵,Arg³⁴,Lys²⁶(N^{ε}-(2-(4-decyl-1-piperazine)-acetyl))) | (SEQ ID NO.677) |
| hGLP-1(7-36)NH₂; | |
| (Ser⁸,Aib³⁵,Arg³⁴,Lys²⁶(N^{ε}-(2-(4-dodecyl-1-piperazine)-acetyl))) | (SEQ ID NO.678) |
| hGLP-1(7-36)NH₂; | |
| (Ser⁸,Aib³⁵,Arg³⁴,Lys²⁶(N^{ε}-(2-(4-tetradecyl-1-piperazine)-acetyl))) | (SEQ ID NO.679) |
| hGLP-1(7-36)NH₂; | |
| (Ser⁸,Aib³⁵,Arg³⁴,Lys²⁶(N^{ε}-(2-(4-hexadecyl-1-piperazine)-acetyl))) | (SEQ ID NO.680) |
| hGLP-1(7-36)NH₂; | |
| (Ser⁸,Aib³⁵,Arg²⁶,Lys³⁴(N^{ε}-(2-(4-decyl-1-piperazine)-acetyl))) | (SEQ ID NO.681) |
| hGLP-1(7-36)NH₂; | |
| (Ser⁸,Aib³⁵,Arg²⁶,Lys³⁴(N^{ε}-(2-(4-dodecyl-1-piperazine)-acetyl)) | (SEQ ID NO.682) |
| hGLP-1(7-36)NH₂; | |
| (Ser⁸,Aib³⁵,Arg²⁶,Lys³⁴(N^{ε}-(2-(4-tetradecyl-1-piperazine)-acetyl))) | (SEQ ID NO.683) |
| hGLP-1(7-36)NH₂; | |
| (Ser⁸,Aib³⁵,Arg²⁶,Lys³⁴(N^{ε}-(2-(4-hexadecyl-1-piperazine)-acetyl))) | (SEQ ID NO.684) |
| hGLP-1(7-36)NH₂; | |
| (Ser⁸,Aib³⁵,Arg^{26,34},Lys³⁶(N^{ε}-(2-(4-decyl-1-piperazine)-acetyl))) | (SEQ ID NO.685) |
| hGLP-1(7-36)NH₂; | |
| (Ser⁸,Aib³⁵,Arg^{26,34},Lys³⁶(N^{ε}-(2-(4-dodecyl-1-piperazine)-acetyl))) | (SEQ ID NO.686) |
| hGLP-1(7-36)NH₂; | |
| (Ser⁸,Aib³⁵,Arg^{26,34},Lys³⁶(N^{ε}-(2-(4-hexadecyl-1-piperazine)-acetyl))) | (SEQ ID NO.687) |
| hGLP-1(7-36)NH₂; | |
| (Ser⁸,Aib³⁵,Arg^{26,34},Lys³⁸(N^{ε}-(2-(4-decyl-1-piperazine)-acetyl))) | (SEQ ID NO.688) |
| hGLP-1(7-38)NH₂; | |
| (Ser⁸,Aib³⁵,Arg^{26,34},Lys³⁸(N^{ε}-(2-(4-dodecyl-1-piperazine)-acetyl))) | (SEQ ID NO.689) |
| hGLP-1(7-38)NH₂; | |
| (Ser⁸,Aib³⁵,Arg^{26,34},Lys³⁸(N^{ε}-(2-(4-tetradecyl-1-piperazine)-acetyl))) | (SEQ ID NO.690) |
| hGLP-1(7-38)NH₂; | |
| (Ser⁸,Aib³⁵,Arg^{26,34},Lys³⁸(N^{ε}-(2-(4-hexadecyl-1-piperazine)-acetyl))) | (SEQ ID NO.691) |
| hGLP-1(7-38)NH₂; | |
| (Ser⁸,Aib^{35,37},Arg^{26,34},Lys³⁸(N^{ε}-(2-(4-decyl-1-piperazine)-acetyl))) | (SEQ ID NO.692) |
| hGLP-1(7-38)NH₂; | |
| (Ser⁸,Aib^{35,37},Arg^{26,34},Lys³⁸(N^{ε}-(2-(4-dodecyl-1-piperazine)-acetyl))) | (SEQ ID NO.693) |
| hGLP-1(7-38)NH₂; | |
| (Ser⁸,Aib^{35,37},Arg^{26,34},Lys³⁸(N^{ε}-(2-(4-tetradecyl-1-piperazine)-acetyl))) | (SEQ ID NO.694) |
| hGLP-1(7-38)NH₂; | |
| (Ser⁸,Aib^{35,37},Arg^{26,34},Lys³⁸(N^{ε}-(2-(4-hexadecyl-1-piperazine)-acetyl))) | (SEQ ID NO.695) |
| hGLP-1(7-38)NH₂; | |
| (Ser⁸,Aib³⁵,Arg^{25,34},Lys²⁶(N^{ε}-(2-(4-decyl-1-piperazine)-acetyl))) | (SEQ ID NO.696) |
| hGLP-1(7-36)NH₂; | |
| (Ser⁸,Aib³⁵,Arg^{25,34},Lys²⁶(N^{ε}-(2-(4-dodecyl-1-piperazine)-acetyl))) | (SEQ ID NO.697) |
| hGLP-1(7-36)NH₂; | |
| (Ser⁸,Aib³⁵,Arg^{25,34},Lys²⁶(N^{ε}-(2-(4-tetradecyl-1-piperazine)-acetyl))) | (SEQ ID NO.698) |
| hGLP-1(7-36)NH₂; | |
| (Ser⁸,Aib³⁵,Arg^{25,34},Lys²⁶(N^{ε}-(2-(4-hexadecyl-1-piperazine)-acetyl))) | (SEQ ID NO.699) |
| hGLP-1(7-36)NH₂; | |
| (Ser⁸,Aib³⁵,Arg^{25,26},Lys³⁴(N^{ε}-(2-(4-decyl-1-piperazine)-acetyl))) | (SEQ ID NO.700) |
| hGLP-1(7-36)NH₂; | |
| (Ser⁸,Aib³⁵,Arg^{25,26},Lys³⁴(N^{ε}-(2-(4-dodecyl-1-piperazine)-acetyl))) | (SEQ ID NO.701) |
| hGLP-1(7-36)NH₂; | |
| (Ser⁸,Aib³⁵,Arg^{25,26},Lys³⁴(N^{ε}-(2-(4-tetradecyl-1-piperazine)-acetyl))) | (SEQ ID NO.702) |
| hGLP-1(7-36)NH₂; | |
| (Ser⁸,Aib³⁵,Arg^{25,26},Lys³⁴(N^{ε}-(2-(4-hexadecyl-1-piperazine)-acetyl))) | (SEQ ID NO.703) |
| hGLP-1(7-36)NH₂; | |
| (Ser⁸,Aib³⁵,Arg^{25,26,34},Lys³⁶(N^{ε}-(2-(4-decyl-1-piperazine)-acetyl))) | (SEQ ID NO.704) |
| hGLP-1(7-36)NH₂; | |
| (Ser⁸,Aib³⁵,Arg2526,34,Lys³⁶(N^{ε}-(2-(4-dodecyl-1-piperazine)-acetyl))) | (SEQ ID NO.705) |
| hGLP-1(7-36)NH₂; | |
| (Ser⁸,Aib³⁵,Arg^{25,26,34},Lys³⁶(N^{ε}-(2-(4-tetradecyl-1-piperazine)-acetyl))) | (SEQ ID NO.706) |
| hGLP-1(7-36)NH₂; | |
| (Ser⁸,Aib³⁵,Arg^{25,26,34},Lys³⁶(N^{ε}-(2-(4-hexadecyl-1-piperazine)-acetyl))) | (SEQ ID NO.707) |
| hGLP-1(7-36)NH₂; | |
| (Ser⁸,Aib³⁵,Arg^{25,26,34},Lys³⁸(N^{ε}-(2-(4-decyl-1-piperazine)-acetyl))) | (SEQ ID NO.708) |
| hGLP-1(7-38)NH₂; | |
| (Ser⁸,Aib³⁵,Arg^{25,26,34},Lys³⁸(N^{ε}-(2-(4-dodecyl-1-piperazine)-acetyl))) | (SEQ ID NO.709) |
| hGLP-1(7-38)NH₂; | |
| (Ser⁸,Aib³⁵,Arg^{25,26,34},Lys³⁸(N^{ε}-(2-(4-tetradecyl-1-piperazine)-acetyl))) | (SEQ ID NO.710) |
| hGLP-1(7-38)NH₂; | |
| (Ser⁸,Aib³⁵,Arg^{25,26,34},Lys³⁸(N^{ε}-(2-(4-hexadecyl-1-piperazine)-acetyl))) | (SEQ ID NO.711) |
| hGLP-1(7-38)NH₂; | |
| (Ser⁸,Aib^{35,37},Arg^{25,26,34},Lys³⁸(N^{ε}-(2-(4-decyl-1-piperazine)-acetyl))) | (SEQ ID NO.712) |
| hGLP-1(7-38)NH₂; | |
| (Ser⁸,Aib^{35,37},Arg^{25,26,34},Lys³⁸(N^{ε}-(2-(4-dodecyl-1-piperazine)-acetyl))) | (SEQ ID NO.713) |
| hGLP-1(7-38)NH₂; | |
| (Ser⁸,Aib^{35,37},Arg^{25,26,34},Lys³⁸(N^{ε}-(2-(4-tetradecyl-1-piperazine)-acetyl))) | (SEQ ID NO.714) |
| hGLP-1(7-38)NH₂; | |
| (Ser⁸,Aib^{35,37},Arg^{25,26,34},Lys³⁸(N^{ε}-(2-(4-hexadecyl-1-piperazine)-acetyl))) | (SEQ ID NO.715) |
| hGLP-1(7-38)NH₂; | |
| (Ser⁸,Aib³⁵,Arg^{26,34},Lys³⁶(N^{ε}-decanoyl))hGLP-1(7-36)OH; | (SEQ ID NO.716) |
| (Ser⁸,Aib³⁵,Lys²⁵,Arg^{26,34},Lys³⁶(N^{ε}-decanoyl))hGLP-1(7-36)OH; | (SEQ ID NO.717) |
| (Ser⁸,Aib³⁵,Arg^{26,34},Ava³⁷,Ado³⁸)hGLP-1(7-38)NH₂; | (SEQ ID NO.718) |
| (Ser⁸,Aib³⁵,Arg^{26,34},Asp³⁷,Ava³⁸,Ado³⁹)hGLP-1(7-39)NH₂; | (SEQ ID NO.719) |
| (Ser⁸,Aib³⁵,Arg^{26,34},Aun³⁷)hGLP-1(7-37)NH₂; | (SEQ ID NO.720) |
| (Ser⁸,Aib^{17,35})hGLP-1(7-36)NH₂; | (SEQ ID NO.721) |
| (Ser⁸,Arg^{26,34},β-Ala³⁵,D-Asp³⁷,Ava³⁸,Aun³⁹)hGLP-1(7-39)NH₂; | (SEQ ID NO.722) |
| (Ser⁸,Glu^{22,23},β-Ala³⁵)hGLP-1(7-36)NH₂; | (SEQ ID NO.723) |
| (Ser⁸,Lys¹⁸,β-Ala³⁵)hGLP-1(7-36)NH₂; | (SEQ ID NO.724) |
| (Ser⁸,Leu²⁷,β-Ala³⁵)hGLP-1(7-36)NH₂; | (SEQ ID NO.725) |
| (Ser⁸,Lys³³,β-Ala³⁵)hGLP-1(7-36)NH₂; | (SEQ ID NO.726) |
| (Ser⁸,Lys¹⁸,Leu²⁷,β-Ala³⁵)hGLP-1(7-36)NH₂; | (SEQ ID NO.727) |
| (Ser⁸,D-Arg³⁶)hGLP-1(7-36)NH₂; | (SEQ ID NO.728) |
| (Ser⁸,β-Ala³⁵,D-Arg³⁷)hGLP-1(7-37)NH₂; | (SEQ ID NO.729) |
| (Ser⁸,Aib²⁷,β-Ala³⁵)hGLP-1(7-36)NH₂; | (SEQ ID NO.730) |
| (Ser⁸,Aib²⁷,β-Ala^{35,37},Arg³⁸)hGLP-1(7-38)NH₂; | (SEQ ID NO.731) |
| (Ser⁸,Aib²⁷,β-Ala^{35,37},Arg^{38,39})hGLP-1(7-39)NH₂; | (SEQ ID NO.732) |
| (Ser⁸,Lys¹⁸,27,β-Ala³⁵)hGLP-1(7-36)NH₂; | (SEQ ID NO.733) |
| (Ser⁸,Lys²⁷,β-Ala³⁵)hGLP-1(7-36)NH₂; | (SEQ ID NO.734) |
| (Ser⁸,β-Ala³⁵,Arg³⁸)hGLP-1(7-38)NH₂; | (SEQ ID NO.735) |
| (Ser⁸,Arg^{26,34},β-Ala³⁵)hGLP-1(7-36)NH₂; | (SEQ ID NO.736) |
| (Ser⁸,D-Arg³⁵)hGLP-1(7-36)NH₂; | (SEQ ID NO.737) |
| (Ser⁸,β-Ala³⁵,Arg³⁷)hGLP-1(7-37)NH₂; | (SEQ ID NO.738) |
| (Ser⁸,Phe³¹,β-Ala³⁵)hGLP-1(7-36)NH₂; | (SEQ ID NO.739) |
| (Ser⁸,Aib³⁵,Phe³¹)hGLP-1(7-36)NH₂; | (SEQ ID NO.740) |
| (Ser⁸,Aib³⁵,2-Nal³¹)hGLP-1(7-36)NH₂; | (SEQ ID NO.741) |
| (Ser⁸,Aib³⁵,2-Nal^{28,31})hGLP-1(7-36)NH₂; | (SEQ ID NO.742) |
| (Ser⁸,Aib³⁵,Arg^{26,34},2-Nal³¹)hGLP-1(7-36)NH₂; | (SEQ ID NO.743) |
| (Ser⁸,Aib³⁵,Arg^{26,34},Phe³¹)hGLP-1(7-36)NH₂; | (SEQ ID NO.744) |
| (Ser⁸,Aib³⁵,2-Nal^{19,31})hGLP-1(7-36)NH₂; | (SEQ ID NO.745) |
| (Ser⁸,Aib³⁵,2-Nal^{12,31})hGLP-1(7-36)NH₂; | (SEQ ID NO.746) |
| (Ser⁸,Aib³⁵,Lys³⁶(N^{ε}-decanoyl))hGLP-1(7-36)NH₂; | (SEQ ID NO.747) |
| (Ser⁸,Aib³⁵,Arg³⁴,Lys²⁶(N^{ε}-decanoyl))hGLP-1(7-36)NH₂; | (SEQ ID NO.748) |
| (Ser⁸,Aib³⁵,Arg^{26,34},Lys³⁶(N^{ε}-dodecanoyl))hGLP-1(7-36)NH₂; | (SEQ ID NO.749) |
| (Ser⁸,β-Ala³⁵,Ser³⁷(O-decanoyl))hGLP1(7-37)-NH₂; | (SEQ ID NO.750) |
| (Ser⁸,Aib²⁷,β-Ala^{35,37},Arg³⁸,Lys³⁹(N^{ε}-octanoyl))hGLP-1(7-39)NH₂; | (SEQ ID NO.751) |
| (Ser⁸,Arg^{26,34},β-Ala³⁵,Lys³⁷(N^{ε}-octanoyl))hGLP-1(7-37)NH₂; | (SEQ ID NO.752) |
| (Ser⁸,Arg^{26,34},β-Ala³⁵,Lys³⁷(N^{ε}-decanoyl))hGLP-1(7-37)NH₂; | (SEQ ID NO.753) |
| (Ser⁸,Arg^{26,34},β-Ala³⁵,Lys³⁷(N^{ε}-tetradecanoyl))hGLP-1(7-37)NH₂; | (SEQ ID NO.754) |
| (Ser⁸,Arg^{26,34},β-Ala³⁵,Lys³⁷(N^{ε}-dodecanoyl))hGLP-1(7-37)NH₂; | (SEQ ID NO.755) |
| (Ser⁸,Arg^{26,34},β-Ala³⁵,Lys³⁷(N^{ε}-dodecanoyl))hGLP-1(8-37)NH₂; | (SEQ ID NO.756) |
| (Ser⁸,Arg^{26,34},β-Ala³⁵,Lys³⁶(N^{ε}-Aec-decanoyl))hGLP-1(7-36)NH₂; | (SEQ ID NO.757) |
| (Ser⁸,Arg²⁶,Aib³⁵)hGLP-1(7-36)NH₂; | (SEQ ID NO.758) |
| (Ser⁸,Arg²⁶,Phe³¹,Aib³⁵)hGLP-1(7-36)NH₂; | (SEQ ID NO.759) |
| (Ser⁸,Arg²⁶,Phe³¹,β-Ala³⁵)hGLP-1(7-36)NH₂; | (SEQ ID NO.760) |
| (Ser⁸,Arg²⁶,β-Ala³⁵)hGLP-1(7-36)NH₂; | (SEQ ID NO.761) |
| (Ser⁸,Arg^{26,34},Phe³¹,β-Ala³⁵)hGLP-1(7-36)NH₂; | (SEQ ID NO.762) |
| (Ser⁸,Arg³⁴,Aib³⁵)hGLP-1(7-36)NH₂; | (SEQ ID NO.763) |
| (Ser⁸,Arg³⁴,β-Ala³⁵)hGLP-1(7-36)NH₂; | (SEQ ID NO.764) |
| (Ser⁸,Phe³¹,Arg³⁴,Aib³⁵)hGLP-1(7-36)NH₂; | (SEQ ID NO.765) |
| or | |
| (Ser⁸,Phe³¹,Arg³⁴,β-Ala³⁵)hGLP-1(7-36)NH₂; | (SEQ ID NO.766) |

or a pharmaceutically acceptable salt thereof.

13. A compound according to claim 11 wherein said compound is:

| | |
|---|---|
| (Gly⁸,Aib³⁵,A6c³²)hGLP-1(7-36)NH₂; | (SEQ ID NO.8) |
| (Gly⁸,Aib³⁵,Glu²³)hGLP-1(7-36)NH₂; | (SEQ ID NO.10) |
| (Gly⁸,Aib^{24,35})hGLP-1(7-36)NH₂; | (SEQ ID NO.11) |
| (Gly⁸,Aib³⁵,Glu²³,A6c³²)hGLP-1(7-36)NH₂; | (SEQ ID NO.28) |
| (Gly⁸,Glu²³,β-Ala³⁵)hGLP-1(7-36)NH₂; | (SEQ ID NO.34) |
| (Gly⁸,Aib³⁵,Arg^{26,34})hGLP-1(7-36)NH₂; | (SEQ ID NO.59) |
| (Gly⁸,Aib³⁵,Arg^{26,34},Lys³⁶(N^{ε}-octanoyl))hGLP-1(7-36)NH₂; | (SEQ ID NO.117) |
| (Gly⁸,Aib³⁵,Arg^{26,34},Lys³⁶(N^{ε}-decanoyl))hGLP-1(7-36)OH; | (SEQ ID NO.332) |
| (Gly⁸,Aib³⁵,Lys²⁵,Arg^{26,34},Lys³⁶(N^{ε}-decanoyl))hGLP-1(7-36)OH; | (SEQ ID NO.333) |
| (Gly⁸,Arg^{26,34},β-Ala³⁵,Lys³⁶(N^{ε}-Aec-decanoyl))hGLP-1(7-36)NH₂; | (SEQ ID NO.383) |
| (Gly⁸,Aib³⁵,Arg^{26,34},Ava³⁷,Ado³⁸)hGLP-1(7-38)NH₂; | (SEQ ID NO.334) |
| (Gly⁸,Aib³⁵,Arg^{26,34},Asp³⁷,Ava³⁵,Ado³⁹)hGLP-1(7-39)NH₂; | (SEQ ID NO.335) |
| (Gly⁸,Aib³⁵,Arg^{26,34},Aun³⁷)hGLP-1(7-37)NH₂; | (SEQ ID NO.336) |
| (Gly⁸,Aib^{17,35})hGLP-1(7-36)NH₂; | (SEQ ID NO.337) |
| (Gly⁸,Arg^{26,34},β-Ala³⁵,D-Asp³⁷,Ava³⁸,Aun³⁹)hGLP-1(7-39)NH₂; | (SEQ ID NO.338) |
| (Gly⁸,Glu^{22,23},β-Ala³⁵)hGLP-1(7-36)NH₂; | (SEQ ID NO.339) |
| (Gly⁸,Lys¹⁸,β-Ala³⁵)hGLP-1(7-36)NH₂; | (SEQ ID NO.340) |
| (Gly⁸,Leu²⁷,β-Ala³⁵)hGLP-1(7-36)NH₂; | (SEQ ID NO.341) |
| (Gly⁸,Lys³³,β-Ala³⁵)hGLP-1(7-36)NH₂; | (SEQ ID NO.342) |
| (Gly⁸,Lys¹⁸,Leu²⁷,β-Ala³⁵)hGLP-1(7-36)NH₂; | (SEQ ID NO.343) |
| (Gly⁸,D-Arg³⁶)hGLP-1(7-36)NH₂; | (SEQ ID NO.344) |
| (Gly⁸,β-Ala³⁵,D-Arg³⁷)hGLP-1(7-37)NH₂; | (SEQ ID NO.345) |
| (Gly⁸,Aib²⁷,β-Ala³⁵)hGLP-1(7-36)NH₂; | (SEQ ID NO.346) |
| (Gly⁸,Aib²⁷,β-Ala^{35,37},Arg³⁸)hGLP-1(7-38)NH₂; | (SEQ ID NO.347) |
| (Gly⁸,Aib²⁷,β-Ala^{35,37},Arg^{38,39})hGLP-1(7-39)NH₂; | (SEQ ID NO.348) |
| (Gly⁸,Lys^{18,27},β-Ala³⁵)hGLP-1(7-36)NH₂; | (SEQ ID NO.349) |
| (Gly⁸,Lys²⁷,β-Ala³⁵)hGLP-1(7-36)NH₂; | (SEQ ID NO.350) |
| (Gly⁸,β-Ala³⁵,Arg³⁸)hGLP-1(7-38)NH₂; | (SEQ ID NO.351) |
| (Gly⁸,Arg^{26,34},β-Ala³⁵,)hGLP-1(7-36)NH₂; | (SEQ ID NO.352) |
| (Gly⁸,D-Arg³⁵)hGLP-1(7-36)NH₂; | (SEQ ID NO.353) |
| (Gly⁸,β-Ala³⁵,Arg³⁷)hGLP-1(7-37)NH₂; | (SEQ ID NO.354) |
| (Gly⁸,Phe³¹,β-Ala³⁵)hGLP-1(7-36)NH₂; | (SEQ ID NO.355) |
| (Gly⁸,Aib³⁵,Phe³¹)hGLP-1(7-36)NH₂; | (SEQ ID NO.356) |
| (Gly⁸,Aib³⁵,2-Nal³¹)hGLP-1(7-36)NH₂; | (SEQ ID NO.357) |
| (Gly⁸,Aib³⁵,2-Nal^{28,31})hGLP-1(7-36)NH₂; | (SEQ ID NO.358) |
| (Gly⁸,Aib³⁵,Arg^{26,34},2-Nal³¹)hGLP-1(7-36)NH₂; | (SEQ ID NO.359) |
| (Gly⁸,Aib³⁵,Arg^{26,34},Phe³¹)hGLP-1(7-36)NH₂; | (SEQ ID NO.360) |
| (Gly⁸,Aib³⁵,2-Nal^{19,31})hGLP-1(7-36)NH₂; | (SEQ ID NO.361) |
| (Gly⁸,Aib³⁵,2-Nal^{12,31})hGLP-1(7-36)NH₂; | (SEQ ID NO.362) |
| (Gly⁸,Aib³⁵,Lys³⁶(N^{ε}-decanoyl))hGLP-1(7-36) NH₂; | (SEQ ID NO.99) |
| (Gly⁸,Aib³⁵,Arg³⁴,Lys²⁶(N^{ε}-decanoyl))hGLP-1(7-36)NH₂; | (SEQ ID NO.364) |
| (Gly⁸,Aib³⁵,Arg^{26,34},Lys³⁶(N^{ε}-dodecanoyl))hGLP-1(7-36)NH₂; | (SEQ ID NO.365) |
| (Gly⁸,β-Ala³⁵,Ser³⁷(O-decanoyl))hGLP1(7-37)-NH₂; | (SEQ ID NO.384) |
| (Gly⁸,Aib²⁷,β-Ala^{35,37},Arg³⁸,Lys³⁹(N^{ε}-octanoyl))hGLP-1(7-39)NH₂; | (SEQ ID NO.367) |
| (Gly⁸,Arg^{26,34},β-Ala³⁵,Lys³⁷(N^{ε}-octanoyl))hGLP-1(7-37)NH₂; | (SEQ ID NO.368) |
| (Gly⁸,Arg^{26,34},β-Ala³⁵,Lys³⁷(N^{ε}-decanoyl))hGLP-1(7-37)NH₂; | (SEQ ID NO.369) |
| or | |
| (Gly⁸,Arg^{26,34},β-Ala³⁵,Lys³⁷(N^{ε}-tetradecanoyl))hGLP-1(7-37)NH₂; | (SEQ ID NO.370) |

or a pharmaceutically acceptable salt thereof.

14. A compound according to claim 12 wherein said compound is:

| | |
|---|---|
| (Ser⁸,Aib³⁵,A6c³²)hGLP-1(7-36)NH₂; | (SEQ ID NO.392) |
| (Ser⁸,Aib³⁵,Glu²³)hGLP-1(7-36)NH₂; | (SEQ ID NO.394) |
| (Ser⁸,Aib^{24,35})hGLP-1(7-36)NH₂; | (SEQ ID NO.395) |
| (Ser⁸,Aib³⁵,Glu²³,A6c³²)hGLP-1(7-36)NH₂; | (SEQ ID NO.412) |
| (Ser⁸,Glu²³,β-Ala³⁵)hGLP-1(7-36)NH₂; | (SEQ ID NO.418) |
| (Ser⁸,Aib³⁵,Arg^{26,34})hGLP-1(7-36)NH₂; | (SEQ ID NO.443) |
| (Ser⁸,Aib³⁵,Arg^{26,34},Lys³⁶(N^{ε}-octanoyl))hGLP-1(7-36)NH₂; | (SEQ ID NO.501) |
| (Ser⁸,Aib³⁵,Arg^{26,34},Lys³⁶(N^{ε}-decanoyl))hGLP-1(7-36)OH; | (SEQ ID NO.716) |
| (Ser⁸,Aib³⁵,Lys²⁵,Arg^{26,34},Lys³⁶(N^{ε}-decanoyl))hGLP-1(7-36)OH; | (SEQ ID NO.717) |
| (Ser⁸,Arg^{26,34},β-Ala³⁵,Lys³⁶(N^{ε}-Aec-decanoyl))hGLP-1(7-36)NH₂; | (SEQ ID NO.757) |
| (Ser⁸,Aib³⁵,Arg^{26,34},Ava³⁷,Ado³⁸)hGLP-1(7-38)NH₂; | (SEQ ID NO.718) |
| (Ser⁸,Aib³⁵,Arg^{26,34},Asp³⁷,Ava³⁵,Ado³⁹)hGLP-1(7-39)NH₂; | (SEQ ID NO.719) |
| (Ser⁸,Aib³⁵,Arg^{26,34},Aun³⁷)hGLP-1(7-37)NH₂; | (SEQ ID NO.720) |
| (Ser⁸,Aib^{17,35})hGLP-1(7-36)NH₂; | (SEQ ID NO.389) |
| (Ser⁸,Arg^{26,34},β-Ala³⁵,D-Asp³⁷,Ava³⁸,Aun³⁹)hGLP-1(7-39)NH₂; | (SEQ ID NO.722) |
| (Ser⁸,Glu^{22,23},β-Ala³⁵)hGLP-1(7-36)NH₂; | (SEQ ID NO.723) |
| (Ser⁸,Lys¹⁸,β-Ala³⁵)hGLP-1(7-36)NH₂; | (SEQ ID NO.724) |
| (Ser⁸,Leu²⁷,β-Ala³⁵)hGLP-1(7-36)NH₂; | (SEQ ID NO.725) |
| (Ser⁸,Lys³³,β-Ala³⁵)hGLP-1(7-36)NH₂; | (SEQ ID NO.726) |
| (Ser⁸,Lys¹⁸,Leu²⁷,β-Ala³⁵)hGLP-1(7-36)NH₂; | (SEQ ID NO.727) |
| (Ser⁸,D-Arg³⁶)hGLP-1(7-36)NH₂; | (SEQ ID NO.728) |
| (Ser⁸,β-Ala³⁵,D-Arg³⁷)hGLP-1(7-37)NH₂; | (SEQ ID NO.729) |
| (Ser⁸,Aib²⁷,β-Ala³⁵)hGLP-1(7-36)NH₂; | (SEQ ID NO.730) |
| (Ser⁸,Aib²⁷,β-Ala^{35,37},Arg³⁸)hGLP-1(7-38)NH₂; | (SEQ ID NO.731) |
| (Ser⁸,Aib²⁷,β-Ala^{35,37},Arg^{38,39})hGLP-1(7-39)NH₂; | (SEQ ID NO.732) |
| (Ser⁸,Lys^{18,27},β-Ala³⁵)hGLP-1(7-36)NH₂; | (SEQ ID NO.733) |
| (Ser⁸,Lys²⁷,β-Ala³⁵)hGLP-1(7-36)NH₂; | (SEQ ID NO.734) |
| (Ser⁸,β-Ala³⁵,Arg³⁸)hGLP-1(7-38)NH₂; | (SEQ ID NO.735) |
| (Ser⁸,Arg^{26,34},β-Ala³⁵,)hGLP-1(7-36)NH₂; | (SEQ ID NO.736) |
| (Ser⁸,D-Arg³⁵)hGLP-1(7-36)NH₂; | (SEQ ID NO.737) |
| (Ser⁸,β-Ala³⁵,Arg³⁷)hGLP-1(7-37)NH₂; | (SEQ ID NO.738) |
| (Ser⁸,Phe³¹,β-Ala³⁵)hGLP-1(7-36)NH₂; | (SEQ ID NO.739) |
| (Ser⁸,Aib³⁵,Phe³¹)hGLP-1(7-36)NH₂; | (SEQ ID NO.740) |
| (Ser⁸,Aib³⁵,2-Nal³¹)hGLP-1(7-36)NH₂; | (SEQ ID NO.741) |
| (Ser⁸,Aib³⁵,2-Nal^{28,31})hGLP-1(7-36)NH₂; | (SEQ ID NO.742) |
| (Ser⁸,Aib³⁵,Arg^{26,34},2-Nal³¹)hGLP-1(7-36)NH₂; | (SEQ ID NO.743) |
| (Ser⁸,Aib³⁵,Arg^{26,34},Phe³¹)hGLP-1(7-36)NH₂; | (SEQ ID NO.744) |
| (Ser⁸,Aib³⁵,2-Nal^{19,31})hGLP-1(7-36)NH₂; | (SEQ ID NO.745) |
| (Ser⁸,Aib³⁵,2-Nal^{12,31})hGLP-1(7-36)NH₂; | (SEQ ID NO.746) |
| (Ser⁸,Aib³⁵,Lys³⁶(N^{ε}-decanoyl))hGLP-1(7-36) NH₂; | (SEQ ID NO.483) |
| (Ser⁸,Aib³⁵,Arg³⁴,Lys²⁶(N^{ε}-decanoyl))hGLP-1(7-36)NH₂; | (SEQ ID NO.748) |
| (Ser⁸,Aib³⁵,Arg^{26,34},Lys³⁶(N^{ε}-dodecanoyl))hGLP-1(7-36)NH₂; | (SEQ ID NO.749) |
| (Ser⁸,β-Ala³⁵,Ser³⁷(O-decanoyl))hGLP1(7-37)-NH₂; | (SEQ ID NO.539) |
| (Ser⁸,Aib²⁷,β-Ala^{35,37},Arg³⁸,Lys³⁹(N^{ε}-octanoyl))hGLP-1(7-39)NH₂; | (SEQ ID NO.751) |
| (Ser⁸,Arg^{26,34},β-Ala³⁵,Lys³⁷(N^{ε}-octanoyl))hGLP-1(7-37)NH₂; | (SEQ ID NO.752) |
| (Ser⁸,Arg^{26,34},β-Ala³⁵,Lys³⁷(N^{ε}-decanoyl))hGLP-1(7-37)NH₂; | (SEQ ID.NO.753) |
| or | |
| (Ser⁸,Arg^{26,34},β-Ala³⁵,Lys³⁷(N^{ε}-tetradecanoyl))hGLP-1(7-37)NH₂; | (SEQ ID NO.754) |

or a pharmaceutically acceptable salt thereof.

15. A compound according to claim 1, wherein said compound is:

| | |
|---|---|
| Ser⁸,Aib³⁵)hGLP-1(7-36)NH₂; | (SEQ ID NO.767) |
| (Abu⁸,β-Ala³⁵)hGLP-1(7-36)NH₂; | (SEQ ID NO.768) |
| (Val⁸,β-Ala³⁵)hGLP-1(7-36)NH₂; | (SEQ ID NO.769) |
| (β-Ala³⁵)hGLP-1(7-36)NH₂; | (SEQ ID NO.770) |
| (Abu⁸,Aib³⁵)hGLP-1(7-36)NH₂; | (SEQ ID NO.771) |
| (Val⁸,Aib³⁵)hGLP-1(7-36)NH₂; | (SEQ ID NO.772) |
| or | |
| (β-Ala⁸,Aib³⁵)hGLP-1(7-36)NH₂; | (SEQ ID NO.773) |

or a pharmaceutically acceptable salt thereof.

16. A compound according to claim 15, wherein said compound is:

| | |
|---|---|
| Ser⁸, Aib³⁵)hGLP-1(7-36)NH₂; | (SEQ ID NO.767) |
| (Abu⁸,β-Ala³⁵)hGLP-1(7-36)NH₂; | (SEQ ID NO.768) |
| or | |
| (Abu⁸,Aib³⁵)hGLP-1(7-36)NH₂; | (SEQ ID NO.771) |

or a pharmaceutically acceptable salt thereof.

17. A compound according to claim 16, wherein said compound is:

| | |
|---|---|
| Ser⁸,Aib³⁵)hGLP-1(7-36)NH₂; | (SEQ ID NO.767) |

or a pharmaceutically acceptable salt thereof.

18. A pharmaceutical composition comprising an effective amount of a compound according to claim 1 or a pharmaceutically acceptable salt thereof and a pharmaceutically acceptable carrier or diluent.

19. A method of eliciting an agonist effect from a GLP-1 receptor in a subject in need thereof which comprises administering to said subject an effective amount of a compound according to claim 1 or a pharmaceutically acceptable salt thereof.

20. A method of treating a disease selected from the group consisting of Type I diabetes, Type II diabetes, obesity, glucagonomas, secretory disorders of the airway, metabolic disorder, arthritis, osteoporosis, central nervous system disease, restenosis and neurodegenerative disease, in a subject in need thereof which comprises administering to said subject an effective amount of a compound according to claim 1 or a pharmaceutically acceptable salt thereof.

21. A method according to claim 20 wherein said disease is Type I diabetes or Type II diabetes.
